(19) United States
(12) Patent Application Publication
(10) Pub. No.: US 2022/0089775 A1
(43) Pub. Date: Mar. 24, 2022

(54) MULTIPLY AUXOTROPHIC CELL LINE FOR THE PRODUCTION OF RECOMBINANT PROTEINS AND METHODS THEREOF

(71) Applicants: THE TRUSTEES OF COLUMBIA UNIVERSITY IN THE CITY OF NEW YORK, New York, NY (US); MERCK SHARP & DOHME CORP, Rahway, NJ (US)

(72) Inventors: Lawrence Chasin, Leonia, NJ (US); Qinghao Zhang, Fort Lee, NJ (US); Zhimei Du, Kenilworth, NJ (US)

(21) Appl. No.: 17/536,716

(22) Filed: Nov. 29, 2021

Related U.S. Application Data
(63) Continuation-in-part of application No. PCT/US20/35420, filed on May 29, 2020.
(60) Provisional application No. 62/855,462, filed on May 31, 2019.

(51) Int. Cl. C07K 16/32; C12N 15/87; C12N 15/64

(52) U.S. Cl. CPC C07K 16/32; C12N 15/87; C07K 2319/30; C07K 2317/14; C12N 15/64

(57) ABSTRACT

The present invention provides, inter alia, a multiply auxotrophic cell line that is deficient in genes encoding enzymes that catalyze steps in the de novo synthesis of the pyrimidine and purine pathways, such as, e.g., uridine monophosphate synthetase (UMPS) and 5-aminoimidazole-4-carboxamide ribonucleotide formyltransferase/IMP cyclohydrolase (ATIC), respectively, for the production of recombinant proteins such as recombinant monoclonal and bispecific antibodies. Methods for preparing the multiply auxotrophic, in particular the doubly auxotrophic and octa-auxotrophic cell lines disclosed herein, methods for selecting a cell expressing a protein of interest, methods for producing a protein of interest, methods for optimizing the activity of a protein of interest, and kits for selecting a cell expressing a protein of interest, are also provided. In addition, recombinant proteins such as antibodies, including monoclonal and bispecific antibodies, made by the methods of the present disclosure are also provided.

### CROSS REFERENCE TO RELATED APPLICATIONS

The present application is a continuation-in-part of PCT international application no. PCT/US2020/035420, filed on May 29, 2020, which claims benefit of U.S. Provisional Patent Application Ser. No. 62/855,462, filed on May 31, 2019, which application are incorporated by reference herein in their entireties.

### FIELD

The present invention provides, inter alia, a multiply auxotrophic cell line that is deficient in genes encoding enzymes that catalyze steps in the de novo synthesis of the pyrimidine and purine pathways, such as, e.g., uridine monophosphate synthetase (UMPS) and 5-aminoimidazole-4-carboxamide ribonucleotide formyltransferase/IMP cyclohydrolase (ATIC), respectively, for the production of recombinant proteins. Methods and kits for preparing and using such cell line are also provided. Antibodies, including monoclonal and bispecific antibodies, made by the methods disclosed herein are also provided.

### INCORPORATION BY REFERENCE OF SEQUENCE LISTING

This application contains references to amino acids and/or nucleic acid sequences that have been filed concurrently herewith as sequence listing text file “CU19274-seq-update.txt”, file size of 28 KB, created on Nov. 26, 2021. The aforementioned sequence listing is hereby incorporated by reference in its entirety pursuant to 37 C.F.R. § 1.52(e)(5).

### BACKGROUND

In the past decades, biopharmaceuticals have become more and more pivotal in the development of novel and innovative therapeutics in academic and industrial environments. Currently, mammalian cells are the major means used to produce the high quality and quantity of biopharmaceuticals, most of them being monoclonal antibodies (mAbs), to meet the increasing demands in clinical uses (Fischer, Handrick, & Otte, 2015). As the workhorse of mammalian hosts, CHO (Chinese hamster ovary) cells have been employed to produce 70% of therapeutic recombinant proteins due to their ease of transfection, production of glycan structures similar to those of human secreted proteins, easy adaptation to suspension medium, refractoriness to human viruses, and growth to high densities (Fischer et al., 2015; Lalonde & Durocher, 2017; Rita Costa, Elisa Rodrigues, Henriques, Azeredo, & Oliveira, 2010). In 2015 and 2016, more than half of newly approved biotherapeutics were produced in CHO cells (Lalonde & Durocher, 2017).

Tremendous efforts have been made to improve the productivity of recombinant proteins in CHO cells in time-saving and cost-efficient ways. With the help of optimization of culture medium, engineering of transgenes and vectors, and targeted engineering of host cells yields of recombinant proteins using CHO cells has reached 10 g/L (Kuo et al., 2018). Most current therapeutic recombinant proteins are mAbs, which consist of both light and heavy chain subunits and these polypeptides are encoded by 2 separate genes. In this case it would be advantageous to have a way to co-select both genes without using synthetic chemicals and/or antibiotics. A multiply auxotrophic cell line would allow selection of multiple independently controlled vectors using a medium that is simply lacking the multiple required nutrients, e.g., without the use of any supplements during fed-batch production.

### SUMMARY

CHO cells are the most widely used mammalian hosts for recombinant protein production due to their hardiness, ease of transfection, and production of glycan structures similar to those observed in natural human mAbs. To enhance the usefulness of CHO-K1 cells, a new selection system was developed based on double auxotrophy. CRISPR-Cas9 was used to knockout the genes that encode bifunctional enzymes catalyzing the last two steps in the de novo synthesis of pyrimidines and purines (UMPS and ATIC, respectively). Survival of these doubly auxotrophic cells depends either on the provision of sources of purines and pyrimidines or on the transfection and integration of minigenes encoding these two enzymes. One such double auxotroph (UA10) was successfully used to select for stable transfectants carrying 1) the recombinant TNFα receptor fusion protein Enbrel and 2) the heavy and light chains of the anti-Her2 monoclonal antibody Herceptin. Transfectant clones produced these recombinant proteins in a stable manner and in substantial amounts. The availability of this double auxotroph provides a rapid and efficient selection method for the serial or simultaneous transfer of genes for multiple polypeptides of interest into CHO cells using readily available purine- and pyrimidine-free commercial media.

Accordingly, the present disclosure provides a doubly auxotrophic CHO cell line deficient in genes encoding 5-aminoimidazole-4-carboxamide ribonucleotide formyltransferase/IMP cyclohydrolase (ATIC) and uridine monophosphate synthetase (UMPS) disrupting the purine and pyrimidine de novo synthesis pathways, respectively. Employment of this cell line in the production of a model antibody, Herceptin, showed that high productivity clones (10 out of 12 randomly picked clones) could be obtained within two months and the clones sustained high productivity for at least 3 months of continuous culture (22 passages) in the selection medium, a commercially available formulation that contains no toxic chemicals.

One embodiment of the present disclosure is a multiply auxotrophic cell line that is deficient in (i) at least one gene encoding an enzyme in the de novo pathway for pyrimidine nucleotide synthesis and (ii) at least one gene encoding an enzyme in the de novo pathway for purine nucleotide synthesis.

Another embodiment of the present disclosure is a doubly auxotrophic cell line that is deficient in the gene encoding uridine monophosphate synthetase (UMPS) and the gene encoding 5-aminoimidazole-4-carboxamide ribonucleotide formyltransferase/IMP cyclohydrolase (ATIC).

Another embodiment of the present disclosure is a method for preparing a doubly auxotrophic cell line disclosed herein, comprising the steps of: (a) knocking out a UMPS gene from the genome of the cell line; (b) growing cells from step (a) in a medium containing 5-fluoroorotic acid (5-FOA) and uridine; (c) selecting cells that survive in step (b) and further knocking out an ATIC gene from the genome of the surviving cells; (d) growing clones of cells from step (c) in duplicate in both (i) a medium containing uridine but no hypoxanthine and (ii) a complete medium; and (e) if the cells do not survive in (d-i), collecting their counterparts in (d-ii) as the doubly auxotrophic cells.

An additional embodiment of the present disclosure is a method for selecting a cell expressing a protein of interest, comprising the steps of: (a) obtaining a doubly auxotrophic cell line disclosed herein; (b) constructing a first vector carrying a UMPS open reading frame (ORF) and a first coding sequence of the protein of interest; (c) constructing a second vector carrying an ATIC open reading frame (ORF) and a second coding sequence of the protein of interest; (d) transfecting the doubly auxotrophic cells with the first and second vectors; (e) incubating the transfected cells from step (d) in a medium that lacks sources of purines and pyrimidines; and (f) selecting a cell that survives in step (e) as the cell expressing the protein of interest.

Another embodiment of the present disclosure is a method for producing a protein of interest, comprising: (a) obtaining a doubly auxotrophic cell line disclosed herein; (b) constructing a first vector carrying a UMPS open reading frame (ORF) and a first coding sequence of the protein of interest; (c) constructing a second vector carrying an ATIC open reading frame (ORF) and a second coding sequence of the protein of interest; (d) transfecting the doubly auxotrophic cells with the first and second vectors; (e) incubating the transfected cells from step (d) in a medium that lacks sources of purines and pyrimidines; (f) selecting a cell that survives in step (e) as the cell expressing the protein of interest; and (g) producing the protein of interest by culturing the cell selected in step (f).

A further embodiment of the present disclosure is a kit for selecting a cell expressing a protein of interest, comprising: i) a doubly auxotrophic cell line disclosed herein; ii) a first vector carrying a UMPS open reading frame (ORF) and a first coding sequence of the protein of interest; iii) a second vector carrying an ATIC open reading frame (ORF) and a second coding sequence of the protein of interest; iv) a medium that lacks sources of purines and pyrimidines; and v) instructions of use.

Another embodiment of the present disclosure is a recombinant protein as disclosed herein made by the processes disclosed herein.

Yet another embodiment of the present disclosure is a monoclonal antibody made by the processes disclosed herein.

Still another embodiment of the present disclosure is a bispecific antibody made by the processes disclosed herein.

Another embodiment of the present disclosure is a multiply auxotrophic cell line deficient in five enzymatic activities and that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), and the gene encoding 5-aminoimidazole-4-carboxamide ribonucleotide formyltransferase/IMP cyclohydrolase (ATIC).

Another embodiment of the present disclosure is a multiply auxotrophic cell line deficient in seven enzymatic activities and that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), the gene encoding 5-aminoimidazole-4-carboxamide ribonucleotide formyltransferase/IMP cyclohydrolase (ATIC), the gene encoding phosphoribosylaminoimidazole carboxylase (PAICS), and the gene encoding guanosine monophosphate synthetase (GMPS).

Another embodiment of the present disclosure is a multiply auxotrophic cell line that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), the gene encoding 5-aminoimidazole-4-carboxamide ribonucleotide formyltransferase/IMP cyclohydrolase (ATIC), the gene encoding phosphoribosylaminoimidazole carboxylase (PAICS), the genes encoding inosine-5′-monophosphate dehydrogenase 1 and 2 (IMPDH1/2), and the gene encoding guanosine monophosphate synthetase (GMPS).

Another embodiment of the present disclosure is a multiply auxotrophic cell line deficient in ten enzymatic activities and that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), the gene encoding 5-aminoimidazole-4-carboxamide ribonucleotide formyltransferase/IMP cyclohydrolase (ATIC), the gene encoding phosphoribosylaminoimidazole carboxylase (PAICS), the gene encoding adenylosuccinate lyase (ADSL), the genes encoding inosine-5′-monophosphate dehydrogenase 1 and 2 (IMPDH1/2), the gene encoding guanosine monophosphate synthetase (GMPS), and the genes encoding adenylosuccinate synthase and adenylosuccinate synthase like 1 (ADSS/ADSSL1).

Still another embodiment of the present disclosure is a method for modulating recombinant monoclonal antibody production, comprising: (a) obtaining a multiply auxotrophic cell line disclosed herein; (b) constructing a vector carrying i) the open reading frame (ORF) of a required enzyme; and ii) a coding sequence of the heavy chain of the recombinant monoclonal antibody; or a coding sequence of the light chain of the recombinant monoclonal antibody; (c) constructing another vector according to step (b) with a different required enzyme; (d) repeating step (c) until each of the required enzymes is carried by one vector, and at least one of the vectors carries the coding sequence of the heavy chain or light chain of the recombinant monoclonal antibody or each of the vectors carries the coding sequence of the heavy chain or light chain of the recombinant monoclonal antibody; (e) transfecting the multiply auxotrophic cells with all the vectors; (f) incubating the transfected cells from step (e) in a medium that lacks sources of purines and pyrimidines; (g) selecting a cell that survives in step (f) as the cell expressing the recombinant monoclonal antibody; and (h) producing the recombinant monoclonal antibody by culturing the cell selected in step (g).

Another embodiment of the present disclosure is a method for producing a multi-subunit protein of interest, comprising: (a) obtaining a multiply auxotrophic cell line disclosed herein; (b) constructing a vector carrying i) the open reading frame (ORF) of a required enzyme; and ii) the coding sequence of a subunit of the protein of interest; (c) constructing another vector according to step (b) with a different required enzyme and the coding sequence of a different subunit of the protein of interest; (d) repeating step (c) until each subunit of the protein of interest is carried by at least one vector carrying a different required enzyme; (e) transfecting the multiply auxotrophic cells with all the vectors; (f) incubating the transfected cells from step (e) in a medium that lacks sources of purines and pyrimidines; (g) selecting a cell that survives in step (f) as the cell expressing the multi-subunit protein of interest; and (h) producing the multi-subunit protein of interest by culturing the cell selected in step (g).

Another embodiment of the present disclosure is a method for optimizing the activity of a protein of interest, comprising: (a) obtaining a multiply auxotrophic cell line disclosed herein that expresses the protein of interest; (b) constructing a vector carrying i) the open reading frame (ORF) of a required enzyme; and ii) the coding sequence of an enzyme that can modulate the activity of the protein of interest; (c) constructing another vector according to step (b) with a different required enzyme and the coding sequence of a different enzyme that can modulate the activity of the protein of interest; (d) repeating step (c) as necessary until each enzyme that can modulate the activity of the protein of interest is carried by at least one vector carrying a different required enzyme; (e) transfecting the multiply auxotrophic cells with all the vectors; (f) incubating the transfected cells from step (e) in a medium that lacks sources of purines and pyrimidines; (g) selecting a cell that survives in step (f) as the cell expressing the protein of interest having desired activity; and (h) producing the protein of interest having desired activity by culturing the cell selected in step (g).

The present disclosure also extended the auxotrophies of the cell lines from 2 to 8 by knocking out the genes for additional enzymes in the purine and pyrimidine pathways. The new cell line, CHO-8A, is deficient in Dhodh, Umps, Ctps1, Ctps2, Tyms, Paics, Atic, Impdh1, Impdh2 and Gmps in these pathways as shown by coding changes in their DNA sequences and their inability to grow without provision of appropriate nutrients. Stepwise expression of the 8 rescued enzymes in various combinations (DOHDH, UMPS, CTPS1, TYMS, PAICS, ATIC, IMPDH2 and GMPS) demonstrated no compensatory activities among them and the rescued enzymes conferred the CHO-8A cells with the ability to survive in the selective medium. CHO-8A cells manifested favorable properties in the production of a model antibody, trastuzumab (Herceptin), which could be applied to other recombinant proteins in several ways: 1) rapid isolation of cell clones permanently expressing recombinant protein with up to 8 subunits (multiple light and heavy chains in the current case) within 2 months; 2) no antibiotics or drugs are needed for selection 3) high productivity (up to 83 pcd) in a substantial proportion of the isolated cell clone; and 4) flexibility in allocation of transgenes of interest (8 transgenes in current case; 1 through 8 transgenes could be utilized readily by adjustment of nutrients supplemented in the selective medium). In conclusion, CHO-8A cells provide a promising platform for flexible and rapid isolation of permanent CHO cell clones expressing high levels of recombinant proteins.

Accordingly, a further embodiment of the present disclosure is an octa-auxotrophic cell line that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), the gene encoding 5-aminoimidazole-4-carboxamide ribonucleotide formyltransferase/IMP cyclohydrolase (ATIC), the gene encoding phosphoribosylaminoimidazole carboxylase (PAICS), the genes encoding inosine-5′-monophosphate dehydrogenase 1 and 2 (IMPDH1/2), and the gene encoding guanosine monophosphate synthetase (GMPS).

Another embodiment of the present disclosure is a method for preparing an octa-auxotrophic cell line disclosed herein, comprising the steps of: (a) obtaining a doubly auxotrophic cell line disclosed herein; (b) knocking out genes DHODH, TYMS, CTPS1 and CTPS2 from the genome of the doubly auxotrophic cell line obtained in step (a); (c) growing colonies of cells from step (b) in duplicate in both (i) a selective medium containing none of thymidine, cytidine, uridine and hypoxanthine and (ii) a complete medium; (d) if the cells do not survive in (c-i), collecting their counterparts in (c-ii) and further confirming the knock-out of DHODH, TYMS, CTPS1 and CTPS2 by DNA-sequencing; (e) selecting cells with confirmed knock-out of DHODH, TYMS, CTPS1 and CTPS2 in step (d) and further knocking out genes GMPS and PAICS from the genome of the selected cells; (f) growing clones of cells from step (e) in duplicate in both (i) a selective medium containing none of thymidine, cytidine, guanine, uridine and hypoxanthine and (ii) a complete medium; (g) if the cells do not survive in (f-i), collecting their counterparts in (f-ii) and further confirming the knock-out of GMPS and PAICS by DNA-sequencing; (h) selecting cells with confirmed knock-out of GMPS and PAICS in step (g) and further knocking out genes IMPDH1 and IMPDH2 from the genome of the selected cells; (i) growing clones of cells from step (h) in a complete medium and confirming the knock-out of IMPDH1 and IMPDH2 by DNA-sequencing; and (j) selecting the cells confirmed in step (i) as the octa-auxotrophic cells.

Another embodiment of the present disclosure is a method for modulating recombinant monoclonal antibody production, comprising: (a) constructing a vector carrying i) the open reading frame (ORF) of a required enzyme for an octa-auxotrophic cell line; and ii) a coding sequence of the heavy chain of the recombinant monoclonal antibody; or a coding sequence of the light chain of the recombinant monoclonal antibody; (b) constructing another vector according to step (a) with a different required enzyme; (c) repeating step (b) until each of the required enzymes is carried by one vector, and at least one of the vectors s carries the coding sequence of the heavy chain or light chain of the recombinant monoclonal antibody; (d) transfecting the octa-auxotrophic cell line with all the vectors; (e) incubating the transfected cells from step (d) in a medium that lacks sources of purines and pyrimidines; (f) selecting a cell that survives in step (e) as the cell expressing the recombinant monoclonal antibody; and (g) producing the recombinant monoclonal antibody by culturing the cell selected in step (f).

Another embodiment of the present disclosure is a method for modulating recombinant monoclonal antibody production, comprising: (a) constructing a vector carrying i) the open reading frame (ORF) of a required enzyme for an octa-auxotrophic cell line; and ii) a coding sequence of the heavy chain of the recombinant monoclonal antibody; and a coding sequence of the light chain of the recombinant monoclonal antibody; (b) constructing another vector according to step (a) with a different required enzyme; (c) repeating step (b) until each of the required enzymes is carried by one vector; (d) transfecting the octa-auxotrophic cell line with all the vectors; (e) incubating the transfected cells from step (d) in a medium that lacks sources of purines and pyrimidines; (f) selecting a cell that survives in step (e) as the cell expressing the recombinant monoclonal antibody; and (g) producing the recombinant monoclonal antibody by culturing the cell selected in step (f).

Another embodiment of the present disclosure is a method for protein production, comprising: (a) constructing a vector carrying (i) the open reading frame (ORF) of a required enzyme for an octa-auxotrophic cell line; and (ii) a coding sequence of one or more proteins or protein subunits of interest; (b) constructing another vector according to step (a) with a different required enzyme; (c) repeating step (b) until each of the required enzymes is carried by at least one vector, and at least one of the vectors carries the coding sequence of the one or more proteins or protein subunits of interest or each of the vectors carries the coding sequence of the one or more proteins or protein subunits of interest; (d) transfecting the octa-auxotrophic cell line with the constructed vectors; (e) incubating the transfected cells from step (d) in a medium that lacks sources of purines and pyrimidines; (f) selecting a cell that survives in step (e) as the cell expressing the one or more protein or protein subunits of interest; and (g) producing the one or more protein or protein subunits by culturing the cell selected in step (f).

Another embodiment of the present disclosure is an octa-auxotrphic cell line made by any process disclosed herein. In some embodiments, the cell line is selected from the group consisting of HEK293, HEK293T, BHK21, CHO, CHO/dhfr−, CHO-K1, NS0, Sp2/0-Ag14, and Sp2/0-Ag14-TurboDoma. In some embodiments, the cell line is a CHO cell line. In some embodiments, the cell line is a CHO-K1 cell line.

In some embodiments, a protein of interest produced by the methods disclosed herein is effective as an antigen for vaccine production. In some embodiments, the protein of interest is selected from the group consisting of the spike protein subunits and the NP protein of SARS Coy 2 virus and the gp120 envelope protein from the HIV virus, and combinations thereof. In some embodiments, one or more proteins or protein subunits of interest produced by the methods disclosed herein are effective as an antigen for vaccine production. In some embodiments, the one or more proteins or protein subunits of interest are selected from the group consisting of the spike protein subunits and the NP protein of SARS Coy 2 virus and the gp120 envelope protein from the HIV virus, and combinations thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B show the de novo biosynthetic pathways of purines and pyrimidines. The exemplary enzymes whose genes are targeted in this example, Umps and Atic, are shown highlighted and in bold, The nutrients that can satisfy the auxotrophic requirements created in this example are shown boxed.

FIG. 1A shows the de novo pyrimidine synthesis. This pathway initiates from carbon dioxide, glutamine and ATP to produce carbamoyl phosphate (CAP). The subsequent intermediate products include carbamoyl aspartic acid (CAA), dihydroorotic acid (DHOA), orotic acid (OA), and oritidine-5′-monophosphate (OMP). One end-product is uridine monophosphate (UMP) formed by uridine monophosphate synthase (Umps), a bifunctional enzyme with orotate phosphoribosyltransferase and orotidine-5′-phosphate decarboxylase activities. UMP is further converted to thymidine monophosphate (TMP) and cytidine triphosphate (CTP), both of which are indispensable for nucleic acid metabolism. An analog of OA, 5-fluoroorotic acid (5-FOA, structure shown in shade) is incorporated into the synthetic pathway when present in the medium. This process converts OA to the toxic analog 5-fluoro-UMP.

FIG. 1B shows the de novo purine synthesis. The synthesis of 5-phosphoribosylamine (PRA) from 5-phosphoribosylpyrophosphate (PRPP, chemical structure shown) is catalyzed by amidophosphoribosyl transferase. PRA is further metabolized to produce a series of intermediates including glycinamide ribotide (GAR), formylglycinamide ribotide (FGAR), formylglycinamidine ribotide (FGAM), aminoimidazole ribotide (AIR), carboxyaminoimidazole ribotide (CAIR), 5-aminoimidazole-4-(N-succinylocarboxamide) ribotide (SAICAR) and 5-aminoimidazole-4-carboxamide ribotide (AICAR). AICAR can be converted to 5-formaminoimidazole-4-carboxamide ribotide (FAICAR) and inosine monophosphate (IMP, chemical structure is shown) by a bifunctional enzyme Atic, having 5-aminoimidazole-4-carboxamide ribonucleotide formyltransferase/IMP cyclohydrolase activities. IMP is the precursor of both adenosine monophosphate (AMP) and guanosine monophosphate (GMP).

FIGS. 2A-2B show the analysis of PCR products from mutated UMPS and ATIC genes in UMPS and UMPS/ATIC knockouts. DNA from mutant cell clones and parental CHO-K1 cells was amplified by PCR with primers targeting the region surrounding gRNA targets. The PCR products were separated on a 2.5% agarose gel and then subjected to Sanger sequencing.

FIG. 2A shows that two surviving clones from the 5-FOA selection (U1 and U3) were chosen for checking UMPS DNA sequences. PCR product mobilities are similar, but sequencing showed that U1 had an insertion of a single A and U3 had an 8 base deletion.

FIG. 2B shows that twelve doubly auxotrophic single clones that could not survive in −H+U medium (−: absence; +: presence; H: hypoxanthine; U: uridine) were chosen for ATIC DNA sequence examination. Seven mutants produced PCR product similar in size to that of parental U3 cells. Three showed 2 or more bands, suggesting complex mutations, diploidy at the ATIC locus or non-clonality. UA9 and UA10 showed single PCR products of higher or lower size, respectively; sequencing revealed a large insertion in UA9 and a large deletion in UA10, as expected. UA7 contained the insertion of a single T at the target site. The expected cutting sites for Cas9 are indicated by arrowheads.

FIG. 3 shows that clones UA7 and UA10 required a source of both purines and pyrimidines for growth. Cells (50,000) were seeded in −H−U medium with or without supplements of 100 μM hypoxanthine (+H−U) and/or 100 μM uridine (−H+U). After 7 days incubation, the cells were stained with crystal violet. CHO-K1 were incubated in complete, −H−U or −H−U media supplemented with H and U (+H+U) in parallel.

FIGS. 4A-4C show the rescued expression of UMPS and ATIC along with Enbrel or Herceptin in doubly auxotrophic UA10 cells.

FIG. 4A shows the schematics of Enbrel vectors used for the rescued expression in doubly auxotrophic UA10 cell. VU and VA vectors were constructed by replacement of the IRES-driven NeoR ORF in the pIRESneo3 vector with UMPS or ATIC ORFs, respectively. To express Enbrel its ORF was cloned into the NsiI site downstream of the IVS in VU and VA to form VUE and VAE, respectively.

FIG. 4B shows that single or combinations of vectors were transfected or co-transfected into UA10 cells, as indicated. Two days after transfection, the cells were seeded (5×10⁴ or 3×10⁵ per 100 mm dish) in −H−U medium with or without supplements of either 100 μM hypoxanthine (+H) or 100 μM uridine (+U). After 7 additional days of culture, the cells were stained with crystal violet. UA10 cells were seen to require both the UMPS and the ATIC genes in order to grow in a medium lacking both purines and pyrimidines.

FIG. 4C shows the schematics of vectors constructed as in FIG. 4A but combining UMPS and Herceptin light chain (UL), ATIC and Herceptin heavy chain (AH), UMPS and Herceptin heavy chain (UH), or ATIC and Herceptin light chain (AL).

FIG. 5 shows the stability of Herceptin expression in UA10 cells. The UA10 cells (clone G of UH+AL in Table 3) were continuously cultured in −H−U medium. After the indicated number of weeks cells were collected and used for determination of Herceptin expression by ELISA. Each point represents the percentage of the mean productivity relative to the control (day 0). The SEMs of triplicate well secretion measurements using 10⁶ cells each are shown.

FIG. 6 shows the pyrimidine and purine de novo syntheses. De novo pyrimidine synthesis: this pathway initiates from carbon dioxide, glutamine and ATP to produce carbamoyl phosphate (CAP). The subsequent intermediate products include carbamoyl aspartic acid (CAA), dihydroorotic acid (DHOA), orotic acid (OA), and oritidine-5′-monophosphate (OMP), among which DHODH catalyzes the reaction from DHOA to OA. OA is a substrate of uridine monophosphate synthase (Umps), a bifunctional enzyme with orotate phosphoribosyltransferase and orotidine-5′-phosphate decarboxylase activities, to finally produce UMP. UMP is further converted to thymidine triphosphate (TTP) and to cytidine triphosphate (CTP); CTPS1 or CTPS2 is responsible for this last step, conversion of UTP to CTP; TYMS is responsible for conversion of dUMP to dTMP. De novo purine synthesis: the synthesis of 5-phosphoribosylamine (PRA) from 5-phosphoribosylpyrophosphate (PRPP) and glutamine is catalyzed by amidophosphoribosyl transferase. PRA is further metabolized to produce a series of intermediates including glycinamide ribotide (GAR), formylglycinamide ribotide (FGAR), formylglycinamidine ribotide (FGAM), aminoimidazole ribotide (AIR), carboxyaminoimidazole ribotide (CAIR), 5-aminoimidazole-4-(N-succinylocarboxamide) ribotide (SAICAR) and 5-aminoimidazole-4-carboxamide ribotide (AICAR). AICAR can be converted to 5-formaminoimidazole-4-carboxamide ribotide (FAICAR) and then to inosine monophosphate (IMP) by the bifunctional enzyme Atic, having 5-aminoimidazole-4-carboxamide ribonucleotide formyltransferase/IMP cyclohydrolase activities. IMP is the precursor of both adenosine monophosphate (AMP) and guanosine monophosphate (GMP); production of the latter is catalyzed by IMPDH 1 or 2 and then GMPS. The enzymes, whose genes are targeted here, are shown in bold. Umps and Atic that were knocked out to create UA10 cells as described in Example 1 (see also Zhang et al. 2020) are underlined. The nutrients that can satisfy the auxotrophic requirements created here (see below) are shown boxed.

FIGS. 7A-7C show the knockout of enzymes in pyrimidine and purine synthesis pathway of UA10 cells.

FIG. 7A shows the nutrients requiring phenotype testing for CHO-5A with Umps, Atic, Dhodh, Ctps1, Ctps2 and Tyms knocked out and CHO-7A cells with Umps, Atic, Dhodh, Ctps1, Ctps2, Tyms, Paics and Gmps knocked out. The cells transfected with respective Crispr-Cas9 vector were then challenged in the selective medium supplemented with uridine (U), hypoxanthine (H), thymidine (T), cytidine (C) and guanine (G) in various combinations to test the deficiencies of the enzymes.

FIG. 7B shows that the genotypes corresponding to each enzyme knocked out were measured by Sanger sequencing or deep sequencing. The underlined sequences indicate the gRNA targets. The insertion nucleotide(s) are shown in italic and in bold. The deleted nucleotides are replaced by dash symbol (-) or number (93 nt).

FIG. 7C shows the cell growth rates of CHO-8A in the complete medium supplemented with UCTAG. The cells were seeded in six-well plates at 5,000 cells/well. The cell number was counted daily by hemocytometer from Day 2 to Day 13 of culture. The number of viable (trypan blue excluding) cells per well included both adhered cells and viable cells shed into the medium at high densities. The doubling time in the exponential growth phase (days 2-8) was calculated based on the equation: doubling time=ln(2)/k where k is the slope of the best fit line (using Excel) for points from Day 2 to Day 8 inclusive. The doubling time was 16.6 hours.

FIG. 8 shows the rescue expression of the enzymes knocked out in CHO-8A cells. Single or combinations of up to 8 vectors carrying expression ORFs for each the 8 enzymes were transfected or co-transfected (+), or not (−) into CHO-8A cells, with vectors named as indicated in the table at the lower right. Two days after transfection, the cells were seeded (5×10⁴ per 100 mm dish) in the selective medium supplemented with various combinations of uridine (U); thymidine (T); cytidine (C); hypoxanthine (H); adenine (A); guanine (G). After 10 additional days of culture, the cells were stained with crystal violet. Based on the supplemented nutrients CHO-8A cells were divided into 9 groups, where ∘ depicts the absence of the nutrients and ● depicts the presence of the nutrients. Note that if A and G are provided, H need not be. The schematic diagram in the left corner shows the enzymes required in the indicated groups.

FIGS. 9A-9B show a representative application of CHO-8A cells in production of trastuzumab (Herceptin).

FIG. 9A shows schematics of vectors used for the rescued expression of DHODH, UMPS, CTPS1, TYMS, PAICS, ATIC, IMPDH2 or GMPS plus trastuzumab. Two sets (1 and 2) of vectors were constructed with each set having 8 rescue vectors. Set 1 is comprised of bicistronic vectors where the ORF of one light chain or one heavy chain of trastuzumab was placed before a strong (wt) internal ribosome entry site (IRES) followed by the ORF of one of the rescue enzymes driven by a weak IRES. The arrangements in Set 2 (tricistronic vectors) were the same as those in Set 1 except that ORFs of the light chain and the heavy chain were placed together in one vector and split by an additional strong IRES (IRES_wt). All of the vectors had inverted terminal repeat (ITR) sequences placed before the CMV promoter and after the SV40 pA sequence, as the sites recognized by Sleeping Beauty transposase (SB100X). Constructions of the vectors are detailed in Example 2, Materials and Methods section.

FIG. 9B shows the productivity of trastuzumab in CHO-8A cells transfected with Set 1 or Set 2 vectors with or without the Sleeping Beauty 100X (SB100X) transposase vector. Set 1 or Set 2 of vectors together with or without SB100X was transfected into CHO-8A cells. Two days later the cells were subjected to selection for 10 days in the selective medium without any supplemented nutrients. The cell clones were then isolated and incubated for measurement by ELISA of secreted trastuzumab in the medium. Each dot represents the productivity of each clone isolated (pg/cell/day, pcd) and the mean values and SD bars for each group are also shown.

FIG. 10 shows the sequence information of the Block sequence and IRES_wt as described in Example 2.

FIG. 11 shows rescued expression of the enzymes knocked out in CHO8A cells. Single or combinations of 8 vectors, each carrying an ORFs of one of the 8 rescue enzymes (RE), were transfected into CHO8A cells. The transfectants were then selected in purine/pyrimidine-free medium supplemented with various combinations of uridine (U), thymidine (T), cytidine (C), hypoxanthine (H), adenine (A), and guanine (G), where an open circle denotes the absence of a nutrient and a filled circle denotes its presence. Transfections were divided into 8 groups (a-h) based on the nutrients provided. Note that if A is provided, H need not be and vice versa. The diagram in the left lower corner shows the enzymatic steps knocked out with a number for each step. The numbers under each staining picture indicates the enzymatic steps circumvented or rescued: numbers with grey shading denote those steps rescued by provision of the nutrients indicated by the filled circles at the top; numbers with dark shading denote those steps/enzymes rescued by the vectors used for transfection. The observed growth of cells is indicated in the upper left corner of each staining picture: −, no growth; −/+, large dispersed cells; +/−, loosely packed cells; +/+ densely packed cells.

FIGS. 12A-12E show production of mAbs trastuzumab and 9-24 in CHO8A cells.

FIG. 12A shows a schematic of the vectors used for expression of light (LC) or heavy chain (HC) of trastuzumab and 9-24. The vectors are bicistronic: the ORF of a LC or HC driven by a CMV promoter is followed by an internal ribosome entry site (IRES)-driven ORF of one of the rescue enzymes. All of the vectors had inverted SB100X terminal repeat (ITR) sequences for integration.

FIG. 12B shows specific productivity of trastuzumab and 9-24 in CHO8A cell clones selected for growth in purine/pyrimidine-free medium after simultaneous transfection with the 8 rescue vectors along with (TrasSB, 9-24SB) or without (Tras) an SB100X transposase expression vector. Each dot represents the specific productivity (Qp) of a single clone (picograms/cell/day, pcd); the mean values and SD bars for each group are also shown.

FIG. 12C shows stability of production of trastuzumab in the top producer clone TrasSB-6 during 3 months of continuous culture in selective medium. Each column represents the mean specific productivity of TrasSB-6 in triplicate cultures of 10⁶ cells. SEM bars for each group are also shown.

FIG. 12D shows transgene copy numbers of the LC and HC in 15 clones expressing trastuzumab (Tras plus TrasSB sets in panel b shown as a linear regression with respect to specific productivity (pcd). No significant correlation was seen (r²=0.09, p<0.3).

FIG. 12E shows relative mRNA expression levels of the LC and HC of trastuzumab in 15 clones of Tras and TrasSB shown as a linear regression with respect to specific productivity (pcd). mRNA values have been normalized to the respective expression in an independent CHO K1-derived cell line, GSKO-aHer2. An r² value of 0.54 (p<0.002) indicates a substantial correlation, but with some variation due perhaps to translational or post-translational factors.

FIG. 13 shows transgene copy number determined by ddPCR for individual rescue enzymes in the clones from trastuzumab transfection. Five clones from Tras and 10 from TrasSB transection are shown. The rescue enzymes DHODH, UMPS, PAICS and ATIC were linked to the light chain and CTPS1, TYMS, GMPS and IMPDH2 were linked to the heavy chain in the vectors. The mean copy number+/−SD are also shown.

FIG. 14 shows relative mRNA expression of the individual rescue enzymes in the cell clones of trastuzumab (5 clones in Tras and 10 clones in TrasSB). RNA molecule per cell were determined by NanoString. Genes for the rescue enzymes DHODH, UMPS, PAICS and ATIC were linked to the light chain while those for CTPS1, TYMS, GMPS and IMPDH2 genes were linked to the heavy chain in the vectors. The values have been normalized to the expression of the corresponding endogenous genes in an independent CHO K1-derived cell line, GSKO-aHer2. Mean mRNA expression+/−SD for each enzyme in the 15 clones are also shown.

### DETAILED DESCRIPTION

Properties of CHO cells such as production of human-like glycan structures on secreted proteins, adaptability to suspension medium, refractoriness to human viruses, and growth to high densities has resulted in their workhorse status among mammalian hosts to produce protein biopharmaceuticals of high quality and quantity, most of which are monoclonal antibodies (mAb) (Fischer et al. 2015; Lalonde and Durocher, 2017; Rita et al. 2010; Kuo et al. 2018). However, improvements in the productivity of recombinant protein in CHO cells in more time-saving and cost-efficient ways continue to be pursued in academia and industry.

The present disclosure provides a doubly auxotrophic CHO cell line (UA10 cells) deficient in 5-aminoimidazole-4-carboxamide ribonucleotide formyltransferase/IMP cyclohydrolase (Atic) and uridine monophosphate synthase (Umps) steps in the purine and pyrimidine de novo synthetic pathways, respectively. Employment of this cell line in the production of a model antibody, trastuzumab (Herceptin), showed that transfectant clones could be obtained and characterized within two months. Ten of 12 secreted substantial amounts of mAb and the highest of these fully sustained its productivity for at least 3 months of continuous culture in the selection medium. This double auxotroph provides a convenient means of isolating transfectants that carry independent heavy and light mAb chains by co-transfection with 2 rescuing plasmids of heavy and light chain genes and a single-step selection in purine- and pyrimidine-free medium with no use of antibiotics.

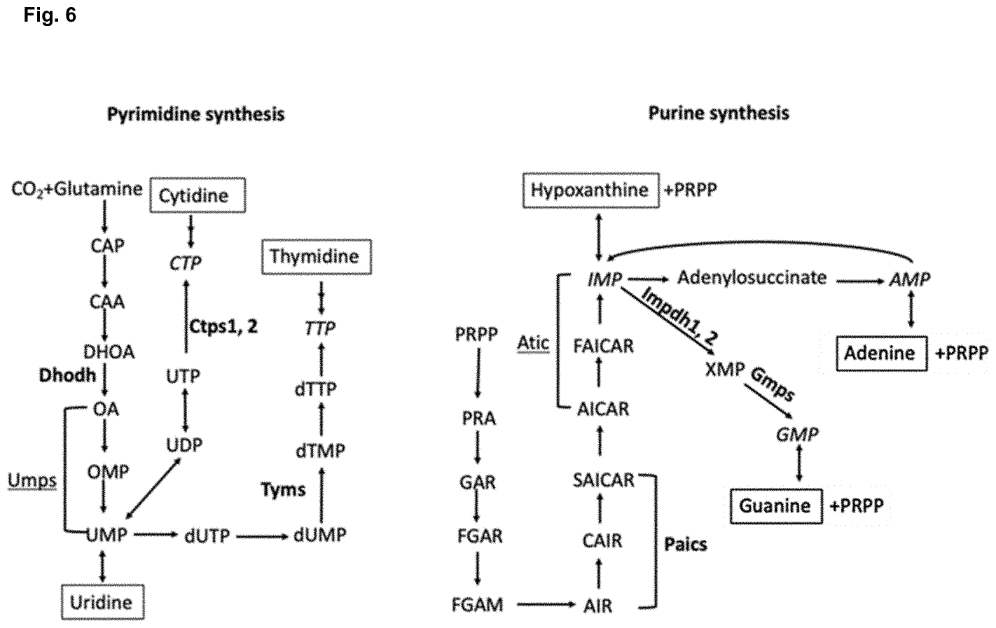

It is believed that a multi-auxotrophic cell line would allow higher order co-transfections and so enhance productivity by guaranteeing an increased copy number of integrated cargo genes. Pyrimidine and purine biosynthetic pathways offer multiple steps as potential targets for additional knockouts and the use of the identical selection of transfectants in commercially available purine- and pyrimidine-free media. The enzymatic steps involved in pyrimidine and purine synthesis are shown in FIG. 6; the details of the pathways have been described in paragraph [0043] above. Starting with CHO derived UA10 cells, we chose to target 6 steps in addition to Umps and Atic, as indicated in FIG. 6. The nutrients uridine, cytidine, thymidine, hypoxanthine, adenine and guanine in medium can be converted to UMP, CTP, TTP, IMP, AMP and GMP, respectively, via salvage pathways and thus should allow growth of such multi-auxotrophs, as shown in FIG. 6.

In the present disclosure, 4 enzymes (DHODH, CTPS1, CTPS2 and TYMS) responsible for 3 steps in the pyrimidine pathway and 4 enzymes (PAICS, IMPDH1, IMPDH2 and GMPS) responsible for 3 steps in the purine pathway were further knocked out as targets in UA10 cells. The newly established cell line (CHO-8A) with 10 enzymes responsible for 8 steps in pyrimidine and purine synthesis, grew well in the medium supplemented with the appropriate nutrients. These cells grew well in the absence of nutrients after transfection with the required ORFs in the form of rescuing genes. The CHO-8A cells provide an effective platform for the flexible and rapid production of highly expressing cell clones of recombinant protein, by which the numbers of different transgenes can be adjusted from 1 to 8 through manipulation of the nutrients in the selective medium without the need of any toxic chemicals. Simultaneous transfection of CHO-8A cells with 8 rescuing plasmids, each carrying a heavy and a light chain gene for trastuzumab, yielded one clone producing more than 80 picograms per cell per day (pcd) and could be isolated within 2 months by screening only tens of colonies. Thus CHO-8A represents a potentially useful host for the rapid isolation of cell lines engineered to produce therapeutic recombinant proteins.

Accordingly, one embodiment of the present disclosure is a multiply auxotrophic cell line that is deficient in (i) at least one gene encoding an enzyme in the de novo pathway for pyrimidine nucleotide synthesis and (ii) at least one gene encoding an enzyme in the de novo pathway for purine nucleotide synthesis.

In some embodiments, the cell line is deficient in at least two genes in the de novo pathway for pyrimidine nucleotide synthesis and/or in the de novo pathway for purine nucleotide synthesis. In some embodiments, the cell line is deficient in two to twenty-three genes in the de novo pathway for pyrimidine nucleotide synthesis and/or in the de novo pathway for purine nucleotide synthesis. In some embodiments, the cell line is deficient in 2, 3, 4, 5, 6, 7, 8, 9, 10, 11, 12 or 13 genes in the de novo pathway for pyrimidine nucleotide synthesis and/or in the de novo pathway for purine nucleotide synthesis.

In some embodiments, the enzymes in the de novo pathway for pyrimidine nucleotide synthesis are selected from uridine monophosphate synthetase (UMPS), dihydroorotate dehydrogenase (DHODH), CTP synthase 1 and 2 (CTPS1/2) and thymidylate synthetase (TYMS), and the enzyme in the de novo pathway for purine nucleotide synthesis is 5-aminoimidazole-4-carboxamide ribonucleotide formyltransferase/IMP cyclohydrolase (ATIC).

In some embodiments, the enzymes in the de novo pathway for pyrimidine nucleotide synthesis are selected from uridine monophosphate synthetase (UMPS), dihydroorotate dehydrogenase (DHODH), CTP synthase 1 and 2 (CTPS1/2) and thymidylate synthetase (TYMS), and the enzymes in the de novo pathway for purine nucleotide synthesis are selected from 5-aminoimidazole-4-carboxamide ribonucleotide formyltransferase/IMP cyclohydrolase (ATIC), phosphoribosylaminoimidazole carboxylase (PAICS) and guanosine monophosphate synthetase (GMPS).

In some embodiments, the enzymes in the de novo pathway for pyrimidine nucleotide synthesis are selected from uridine monophosphate synthetase (UMPS), dihydroorotate dehydrogenase (DHODH), CTP synthase 1 and 2 (CTPS1/2) and thymidylate synthetase (TYMS), and the enzymes in the de novo pathway for purine nucleotide synthesis are selected from 5-aminoimidazole-4-carboxamide ribonucleotide formyltransferase/IMP cyclohydrolase (ATIC), phosphoribosylaminoimidazole carboxylase (PAICS), inosine-5′-monophosphate dehydrogenase 1 and 2 (IMPDH1/2), and guanosine monophosphate synthetase (GMPS).

In some embodiments, the enzymes in the de novo pathway for pyrimidine nucleotide synthesis are selected from uridine monophosphate synthetase (UMPS), dihydroorotate dehydrogenase (DHODH), CTP synthase 1 and 2 (CTPS1/2) and thymidylate synthetase (TYMS), and the enzymes in the de novo pathway for purine nucleotide synthesis are selected from 5-aminoimidazole-4-carboxamide ribonucleotide formyltransferase/IMP cyclohydrolase (ATIC), phosphoribosylaminoimidazole carboxylase (PAICS), adenylosuccinate lyase (ADSL), inosine-5′-monophosphate dehydrogenase 1 and 2 (IMPDH1/2), guanosine monophosphate synthetase (GMPS) and adenylosuccinate synthase and adenylosuccinate synthase like 1 (ADSS/ADSSL1).

In some embodiments, the enzymes in the de novo pathway for pyrimidine nucleotide synthesis are selected from uridine monophosphate synthetase (UMPS), dihydroorotate dehydrogenase (DHODH), CTP synthase 1 and 2 (CTPS1/2) and thymidylate synthetase (TYMS), and the enzymes in the de novo pathway for purine nucleotide synthesis are selected from 5-aminoimidazole-4-carboxamide ribonucleotide formyltransferase/IMP cyclohydrolase (ATIC), phosphoribosylaminoimidazole carboxylase (PAICS), adenylosuccinate lyase (ADSL), inosine-5′-monophosphate dehydrogenase 1 and 2 (IMPDH1/2), guanosine monophosphate synthetase (GMPS), adenylosuccinate synthase and adenylosuccinate synthase like 1 (ADSS/ADSSL1), phosphoribosyl pyrophosphate amidotransferase (PPAT), phosphoribosylglycinamide formyltransferase (GART) and phosphoribosylformylglycinamidine synthase (PFAS).

Another embodiment of the present disclosure is a doubly auxotrophic cell line that is deficient in the gene encoding uridine monophosphate synthetase (UMPS) and the gene encoding 5-aminoimidazole-4-carboxamide ribonucleotide formyltransferase/IMP cyclohydrolase (ATIC).

As used herein, “auxotrophic” or “auxotrophy” refers to the inability of an organism to synthesize a particular organic compound required for its growth.

In some embodiments, the cell line is selected from those commonly used in recombinant proteins production. Non-limiting examples of such cell line include HEK293, HEK293T, BHK21, CHO, CHO/dhfr−, CHO-K1, NS0, Sp2/0-Ag14, and Sp2/0-Ag14-TurboDoma. In some embodiments, the cell line is a CHO cell line. In some embodiments, the cell line is a CHO-K1 cell line.

Another embodiment of the present disclosure is a method for preparing a doubly auxotrophic cell line disclosed herein, comprising the steps of: (a) knocking out a UMPS gene from the genome of the cell line; (b) growing cells from step (a) in a medium containing 5-fluoroorotic acid (5-FOA) and uridine; (c) selecting cells that survive in step (b) and further knocking out an ATIC gene from the genome of the surviving cells; (d) growing cells from step (c) in duplicate in both (i) a medium containing uridine but no hypoxanthine and (ii) a complete medium; and (e) if the cells do not survive in (d-i), collecting their counterparts in (d-ii) as the doubly auxotrophic cells.

In some embodiments, the ATIC and UMPS genes are knocked out by CRISPR-Cas9 vectors. As used herein, “CRISPR-Cas9” refers to a method by which the genomes of living organisms may be edited. By delivering the Cas9 nuclease complexed with a synthetic guide RNA (gRNA) into a cell, the cell's genome can be cut at a desired location, allowing existing genes to be removed and/or new ones added. In some embodiments, other known gene editing methods may be substituted for CRISPR-Cas9, such as, e.g., other engineered nucleases including zinc-finger nucleases (ZFNs) and transcription activator-like effector nucleases (TALENs), viral systems such as rAAV and also transposons.

An additional embodiment of the present disclosure is a method for selecting a cell expressing a protein of interest, comprising the steps of: (a) obtaining a doubly auxotrophic cell line disclosed herein; (b) constructing a first vector carrying a UMPS open reading frame (ORF) and a first coding sequence of the protein of interest; (c) constructing a second vector carrying an ATIC open reading frame (ORF) and a second coding sequence of the protein of interest; (d) transfecting the doubly auxotrophic cells with the first and second vectors; (e) incubating the transfected cells from step (d) in a medium that lacks sources of purines and pyrimidines; and (f) selecting a cell that survives in step (e) as the cell expressing the protein of interest.

In some embodiments, the cell line is selected from the group consisting of HEK293, HEK293T, BHK21, CHO, CHO/dhfr−, CHO-K1, NS0, Sp2/0-Ag14, and Sp2/0-Ag14-TurboDoma. In some embodiments, the cell line is a CHO cell line. In some embodiments, the cell line is a CHO-K1 cell line.

In some embodiments, the first coding sequence is the same as the second coding sequence. In some embodiments, the first coding sequence is different from the second coding sequence.

In some embodiments, the protein of interest is a recombinant protein selected from the group consisting of a decoy receptor, an enzyme used in an enzyme replacement therapy (ERT), a metabolic modulator, and a monoclonal antibody (mAb). Non-limiting examples of a decoy receptor include interleukin 1 receptor type II (IL1R2), decoy receptor 3 (DcR3), VEGFR-1, and ACE-031. Non-limiting examples of an enzyme used in an ERT include agalsidase α, imiglucerase, taliglucerase α, velaglucerase α, alglucerase, sebelipase α, laronidase, idursulfase, elosulfase α, galsulfase, alglucosidase α, α-galactosidase A. Non-limiting examples of a metabolic modulator include human growth hormone, human insulin, follicle-stimulating hormone, factor VIII, erythropoietin, granulocyte colony-stimulating factor (G-CSF), insulin-like growth factor 1 (IGFA-1). In some embodiments, the protein of interest is a monoclonal antibody (mAb).

As used herein, “antibody” refers to immunoglobulins and immunoglobulin fragments, whether natural or partially or wholly synthetically, such as recombinantly, produced, including any fragment thereof containing at least a portion of the variable region of the immunoglobulin molecule that retains the binding specificity ability of the full-length immunoglobulin. Hence, an antibody includes any protein having a binding domain that is homologous or substantially homologous to an immunoglobulin antigen-binding domain (antibody combining site). Antibodies include antibody fragments, such as anti-RSV antibody fragments. As used herein, the term antibody, thus, includes synthetic antibodies, recombinantly produced antibodies, multispecific antibodies (e.g., bispecific antibodies), human antibodies, non-human antibodies, humanized antibodies, chimeric antibodies, intrabodies, and antibody fragments, such as, but not limited to, Fab fragments, Fab′ fragments, F(ab′)₂ fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the above. Antibodies provided herein include members of any immunoglobulin type (e.g., IgG, IgM, IgD, IgE, IgA and IgY), any class (e.g. IgG1, IgG2, IgG3, IgG4, IgA1 and IgA2) or subclass (e.g., IgG2a and IgG2b).

As used herein, “monoclonal antibody” refers to a population of identical antibodies, meaning that each individual antibody molecule in a population of monoclonal antibodies is identical to the others. This property is in contrast to that of a polyclonal population of antibodies, which contains antibodies having a plurality of different sequences. Monoclonal antibodies can be produced by a number of well-known methods (Smith et al. (2004) J. Clin. Pathol. 57, 912-917; and Nelson et al., J Clin Pathol (2000), 53, 111-117). For example, monoclonal antibodies can be produced by immortalization of a B cell, for example through fusion with a myeloma cell to generate a hybridoma cell line or by infection of B cells with virus such as EBV. Recombinant technology also can be used to produce antibodies in vitro from clonal populations of host cells by transforming the host cells with plasmids carrying artificial sequences of nucleotides encoding the antibodies.

Another embodiment of the present disclosure is a method for producing a protein of interest, comprising: (a) obtaining a doubly auxotrophic cell line disclosed herein; (b) constructing a first vector carrying a UMPS open reading frame (ORF) and a first coding sequence of the protein of interest; (c) constructing a second vector carrying an ATIC open reading frame (ORF) and a second coding sequence of the protein of interest; (d) transfecting the doubly auxotrophic cells with the first and second vectors; (e) incubating the transfected cells from step (d) in a medium that lacks sources of purines and pyrimidines; (f) selecting a cell that survives in step (e) as the cell expressing the protein of interest; and (g) producing the protein of interest by culturing the cell selected in step (f).

In some embodiments, the cell line is selected from the group consisting of HEK293, HEK293T, BHK21, CHO, CHO/dhfr−, CHO-K1, NS0, Sp2/0-Ag14, and Sp2/0-Ag14-TurboDoma. In some embodiments, the cell line is a CHO cell line. In some embodiments, the cell line is a CHO-K1 cell line.

In some embodiments, the first coding sequence is the same as the second coding sequence. In some embodiments, the first coding sequence is different from the second coding sequence. In some embodiments, the protein of interest is a recombinant protein as disclosed herein. In some embodiments, the protein of interest is a monoclonal antibody (mAb). In some embodiments, the first coding sequence encodes the light chain of the monoclonal antibody and the second coding sequence encodes the heavy chain of the monoclonal antibody. In some embodiments, the first coding sequence encodes the heavy chain of the monoclonal antibody and the second coding sequence encodes the light chain of the monoclonal antibody.

In some embodiments, higher levels of antibody expression can be obtained by varying the ratio of the first and second vectors to produce a more favorable ratio of light to heavy chain expression. In some embodiments, the doubly auxotrophic cells in step (d) are transfected with equal ratio of the first and second vectors. In some embodiments, the doubly auxotrophic cells in step (d) are transfected with unequal ratio of the first and second vectors.

In some embodiments, the UMPS ORF and/or the ATIC ORF are mutated to increase the stringency of selection. In some embodiments, the first and/or second vectors further contain an epigenetic regulatory element to protect transgene expression.

As used herein, an “epigenetic regulatory element” or “epigenetic regulator” is a DNA sequence which may protect transgenes expression levels from being limited by an unfavorable chromatin structure at the integration site. Non-limiting examples of an epigenetic regulatory element include MARs, UCOE, STARs, and combinations thereof. In some embodiments, the epigenetic regulatory element is selected from the group consisting of Human MAR 1-68, Human MAR X-29, Murine MAR S4, Chicken Lysozyme MAR, Human MAR 1-68 Core+flanking region, 4X Core MAR X29, Chicken beta-globin HS4 Insulator, UCOE from the HNRPA2B1-CBX3 locus, STAR Element 7, STAR Element 40, and combinations thereof.

In some embodiments, the protein of interest is a bispecific monoclonal antibody (BsMAb). In some embodiments, i) the first vector is a tricistronic vector and the first coding sequence encodes a heavy chain and a light chain from a first monoclonal antibody; ii) the second vector is a tricistronic vector and the second coding sequence encodes a heavy chain and a light chain from a second monoclonal antibody; and iii) the first monoclonal antibody is different from the second monoclonal antibody.

As used herein, a “bispecific antibody” refers to a class of engineered antibody and antibody-like proteins that, in contrast to ‘regular’ monospecific antibodies, combine two or more different specific antigen binding elements in a single construct. Since bispecific antibodies do not typically occur in nature, they are constructed either chemically or biologically, using techniques such as cell fusion or recombinant DNA technologies.

A further embodiment of the present disclosure is a kit for selecting a cell expressing a protein of interest, comprising: i) a doubly auxotrophic cell line disclosed herein; ii) a first vector carrying a UMPS open reading frame (ORF) and a first coding sequence of the protein of interest; iii) a second vector carrying an ATIC open reading frame (ORF) and a second coding sequence of the protein of interest; iv) a medium that lacks sources of purines and pyrimidines; and v) instructions of use.

In some embodiments, the cell line is selected from the group consisting of HEK293, HEK293T, BHK21, CHO, CHO/dhfr−, CHO-K1, NS0, Sp2/0-Ag14, and Sp2/0-Ag14-TurboDoma. In some embodiments, the cell line is a CHO cell line. In some embodiments, the cell line is a CHO-K1 cell line.

In some embodiments, the first coding sequence is the same as the second coding sequence. In some embodiments, the first coding sequence is different from the second coding sequence. In some embodiments, the protein of interest is a recombinant protein as disclosed herein. In some embodiments, the protein of interest is a monoclonal antibody (mAb).

In some embodiments, a recombinant protein as disclosed herein is produced using the methods of the present disclosure.

In some embodiments, an antibody, such as a monoclonal or bi-specific antibody, is produced using the methods of the present disclosure.

Another embodiment of the present disclosure is a multiply auxotrophic cell line that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), and the gene encoding 5-aminoimidazole-4-carboxamide ribonucleotide formyltransferase/IMP cyclohydrolase (ATIC).

Another embodiment of the present disclosure is a multiply auxotrophic cell line that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), the gene encoding 5-aminoimidazole-4-carboxamide ribonucleotide formyltransferase/IMP cyclohydrolase (ATIC), the gene encoding phosphoribosylaminoimidazole carboxylase (PAICS), and the gene encoding guanosine monophosphate synthetase (GMPS).

Another embodiment of the present disclosure is a multiply auxotrophic cell line that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), the gene encoding 5-aminoimidazole-4-carboxamide ribonucleotide formyltransferase/IMP cyclohydrolase (ATIC), the gene encoding phosphoribosylaminoimidazole carboxylase (PAICS), the genes encoding inosine-5′-monophosphate dehydrogenase 1 and 2 (IMPDH1/2), and the gene encoding guanosine monophosphate synthetase (GMPS).

Another embodiment of the present disclosure is a multiply auxotrophic cell line that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), the gene encoding 5-aminoimidazole-4-carboxamide ribonucleotide formyltransferase/IMP cyclohydrolase (ATIC), the gene encoding phosphoribosylaminoimidazole carboxylase (PAICS), the gene encoding adenylosuccinate lyase (ADSL), the genes encoding inosine-5′-monophosphate dehydrogenase 1 and 2 (IMPDH1/2), the gene encoding guanosine monophosphate synthetase (GMPS), and the genes encoding adenylosuccinate synthase and adenylosuccinate synthase like 1 (ADSS/ADSSL1).

Still another embodiment of the present disclosure is a method for modulating recombinant monoclonal antibody production, comprising: (a) obtaining a multiply auxotrophic cell line disclosed herein; (b) constructing a vector carrying i) the open reading frame (ORF) of a required enzyme; and ii) a coding sequence of the heavy chain of the recombinant monoclonal antibody; or a coding sequence of the light chain of the recombinant monoclonal antibody; (c) constructing another vector according to step (b) with a different required enzyme; (d) repeating step (c) until each of the required enzymes is carried by one vector, and at least one of the vectors carries the coding sequence of the heavy chain or light chain of the recombinant monoclonal antibody or each of the vectors carries the coding sequence of the heavy chain or light chain of the recombinant monoclonal antibody; (e) transfecting the multiply auxotrophic cells with all the vectors; (f) incubating the transfected cells from step (e) in a medium that lacks sources of purines and pyrimidines; (g) selecting a cell that survives in step (f) as the cell expressing the recombinant monoclonal antibody; and (h) producing the recombinant monoclonal antibody by culturing the cell selected in step (g).

In some embodiments, modulating recombinant monoclonal antibody production means controlling said production, including by decreasing or, preferably increasing production of the recombinant monoclonal antibody. In some embodiments, the ratio of vectors carrying the coding sequence of the heavy chain of the recombinant monoclonal antibody and vectors carrying the coding sequence of the light chain of the recombinant monoclonal antibody is designed to optimize the recombinant monoclonal antibody production.

Another embodiment of the present disclosure is a method for producing a multi-subunit protein of interest, comprising: (a) obtaining a multiply auxotrophic cell line disclosed herein; (b) constructing a vector carrying i) the open reading frame (ORF) of a required enzyme; and ii) the coding sequence of a subunit of the protein of interest; (c) constructing another vector according to step (b) with a different required enzyme and the coding sequence of a different subunit of the protein of interest; (d) repeating step (c) until each subunit of the protein of interest is carried by at least one vector carrying a different required enzyme; (e) transfecting the multiply auxotrophic cells with all the vectors; (f) incubating the transfected cells from step (e) in a medium that lacks sources of purines and pyrimidines; (g) selecting a cell that survives in step (f) as the cell expressing the multi-subunit protein of interest; and (h) producing the multi-subunit protein of interest by culturing the cell selected in step (g).

In some embodiments, the multi-subunit protein of interest is a recombinant protein selected from the group consisting of a decoy receptor, an enzyme used in an enzyme replacement therapy (ERT), a metabolic modulator, a trifunctional bispecific antibody, and a monoclonal antibody (mAb). In some embodiments, the multi-subunit protein of interest can be a combination of polypeptides of the signal recognition particle (SRP) subunits, ATP synthase, cleavage and polyadenylation specificity factor (CPSF), a monoclonal antibody, a trifunctional bispecific antibody, and combinations thereof. In some embodiments, the multi-subunit protein of interest is a trifunctional bispecific antibody.

Another embodiment of the present disclosure is a method for optimizing the activity of a protein of interest, comprising: (a) obtaining a multiply auxotrophic cell line disclosed herein that expresses the protein of interest; (b) constructing a vector carrying i) the open reading frame (ORF) of a required enzyme; and ii) the coding sequence of an enzyme that can modulate the activity of the protein of interest; (c) constructing another vector according to step (b) with a different required enzyme and the coding sequence of a different enzyme that can modulate the activity of the protein of interest; (d) repeating step (c) as necessary until each enzyme that can modulate the activity of the protein of interest is carried by at least one vector carrying a different required enzyme; (e) transfecting the multiply auxotrophic cells with all the vectors; (f) incubating the transfected cells from step (e) in a medium that lacks sources of purines and pyrimidines; (g) selecting a cell that survives in step (f) as the cell expressing the protein of interest having desired activity; and (h) producing the protein of interest having desired activity by culturing the cell selected in step (g).

In some embodiments, the enzyme that can modulate the activity of the protein of interest is necessary for catalyzing a step in a pathway to a protein of interest, including, e.g., a recombinant protein, a recombinant monoclonal antibody, or a multi-subunit protein of interest. In some embodiments, the enzyme that can modulate the activity of the protein of interest is involved in a post-translational modification (PTM) of the protein of interest. As used herein, a “post-translational modification” or “PTM” refers to the covalent and generally enzymatic modification of proteins following protein biosynthesis. Non-limiting examples of a post-translational modification or PTM include myristoylation, palmitoylation, isoprenylation, prenylation, glypiatyon, lipoylation, phophopantetheinylation, acylation, acetylation, formylation, alkylation, methylation, amidation, arginylation, polyglutamylation, polyglycylation, butyrylation, gamma-carboxylation, glycosylation, N-linked glycosylation, O-linked glycosylation, polysialylation, malonylation, hydroxylation, iodination, ADP-ribosylation, phosphorylation, adenylylation, uridylylation, propionylation, pyroglutamate formation, S-glutathionylation, S-nitrosylation, S-sulfenylation, S-sulfinylation, S-sulfonylation, succinylation, sulfation, glycation, carbamylation, carbonylation, biotinylation, carbamylation, oxidation, pegylation, spontaneous isopeptide bond formation, ISGylation, SUMOylation, ubiquitination, Neddylation, Pupylation, citrullination, deamidation, eliminylation, disulfide bridge formation, proteolytic cleavage, isoaspartate formation, racemization, protein splicing, and combinations thereof. In some embodiments, the enzyme that can modulate the activity of the protein of interest is a glycosyltransferase or a hydrolase.

In some embodiments, the protein of interest is a recombinant protein as disclosed herein. In some embodiments, enzymes in the de novo pathway for pyrimidine and purine nucleotide synthesis are identified. It is contemplated that all of the methods disclosed herein can use the enzyme(s) as expressly disclosed and in any combination.

A further embodiment of the present disclosure is an octa-auxotrophic cell line that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), the gene encoding 5-aminoimidazole-4-carboxamide ribonucleotide formyltransferase/IMP cyclohydrolase (ATIC), the gene encoding phosphoribosylaminoimidazole carboxylase (PAICS), the genes encoding inosine-5′-monophosphate dehydrogenase 1 and 2 (IMPDH1/2), and the gene encoding guanosine monophosphate synthetase (GMPS).

In some embodiments, the cell line is selected from the group consisting of HEK293, HEK293T, BHK21, CHO, CHO/dhfr−, CHO-K1, NS0, Sp2/0-Ag14, and Sp2/0-Ag14-TurboDoma. In some embodiments, the cell line is a CHO cell line. In some embodiments, the cell line is a CHO-K1 cell line.

Another embodiment of the present disclosure is a method for preparing an octa-auxotrophic cell line disclosed herein, comprising the steps of: (a) obtaining a doubly auxotrophic cell line disclosed herein; (b) knocking out genes DHODH, TYMS, CTPS1 and CTPS2 from the genome of the doubly auxotrophic cell line obtained in step (a); (c) growing colonies of cells from step (b) in duplicate in both (i) a selective medium containing none of thymidine, cytidine, uridine and hypoxanthine and (ii) a complete medium; (d) if the cells do not survive in (c-i), collecting their counterparts in (c-ii) and further confirming the knock-out of DHODH, TYMS, CTPS1 and CTPS2 by DNA-sequencing; (e) selecting cells with confirmed knock-out of DHODH, TYMS, CTPS1 and CTPS2 in step (d) and further knocking out genes GMPS and PAICS from the genome of the selected cells; (f) growing clones of cells from step (e) in duplicate in both (i) a selective medium containing none of thymidine, cytidine, guanine, uridine and hypoxanthine and (ii) a complete medium; (g) if the cells do not survive in (f-i), collecting their counterparts in (f-ii) and further confirming the knock-out of GMPS and PAICS by DNA-sequencing; (h) selecting cells with confirmed knock-out of GMPS and PAICS in step (g) and further knocking out genes IMPDH1 and IMPDH2 from the genome of the selected cells; (i) growing clones of cells from step (h) in a complete medium and confirming the knock-out of IMPDH1 and IMPDH2 by DNA-sequencing; and (j) selecting the cells confirmed in step (i) as the octa-auxotrophic cells.

In some embodiments, the DHODH, TYMS, CTPS1, CTPS2, GMPS, PAICS, IMPDH1 and IMPDH2 genes are knocked out by CRISPR-Cas9 vectors.

Another embodiment of the present disclosure is a method for modulating recombinant monoclonal antibody production, comprising: (a) constructing a vector carrying i) the open reading frame (ORF) of a required enzyme for an octa-auxotrohic cell line; and ii) a coding sequence of the heavy chain of the recombinant monoclonal antibody; or a coding sequence of the light chain of the recombinant monoclonal antibody; (b) constructing another vector according to step (a) with a different required enzyme; (c) repeating step (b) until each of the required enzymes is carried by one vector, and at least one of the vectors carries the coding sequence of the heavy chain or light chain of the recombinant monoclonal antibody or each of the vectors carries the coding sequence of the heavy chain or light chain of the recombinant monoclonal antibody; (d) transfecting the octa-auxotrophic cell line with all the vectors; (e) incubating the transfected cells from step (d) in a medium that lacks sources of purines and pyrimidines; (f) selecting a cell that survives in step (e) as the cell expressing the recombinant monoclonal antibody; and (g) producing the recombinant monoclonal antibody by culturing the cell selected in step (f).

Another embodiment of the present disclosure is a method for modulating recombinant monoclonal antibody production, comprising: (a) constructing a vector carrying i) the open reading frame (ORF) of a required enzyme for an orta-auxotrophic cell line; and ii) a coding sequence of the heavy chain of the recombinant monoclonal antibody; and a coding sequence of the light chain of the recombinant monoclonal antibody; (b) constructing another vector according to step (a) with a different required enzyme; (c) repeating step (b) until each of the required enzymes is carried by one vector; (d) transfecting the octa-auxotrophic cells with all the vectors; (e) incubating the transfected cells from step (d) in a medium that lacks sources of purines and pyrimidines; (f) selecting a cell that survives in step (e) as the cell expressing the recombinant monoclonal antibody; and (g) producing the recombinant monoclonal antibody by culturing the cell selected in step (f).

In some embodiments, the vector constructed in step (b) carries more copies of the coding sequence of the light chain of the recombinant monoclonal antibody than the coding sequence of the heavy chain of the recombinant monoclonal antibody. In some embodiments, the ratio between the copies of the coding sequence of the light chain and the heavy chain is 4 to 1.

Another embodiment of the present disclosure is an octa-auxotrphic cell line made by any process disclosed herein. In some embodiments, the cell line is selected from the group consisting of HEK293, HEK293T, BHK21, CHO, CHO/dhfr−, CHO-K1, NS0, Sp2/0-Ag14, and Sp2/0-Ag14-TurboDoma. In some embodiments, the cell line is a CHO cell line. In some embodiments, the cell line is a CHO-K1 cell line

In some embodiments, the cell lines, compositions, and methods disclosed herein can be used to produce a protein of interest that is effective as an antigen for vaccine production. In some embodiments, the protein of interest is a recombinant protein selected from the group consisting of proteins or protein domains that could serve as antigens to elicit an immune response and so could act as a vaccine. Non-limiting examples of potential antigens include various domains or fragments from the spike protein subunits and the NP protein of SARS Coy 2 virus and the gp120 envelope protein from the HIV virus. In some embodiments, the proteins of interest would be different subunits of a viral or bacterial protein. In some embodiments, a protein of interest produced by the methods disclosed herein is effective as an antigen for vaccine production. In some embodiments, the protein of interest is selected from the group consisting of the spike protein subunits and the NP protein of SARS Coy 2 virus and the gp120 envelope protein from the HIV virus, and combinations thereof. In some embodiments, one or more proteins or protein subunits of interest produced by the methods disclosed herein are effective as an antigen for vaccine production. In some embodiments, the one or more proteins or protein subunits of interest are selected from the group consisting of the spike protein subunits and the NP protein of SARS Coy 2 virus and the gp120 envelope protein from the HIV virus, and combinations thereof.

The disclosure is further illustrated by the following examples, which are offered for illustrative purposes, and are not intended to limit the disclosure in any manner. Those of skill in the art will readily recognize a variety of noncritical parameters, which can be changed or modified to yield essentially the same results.

### EXAMPLES

### Example 1

### A Doubly Auxotrophic CHO-K1 Cell Line for the Production of Recombinant Monoclonal Antibodies

### Methods and Materials

### Cell Culture

CHO-K1 cells (Kao & Puck, 1968) were incubated and maintained in HyClone MEM Alpha Modification with L-glutamine, ribo/deoxyribonucleosides (GE Healthcare Life Sciences) supplemented with 10% fetal bovine serum (FBS, Atlanta Biologicals, Atlanta, Ga.), 100 U/ml penicillin and 100 mg/ml streptomycin, referred to as complete medium, in a humidified 5% CO2 at 37° C. The selection medium was HyClone MEM Alpha Modification without L-Glutamine and Ribo/Deoxyribonucleosides (GE Healthcare Life Sciences) supplemented with 10% dialyzed FBS (Atlanta Biologicals, GA) and 4 mM L-glutamine, referred to as −H−U medium, in the absence/presence of either 100 μM hypoxanthine (+H−U) and 100 μM uridine (−H+U). L-glutamine, hypoxanthine, and uridine were purchased from Sigma-Aldrich.

### Knockout of UMPS and ATIC Genes by CRISPR-Cas9 Vectors in CHO-K1 Cells

Single guide RNAs (gRNAs) of UMPS and ATIC were designed by an online tool CRISPRdirect (Naito, Hino, Bono, & Ui-Tei, 2015); gRNA sequences and genomic targets are listed in Table 1. The All in One pSpCas9 BB-2A-Puro (PX459) v2.0 vector with these gRNA sequences were constructed by GenScript. Before transfection, 3×10⁵ CHO-K1 cells in 2.5 ml of complete medium per well were seeded in 6-well plates and incubated overnight. Plasmids (1 μg) with 3 μl Lipofectamine 2000 (Invitrogen) were incubated at room temperature in 200 μl of OPTI-MEM (Gibco) for 30 min and then added to each well. After 5 h, the medium was replaced with fresh medium and the cells were incubated for an additional 48 h. The cells were then trypsinized and seeded into 96-well dishes at 1 cell per well in complete medium containing 5-fluoroorotic acid (5-FOA, Zymo Research) at 500 μg/ml. After 7 days of selection, surviving colonies were chosen for further analysis. Two confirmed UMPS-clones were used for selection of double knockout UMPS-/ATIC-cells. After gRNA treatment and a 48 h expression period as described above, transfected cells were seeded into 96-well dishes at 1 cell per well and resulting single clones were split into two portions: one incubated in complete medium and the other in −H+U medium. Clones that did not survive in −H+U medium were regarded as potential double UMPS-/ATIC-mutants and their counterparts in the complete medium were collected for further analysis.

TABLE 1
CRISPR-Cas9 gRNA targets.

| | Genomic target† | Locus |
|---|---|---|
| UMPS | CAAATTGCAAGCTCAGGGGA | Exon 3 |
| | TCCGTCTCCACTCCGTGTGC | |
| | ACGTTGTCCAAAATGCTGGA | |
| | GATTCTCGAGCAGCAGAAAA | |
| | AAATTGATGCCGAGATGGTG | |
| | GGGAGAGTGAAGAGGTTCAT | |
| | TCAGGAGAATGTTTTCATAG | |
| | CAACTAATCACAATGGTGTT | |
| | CCCCCTCCTGAGAAGAAAGC | |
| | ATGCAAAGAACTCAGCTTTG | |
| | (SEQ ID NO: 1) | |
| ATIC | GGAGAAGACCTGGTGAAGTG | Exon 15 |
| | GAAGGCACTGTTCGAGGAAG | |
| | TCCCTGAGTTACTCACTGAG | |
| | GCAGAGAAGAAGGAATGGGT | |
| | CGACAAATTGAGAGACGTTT | |
| | CTGTCAGCTCCGATGCCTTC | |
| | TTTCCTTTCAGAGATAACGT | |
| | GGACCGAGCCAAAAGA | |
| | (SEQ ID NO: 2) | |

†The sequences of gRNA targets are underlined.

### Construction of Rescue Vectors and Transfection of Doubly Auxotrophic Cells

The rescue vectors for UMPS and ATIC with an Enbrel or Herceptin heavy or Herceptin light chain open reading frames (ORF) were constructed by modifying the vector pIRESneo3 (Clontech). The basic vector contains the human cytomegalovirus (CMV) major immediate early promoter/enhancer followed by a multiple cloning site (MCS), a synthetic intron (IVS), the encephalomyocarditis virus IRES and the bovine growth hormone polyadenylation signal. Briefly, we replaced the neomycin phosphotransferase (NPT) sequence in pIRESneo with UMPS and ATIC ORFs and cloned the Enbrel sequence into the unique NsiI site downstream of the IVS followed by the IRES and the ORF of UMPS or ATIC. UMPS vectors and ATIC vectors (2 μg) together with 10 μl of Lipofectamine 2000 were incubated in 200 μl of OPTI-MEM medium for 30 min and added into 6-well plate wells containing 3×10⁵ cells in complete medium. After 5 hours, the medium was replaced with fresh medium followed by incubation 36 or 48 hours. The cells were then trypsinized and transferred to −H−U medium for selection.

### Cell Staining by Crystal Violet

Cells were stained in fixing/staining solution (0.05% w/v crystal violet in PBS buffer with 1% formaldehyde and 1% methanol) for 20 min and washed by dipping into a bucket of water. The colonies in air-dried dishes were counted and imaged.

### Genomic DNA Extraction and PCR

The genomic DNA was extracted by GenElute™ Mammalian Genomic DNA Miniprep Kits (Sigma-Aldrich) according to the manufacturer's instructions. PCR with GoTaq® Green Master Mix (Promega) was initiated at 95° C. for 10 min followed by 30 cycles at 95° C. for 30 s, 60° C. for 30 s, and 72° C. for 1 min. A final extension at 72° C. for 5 min was included. The amplified PCR products were subjected to electrophoresis at 120V through 2.5% agarose gels for 30 min. The bands were visualized with ethidium bromide and imaged using a ChemiDoc imaging system (Bio-Rad). The primers for UMPS and ATIC gene detection were as follows: UMPS forward: CCTGAAGGTGACTGATGCCA (SEQ ID NO: 3); UMPS reverse: TTTTGAGGCAAGTGGGTGGA (SEQ ID NO: 4); ATIC forward: AGCCCAAGTGATTTCTGGCA (SEQ ID NO: 5); ATIC reverse: TCAGCCTCAAAGGCAGATGG (SEQ ID NO: 6). The purified PCR products were sequenced by GENEWIZ®.

### Determination of Enbrel and Herceptin Secreted in the Medium by Enzyme-Linked Immunosorbent Assay (ELISA)

Enbrel and Herceptin expressing UA10 cells were seeded in 6-well plates at a density of 1×10⁶ cells in −H−U medium. After a 24 h incubation, the medium was transferred to a tube for concentration determination of Enbrel or Herceptin by ELISA. To perform the ELISA assay, 96-well plates were coated with 100 μL of diluted Capture Ab (AffiniPure Goat Anti-Human IgG (H+L), Jackson Labs; 1:500 dilution in carbonate buffer) and incubated at 4° C. overnight. The plate was washed three times with TBST (50 mM Tris buffered saline with 0.05% of TWEEN® 20) buffer followed by addition of 100 μl of medium from Enbrel or Herceptin expressing UA10 cells or standards and incubation for 2 hours at room temperature. After three washes with TBST buffer secondary Ab (100 μl of goat anti-Human IgG Fc Cross Adsorbed, ThermoFisher Scientific; 1:2000 dilution in TBS with 1% BSA) was added and the plate was incubated for 1 h at room temperature before being washed with TBST three times. ABTS substrate solution (100 μl, ThermoFisher Scientific) was added to each well and developed at room temperature for 12 min. The reaction was stopped by adding 100 μl of 20% SDS. The absorbance was recorded on a plate reader at a wavelength of 415 nm.

### Results and Discussion

### Targets for a Double Auxotroph in CHO-K1 Cells

Our aim was to develop a host cell line into which the heavy and light chain of a given antibody could be introduced using two separate vectors, each carrying a selectable marker that could provide one of the missing functions. The use of auxotrophs eliminates the need for antibiotics to maintain selective pressure on transfectants. We excluded amino acid biosynthetic pathways as targets for auxotrophy since most popular media contain all nonessential amino acids and provision of all 20 amino acids might be required for optimum recombinant protein production. We considered pathways for the biosynthesis of nutrients such as pyrimidines, purines, cholesterol, and inositol all of which have been previously disrupted by mutation in CHO cells (Puck & Kao, 1982). Among these pathways, pyrimidine and purine de novo synthesis are attractive since they offer multiple steps as targets and selection of transfectants could be carried out in commercially available media that lack sources of purines and pyrimidines.

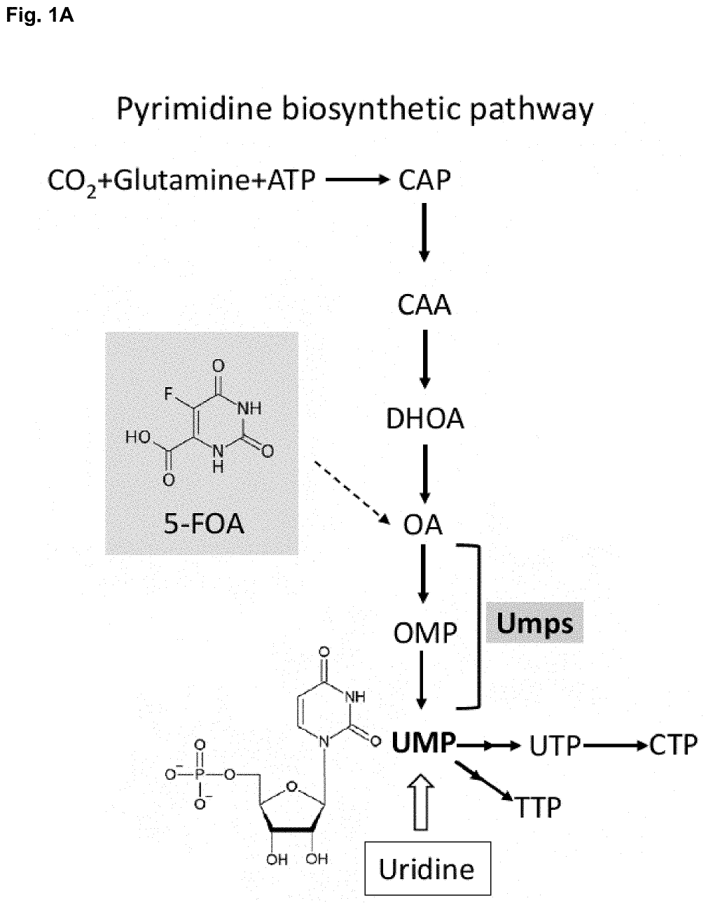

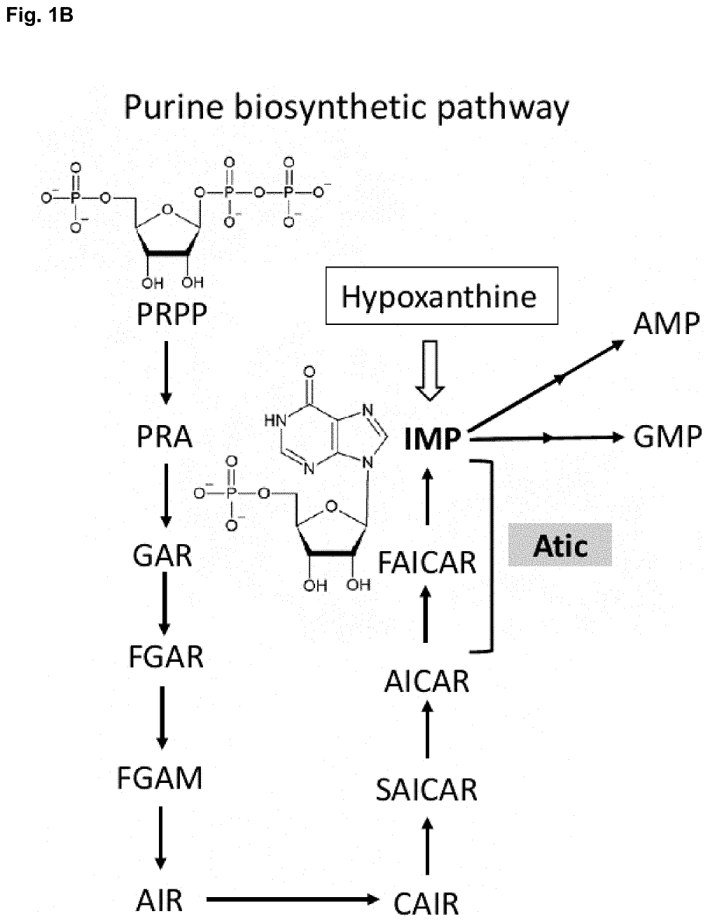

As shown in FIG. 1A, pyrimidines are synthesized starting with carbon dioxide and glutamine to form the intermediate uridine monophosphate (UMP) that is then converted to thymidine triphosphate (TTP), uridine triphosphate (UTP) and cytidine triphosphate (CTP). We chose to target the last step in the formation of UMP, catalyzed by a single bifunctional enzyme, UMP synthetase (Umps) with orotate phosphoribosyltransferase and orotidine-5′-decarboxylase activities. UMPS, was chosen as the knockout target in the pyrimidine pathway because its knockouts can be directly selected by resistance to 5-fluoroorotic acid (5-FOA) in a medium supplemented with uridine. 5-FOA itself is innocuous but it is converted by UMPS into 5-fluoro-UMP that is toxic due to its incorporation into RNA and by its conversion to FUdR, an inhibitor of thymidylate synthetase. As such, it kills cells in the presence of functional Umps. The disruption of Umps eliminates the formation of 5-fluoro-UMP and allows cells to survive in medium containing 5-FOA. 5-FOA is widely used in yeast genetics for the selection for ura3 mutants and has been previously used to select UMPS-mutants of murine erythroleukemic cells (Krooth, Hsiao, & Potvin, 1979).

Inosine monophosphate (IMP), the precursor of adenosine monophosphate (AMP) and guanosine monophosphate (GMP) is synthesized starting from PRPP and glutamine (FIG. 1B). The last 2 enzymatic steps leading to IMP in the de novo purine synthetic pathway are 5-aminoimidazole-4-carboxamide ribonucleotide formyltransferase and IMP cyclohydrolase activities. These two activities are carried out by a single bifunctional enzyme termed Atic. We chose to target the gene for Atic, in part because of its modest size, which was also true for UMPS. Both have open reading frames of less than 2 kb so do not take up much space in rescue vectors. The CHO cells with double knockout of UMPS and ATIC would be auxotrophic and not able to survive in medium without a source of purines and pyrimidines.

### Knockout of UMPS and ATIC by CRISPR-Cas9 in CHO-K1 Cells

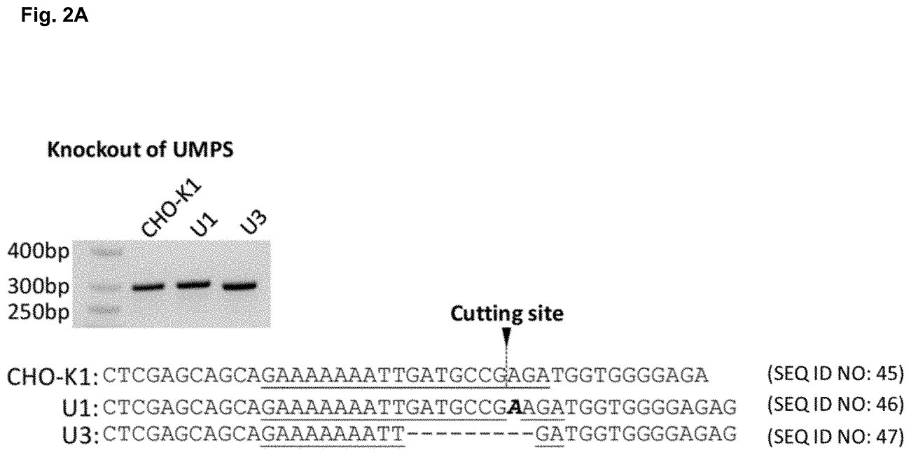

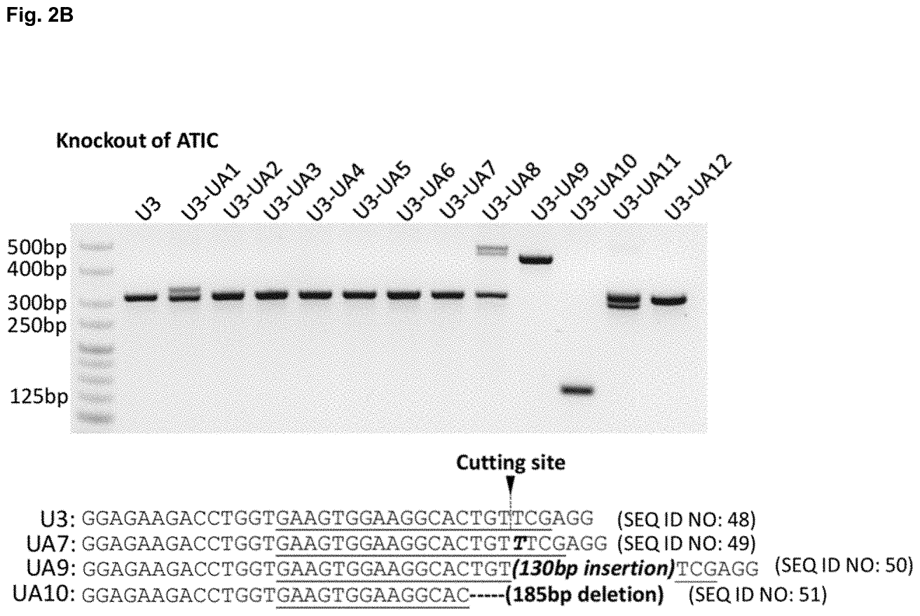

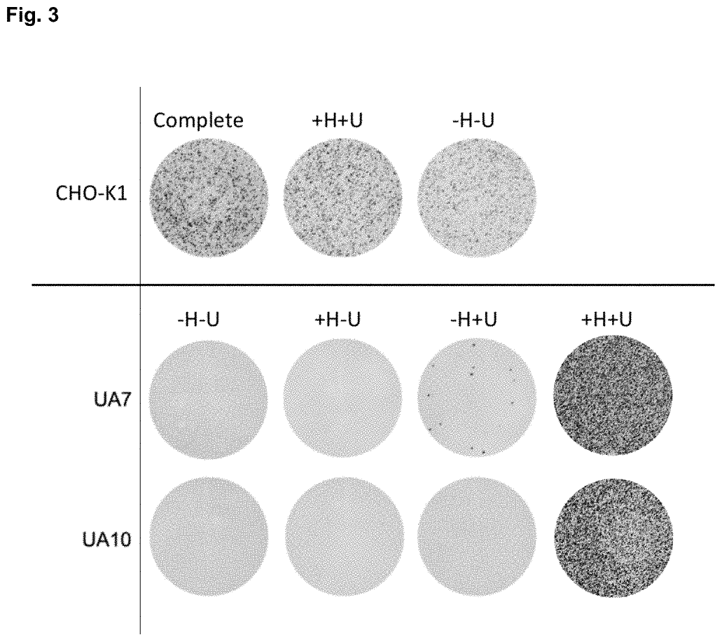

To establish a doubly auxotrophic cell line from CHO-K1 cells, we first used a CRISPR-Cas9 vector with gRNA targeting exon 3 of the UMPS gene. Transfected CHO-K1 cells were selected in the presence of 5-FOA at a concentration of 500 μg/ml at which it killed >99.9% of cells with wild-type UMPS Two surviving clones (U1 and U3) were chosen for further analysis. The DNA sequences surrounding the gRNA targets of UMPS in U1 and U3 were amplified by PCR. Electrophoresis showed that U1 and U3 generated PCR products of a size similar to that of the parental CHO-K1 cells (FIG. 2A). Sanger sequencing showed an A insertion in U1 cells 2 nucleotides preceding the protospacer adjacent motif (PAM) TGG. U3 showed an 8 nucleotides deletion at the same position. Both of these mutations would cause a frame shift starting at amino acid positon 190 in the Umps protein, leading to disruption of at least the orotidine 5′ phosphate decarboxylase activity of this bifunctional enzyme, which runs from position 252 to the carboxyl terminus. Neither U1 nor U3 cells grew in uridine-free (−U) medium and neither gave rise to revertants among 10⁶ cells tested.

We chose U3 cells bearing the 8 bp deletion to knock out the ATIC gene, using a similar CRISPR-Cas9 vector with a gRNA targeting exon 15 of ATIC. Colonies from the transfected cell population (grown in complete medium) were isolated by limited dilution and then split into two portions. One portion was incubated in −H−U medium supplemented with 100 μM uridine (−H+U) compensating for the knockout of the Umps enzyme in U3. The other portion was incubated in complete medium for comparison. The clones that failed to survive in −H+U medium were considered as cells with a double UMPS-/ATIC-mutations. Approximately 80% of the ˜100 clones tested carried ATIC mutations by this criterion, i.e. the cells could not survive in −H+U medium. PCR products of presumed Atic-deficient clones displayed different electrophoretic patterns compared to the parental U3 or to CHO-K1 (FIG. 2B). UA2, UA3, UA4, UA5, UA6, UA7 and UA12 generated sizes similar to that of PCR products of CHO-K1 and U3 cells suggesting missense mutations or very small indels. Sanger sequencing of UA7 showed a T insertion 2 nucleotides preceding the PAM AGG. The PCR products of UA1, UAB, and UA11 have 2 or 3 bands in electrophoresis suggesting complex mutations, heterozygosity or non-clonality. This last could possibly arise if a CRISPR-Cas9 vector had stably integrated into the genome, causing repeated mutations in the ATIC gene. A relatively large insertion or deletion exists in UA9 and UA10 clones, respectively. Sanger sequencing demonstrated that there is a 185 nucleotide deletion starting at 7 nucleotides preceding the PAM AGG in UA10. This deletion starts in exon 15 (of 16) and spans the 5′ splice site at the end of the exon, most likely resulting in the skipping of exon 15, resulting in a deletion of 52 amino acids from the region encoding the 5-aminoimidazole-4-carboxamide ribonucleotide formyltransferase activity of this bifunctional enzyme. In UA9, a 130 nucleotide insertion from the chicken β-actin promoter region of the PX459 vector was inserted into the target site of ATIC near the PAM as shown at the bottom of FIG. 2B. In summary CRISPR-Cas9 was quite efficient in creating the double knockout we sought.

### Growth Characteristics of Double Auxotrophs

We next chose two doubly auxotrophic clones (UA7 and UA10) and challenged them in −H−U media or −H−U media supplemented with either 100 μM uridine (−H+U) or 100 μM hypoxanthine (+H−U). Wild type CHO-K1 cells were incubated in parallel in −H−U, +H+U and complete media. The CHO-K1 cells colonies were of similar size in complete, +H+U and −H−U media although slightly less dense in −H−U medium (FIG. 3), indicating that the UMPS and ATIC genes are functional in these CHO-K1 cells. No colonies were seen when 106UA7 cells were challenged in +H−U medium in 100 mm dishes or when UA10 was challenged in either +H−U or −H+U medium, but 19 colonies appeared when UA7 was challenged in −H+U medium, suggesting that the UA7 ATIC mutation, the insertion of a single T, reverts at an appreciable frequency. That is not the case in UA10 cells with a 185 nt deletion in the ATIC gene. We thus chose the stably mutated UA10 cells as a host cell line for the introduction of recombinant proteins of interest.

### Expression of Enbrel in Permanent Transfectants of the UA10 Double Auxotroph

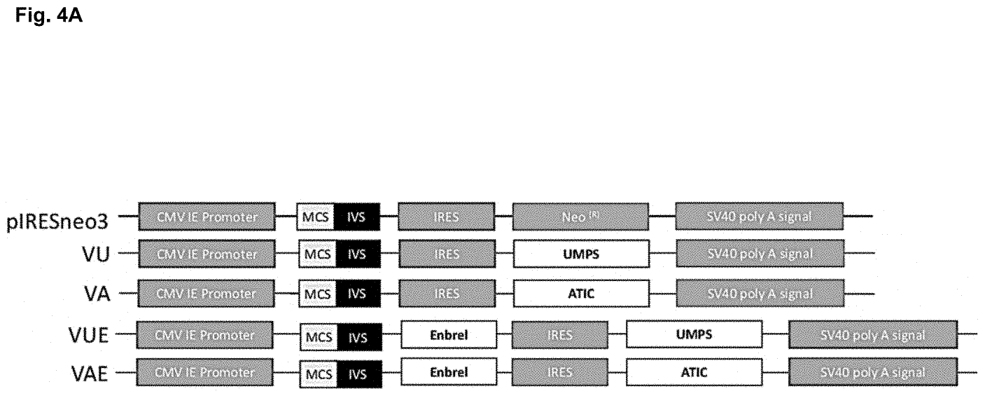

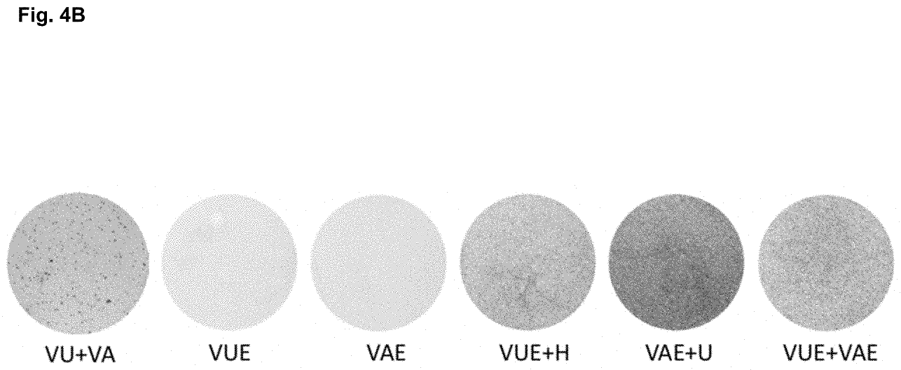

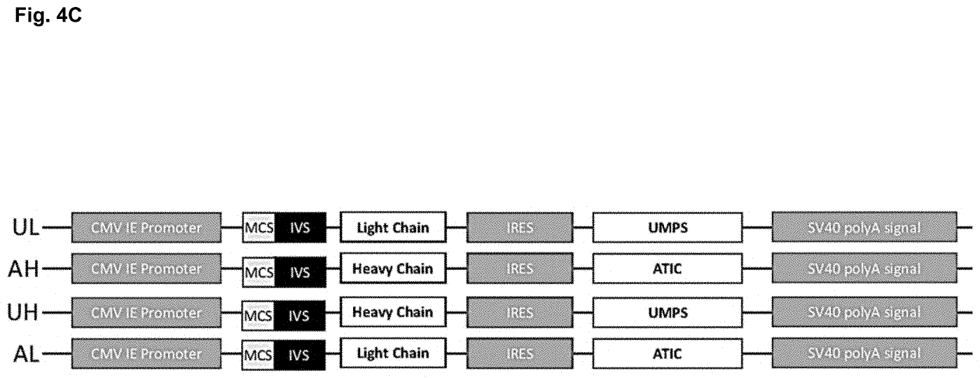

To test the ability of UMPS and ATIC vectors to convert UA10 cells to prototrophy we replaced the Neo gene of a pIRESneo3 vector with a hamster UMPS ORF or ATIC ORF to create VU and VA, respectively (FIG. 4A). As expected, co-transfection of VU and VA into UA10 cells conferred the ability to survive in −H−U selection medium, as shown in FIG. 4B. The ORF for Enbrel was then inserted into An NsiI site downstream of IVS of these vectors These vectors, VUE and VAE, were transfected or co-transfected into UA10 cells. After 2 days, the cells were challenged in −H−U medium, −H+U or +H−U medium. Each vector worked well in terms of colonies formed in the selection media and both vectors were indispensable for cell survival in −H−U medium. Following cotransfection with VUE and VAE, each containing an Enbrel ORF, 6 prototrophic clones were randomly picked and tested for the Enbrel expression. As shown in Table 2, all 6 clones expressed Enbrel, with 4 of 6 producing Enbrel at >5 pg/cell/day (pcd), the highest being 14 pcd.

TABLE 2
Specific productivity of Enbrel in the transfected
single clone cells (pg/cell/day, pcd)

| | |
|---|---|
| Control | VUE + VAE clones |

| UA10 | A | B | C | D | E | F |
|---|---|---|---|---|---|---|
| ND^{‡} | 0.9 ± | 1.5 ± | 6.5 ± | 14 ± | 5.8 ± | 7.9 ± |
| | 0.01 | 0.03 | 0.36 | 4.5 | 0.13 | 0.3 |

^{‡}Not detectable

### Expression of the Heavy and Light Chains of Herceptin in Permanent Transfectants of the UA10 Double Auxotroph

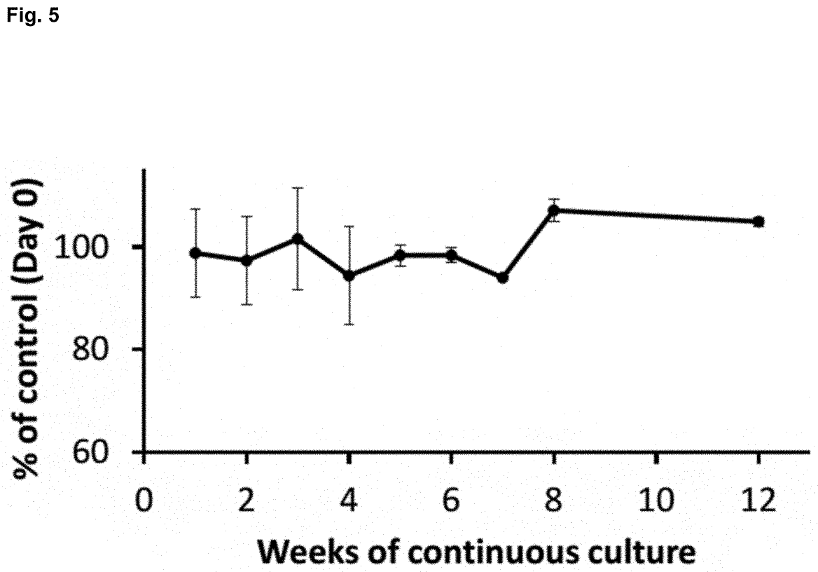

We next extended this type of co-transfection to the introduction of two recombinant proteins, in particular to the formation of a monoclonal antibody (mAb) comprised of a heavy and light chain polypeptides. We chose to use the production of Her-2 antibody (Herceptin) in UA10 cells with bicistronic UMPS and ATIC vectors bearing either a Herceptin heavy or light chain ORF. We constructed vectors with the following four combinations of minigenes: UMPS and heavy chain (UH); ATIC and light chain (AL); UMPS and light chain (UL); ATIC and heavy chain (AH), as shown in FIG. 4C. UL plus AH or UH plus AL, each at 2 ug with 10 ul Lipofectamine 2000 were transfected into UA10 cells. After 2 days of transfection, 3×10⁵ cells were transferred into a 100 mm dish and incubated for 7 days in −H−U medium. Both combinations could successfully rescue UMPS and ATIC expression. Numerous colonies of UA10 transfected with both combinations were formed after 7 days of selection. One thousand transfected cells were also incubated in −H−U medium for 21 days yielding 2 colonies for the UL+AH combination and 11 colonies for the UH+AL combination, suggesting that a longer period of selection (3 weeks) is necessary to obtain permanent transfectant colonies in doubly auxotrophic UA10 cells. The amounts of secreted Herceptin using pooled stably expressing cells and as well as single clones were determined by ELISA. In pooled cells, both the UL+AH and UH+AL combinations exhibited similar expression of Herceptin (5.9 vs. 4.9 pcd). Table 3 lists the productivity values of the single clones. Randomly picked single clones (12 for UL+AH and 12 for UH+AL) all expressed Herceptin, with the highest productivity being 6.6 pcd for clone G of the UH+AL combination. Clone G was chosen for analysis of the stability of productivity. The cells were continuously incubated in −H−U medium for 3 months, during which the Herceptin expression was determined at regular intervals. The results in FIG. 5 demonstrated that clone G expressed Herceptin at a level above 6 pcd with no significant decrease observed over 3 months and 22 passages.

TABLE 3
Specific productivity of Herceptin in the transfected
single clone cells (pg/cell/day, pcd). (A, ATIC gene;
U, UMPS gene; H, heavy chain gene; L, light chain gene)

| Clones | A | B | C | D | E |
|---|---|---|---|---|---|
| AH + UL vectors | 3.6 | 3.4 | 0.8 | 4.3 | 4.2 |
| UH + AL vectors | 0.4 | 4.1 | 4.1 | 4.2 | 4.9 |
| | F | G | H | I | J |
| AH + UL vectors | 0.5 | 2.9 | 5.7 | 2.8 | 3.3 |
| UH + AL vectors | 4.3 | 6.6 | 2.2 | 1.7 | 5.9 |

### Other Target Enzymes in the De Novo Purine/Pyrimidine Synthesis Pathways

There are many additional enzymatic steps in the pyr/pur pathways that could be targeted in the same way as was used for obtaining the Umps and Atic knockouts. These are described in Example 2.

This application could also be used to incorporate all the components of multi-subunit proteins of interest such as trifunctional bispecific antibodies (Shatz et al. 2016) in which 2 different light chains and 2 different heavy chains are being produced in the same cell with or without mutations that favor heterodimer formation. These numbers could be increased to generate many different bispecificities for screening purposes.

The ability to form multisubunit proteins in single cells would also facilitate structural studies of complex proteins for research purposes (e.g., ATP synthase, Cleavage and polyadenylation specificity factor (CPSF)).

Furthermore, multi-KO cells, particularly multi-KO CHO-K1 cells, would be valuable for the introduction of multiple different enzymes that could optimize the activity of therapeutic proteins, For instance, the glycosylation pattern of a protein of interest could be manipulated or modulated by the addition and/or over production of up to 10 different glycosyltransferase and hydrolases (Moremen et al. 2018).

Other enzymes in the purine synthesis pathway including phosphoribosyl pyrophosphate amidotransferase (PPAT), phosphoribosylglycinamide formyltransferase (GART) and phosphoribosylformylglycinamidine synthase (PFAS) may be targeted for knockout to further increase the deficient steps of the synthesis in the multi-KO cells. The other enzymes in the pyrimidine synthesis pathway like the trifunctional enzyme of carbamoyl-phosphate synthetase 2, aspartate transcarbamylase, and dihydroorotase (CAD), kinases, ribonucleotide reductases and phosphohydrolases are not desirable targets for knockouts based on the following reasons: I) CAD is a relatively large protein and has a CDS of 6700 nt rendering it difficult to be transfected with a target gene in a vector; II) kinases, ribonucleotide reductases and phosphohydrolases all have other important physiological and pathological functions in living cells and their knockout may complicate the phenotype of the cells or even be lethal.

### Discussion

In the present study, we established a doubly auxotrophic CHO cell line (UA10) with UMPS and ATIC knocked out by CRISPR-Cas9, causing a disruption of purine and pyrimidine de novo synthesis. The survival of the cells depended on the exogenous provision of a source of purines and pyrimidines or rescued expression of the enzymes. Rescued expression of UMPS and ATIC positioned downstream of an RES in a vector with ORFs of Enbrel or Herceptin light and heavy chains showed that the cells surviving in purine- and pyrimidine-free selection medium expressed Enbrel and Herceptin at a relatively high level without amplification. Stably expressing cell clones could be obtained within 2 months without the use of any toxic materials employed in the medium. Almost all of the randomly picked clones (total of 24) expressed Herceptin within a limited window (20 of 24 were between 2 and 6 pcd). The clone with the highest productivity sustained its capacity for at least 3 months of culture in commercially available selection medium. Taken together, these features suggest that UA10 is a promising CHO host cell line for recombinant protein production and warrants further optimization with targeted and/or systemic engineering.

Two essential enzymes in the purine and pyrimidine de novo synthesis pathways were knocked out. One is UMPS encoding the counterpart of orotidine-5′-monophosphate decarboxylase (ODCase) encoded by the ura3 gene in yeast for which 5-FOA has been widely used in the selection of ura3− cells (Ko, Nishihama, & Pringle, 2008). We employed this drug in a positive selection for knockout of UMPS by Crispr-Cas9 in CHO-K1 cells. The cells that lost UMPS died in uridine-free medium and selectively survived in a medium containing 5-FOA, constituting a useful bi-directional selection for and against UMPS-cells. 5-FOA selection has been successfully used in murine erythroleukemic cells (Krooth et al., 1979) and now in CHO-K1 cells; hence it may be applicable for the selection of UMPS-mutants in most other mammalian cells. A previous study reported that knockout of the ATIC gene in Hela cells induced the accumulation of its substrate AICAR in growth medium (Baresova et al., 2016); AICAR has been demonstrated to be toxic in yeast (Rebora, Laloo, & Daignan-Fornier, 2005). In CHO cells, however, cells with the double knockout of UMPS and ATIC grew well in complete medium. Thus no growth inhibition that could be attributed to the accumulation of AICAR in these experiments was seen either in the ATIC mutants or in their rescued derivatives.

In a previous study, the authors obtained Herceptin expression by co-transfection of the heavy and light chain separately borne on two vectors where each vector carried NPT as a selection marker. The productivity of the pooled transfected cells was only 0.02 pcd and only 40% of randomly picked clones expressed a detectable level of the antibody. When tricistronic vectors carrying both the heavy and light chain and a mutated NPT with reduced activity were used, this percentage increased to 70% and average productivity increased to 4.73 pcd (Ho et al., 2012). In this case, higher levels of antibody expression were obtained by manipulation of the vector design to produce a more favorable ratio of light to heavy chain expression. Using the double auxotroph described here such ratio manipulations could be more easily achieved by simply varying the ratio of the UMPS and ATIC vectors coupled with transposable elements that yield multiple integrations (Balasubramanian, Rajendra, Baldi, Hacker, & Wurm, 2016).

In addition to offering an opportunity to easily manipulate light and heavy chain ratios, the use of a double selection with equal vector inputs may have some advantage. Every one of the 24 transfectant clones tested expressed Herceptin and 80% secreted the antibody within a narrow range. This consistency provides a reproducible baseline for further optimization, such as using mutated UMPS and ATIC to increase the stringency of selection, addition of epigenetic regulator elements in vectors and targeting the transgene to highly transcriptionally active chromatin regions (Lalonde & Durocher, 2017).

Doubly auxotrophic CHO cells could also be used together with tricistronic vectors to easily select for transfectants that synthesize 2 different heavy and 2 light chains to form bispecific antibodies, an emerging class of reagents used to increase specificity and avidity of mAbs (Runcie, Budman, John, & Seetharamu, 2018).

One concern in the production of recombinant protein in CHO cell transfectants is the possible instability of expression of the protein of interest. The major causes for such instability include the loss of transgene copies due to genome rearrangement, epigenetic silencing, and post-transcriptional effects (Moritz, Woltering, Becker, & Gopfert, 2016). However, we saw no sign of instability in the clonal production of Herceptin over a 3 month period of continuous cultivation in selective medium, where this selective medium was a conventional medium containing no toxic agents. In contrast, a decrease in production has been seen in recombinant clones isolated using the popular DHFR and GS selection systems (Chusainow et al., 2009; Costa et al., 2012; Noh, Shin, & Lee, 2018). It remains to be seen what underlies this difference and whether it constitutes a general advantage of this host and vector system.

In summary, the availability of a CHO cell line with 2 selective markers and a simple selective medium without any toxic materials provides flexibility in vector design for rapid and efficient isolation of high productivity clones including those synthesizing multiple polypeptides. The UA10 cell line is thus a promising host for the stable production of recombinant proteins of therapeutic value.

### Example 2

### An Octa-Auxotroph of CHO Cells for Cell Engineering

### Materials and Methods

### Cell Culture

UA10 cells (see Example 1, also Zhang et al. 2020) were incubated and maintained in HyClone MEM Alpha Modification with L-glutamine, ribo/deoxyribonucleosides (GE Healthcare Life Sciences) supplemented with 10% fetal bovine serum (FBS, Atlanta Biologicals, Atlanta, Ga.), 100 U/ml penicillin and 100 μg/ml streptomycin, referred to as complete medium, in a humidified 5% CO2 at 37° C. CHO-8A cells were incubated and maintained under the same conditions except in the complete medium supplemented with uridine, cytidine, thymidine, adenine and guanine each at concentration of 100 μM. The selective medium used in current study was HyClone MEM Alpha Modification without L-Glutamine and Ribo/Deoxyribonucleosides (GE Healthcare Life Sciences) supplemented with 10% dialyzed FBS (Atlanta Biologicals, Atlanta, Ga.) and 4 mM L-glutamine. Uridine, cytidine, thymidine, hypoxanthine, adenine and guanine were purchased from Sigma-Aldrich.

### Genomic DNA Extraction and PCR

The genomic DNA was extracted by GenElute™ Mammalian Genomic DNA Miniprep Kits (Sigma-Aldrich) according to the manufacturer's instructions. PCR with Kod Hot Start Master Mix (MilliporeSigma) or Phusion® High-Fidelity DNA Polymerase (New England Biolabs) was performed according to manufacturer's instruction. The amplified PCR products were purified by DNA Clean & Concentrator Kit (Zymo research) or subjected to electrophoresis at 120V through 2.5% agarose gels for 30-40 min. The bands were visualized with ethidium bromide.

### Knockout by Crispr-Cas9 of the Enzymes in the Pyrimidine and Purine Biosynthetic Pathways

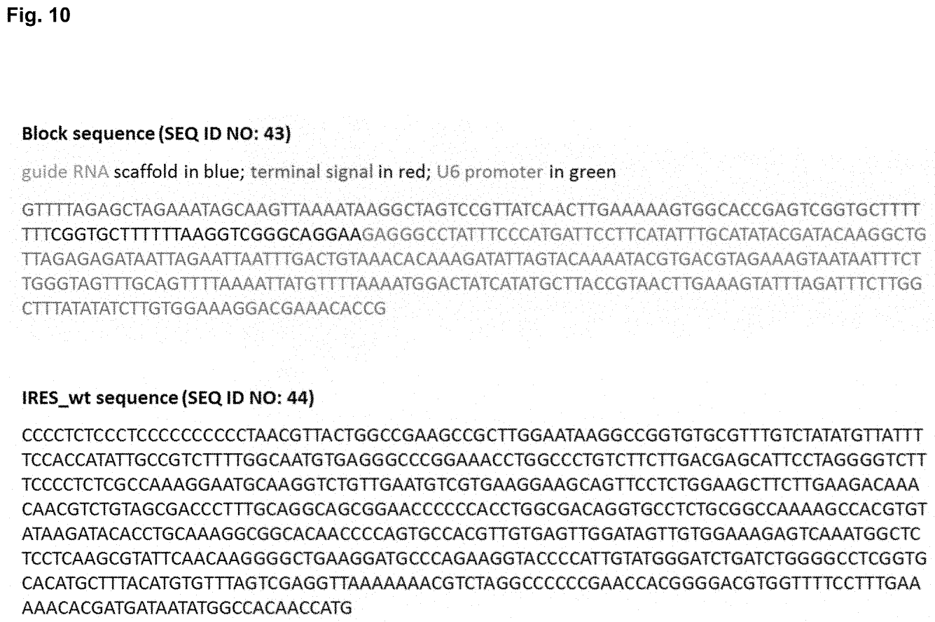

Guide RNAs (gRNAs) of Dhodh, Ctps1, Ctps2, Tyms, Paics, Impdh1, Impdh2 and Gmps were designed by an online tool CRISPRdirect (Naito et al. 2015); gRNA sequences are listed in Table 6. The gRNAs were cloned into the pSpCas9 BB-2A-Puro (PX459) v2.0 vector (Addgene) in single or multiplex forms. The PX459 v2.0 vector was digested by BbsI-HF® (New England Biolabs) and the highest molecular weight product was extracted from the electrophoresis gel and purified using a Gel DNA recovery kit (Zymo Research). A fragment of double stranded DNA (synthesized by IDT) was used as a ligation block to amplify fragments incorporating gRNA sequences by PCR. The block sequence includes the gRNA scaffold, terminal signal and U6 promoter. The sequences used and the primers used to amplify the fragments are listed and PCR reactions are detailed in FIG. 10 and Table 4. We first constructed a vector with multiplex gRNAs of Dhodh, Ctps1, Ctps2 and Tyms. The fragments for these four genes and the longer fragment of digested PX459 v2.0 were ligated using NEBuilder® HiFi DNA Assembly Master Mix (New England Biolabs). The ligation reaction was then transformed into the competent cells followed by isolation of colonies for plasmid extraction (NucleoSpin Plasmid Mini kit, Macherey-Nagel) and Sanger sequencing (Genewiz) to determine correctness of the sequences. The final correct plasmid, referred as vector (1) was a multiplex gRNAs vector possessing 4 gRNAs plus respective sequences of a U6 promoter, gRNA scaffold and termination signal along with all necessary sequences for expression of Cas9. In a similar manner, the gRNA sequences for Gmps and Paics were cloned into the PX459 v2.0 vector, referred as vector (2). The gRNA for Impdh1 and Impdh2 were cloned separately into the PX459 v2.0 vector, creating two separate vectors (vector (3) and (4)) for transfection.

TABLE 4
Primers for construction of
Crispr-Cas9 vectors.

| Gene | Primers |
|---|---|
| Tyms | Forward: |
| | ATCTTGTGGAAAGGACGAAACAC |
| | CGCTGCATGCCGAACACCGACAG |
| | TTTTAGAGCTAGAAATAGCAAGTT |
| | (SEQ ID NO: 7) |
| | Reverse: |
| | TCTACCGGGTACCCTTGTTG |
| | CGGTGTTTCGTCCTTT |
| | (SEQ ID NO: 8) |
| Ctps1 | Forward: |
| | CAACAAGGGTACCCGGTAGAG |
| | TTTTAGAGCTAGAAATAGCAAGTT |
| | (SEQ ID NO: 9) |
| | Reverse: |
| | CATACCGATGTCTGTGTCTT |
| | CGGTGTTTCGTCCTTT |
| | (SEQ ID NO: 10) |
| Ctps2 | Forward: |
| | AAGACACAGACATCGGTATGG |
| | TTTTAGAGCTAGAAATAGCAAGTT |
| | (SEQ ID NO: 11) |
| | Reverse: |
| | TACCGGATTTCGGAATTT |
| | ATCGGTGTTTCGTCCTTT |
| | (SEQ ID NO: 12) |
| Dhodh | Forward: |
| | ATAAATTCCGAAATCCGGTAG |
| | TTTTAGAGCTAGAAATAGCAAGTT |
| | (SEQ ID NO: 13) |
| | Reverse: |
| | ACTTGCTATTTCTAGCTCTACGG |
| | TGTTTCGTCCTTT |
| | (SEQ ID NO: 14) |
| Gmps | Forward: |
| | ATCTTGTGGAAAGGACGAAACAC |
| | CGTCAAGGTTGTAGCGCGCTCTG |
| | TTTTAGAGCTAGAAATAGCAAGTT |
| | (SEQ ID NO: 15) |
| | Reverse: |
| | CTGGGCAGTCGAAGGGATGAC |
| | GGTGTTTCGTCCTTT |
| | (SEQ ID NO: 16) |
| Paics | Forward: |
| | TCATCCCTTCGACTGCCCAGG |
| | TTTTAGAGCTAGAAATAGCAAGTT |
| | (SEQ ID NO: 17) |
| | Reverse: |
| | ACTTGCTATTTCTAGCTCTACG |
| | GTGTTTCGTCCTTT |
| | (SEQ ID NO: 18) |
| Impdh1 | Forward: |
| | ATCTTGTGGAAAGGACGAAACAC |
| | CGGCCACCACCAGCTCGATCCGG |
| | TTTTAGAGCTAGAAATAGCAAGTT |
| | (SEQ ID NO: 19) |
| | Reverse: |
| | ACTTGCTATTTCTAGCTCTACGGT |
| | GTTTCGTCCTTT |
| | (SEQ ID NO: 20) |
| Impdh2 | Forward: |
| | ATCTTGTGGAAAGGACGAAACACC |
| | GGCCAAGAACCTCATAGACGCG |
| | TTTTAGAGCTAGAAATAGCAAGTT |
| | (SEQ ID NO: 21) |
| | Reverse: |
| | ACTTGCTATTTCTAGCTCTACGG |
| | TGTTTCGTCCTTT |
| | (SEQ ID NO: 22) |

Before transfection, 3×105 UA10 cells in 2.5 ml of complete medium per well were seeded in 6-well plates and incubated overnight. For each well, 1 μg of vector (1) with 3 μl of X-tremeGENE™ 9 DNA Transfection Reagent (Roche) were incubated at room temperature in 200μl of OPTI-MEM (Gibco) for 25 min and then added to the well. Two days later, the cells were trypsinized and seeded into 96-well dishes at an average of 1 cell per well in complete medium. Portion of the isolated cell clones were challenged in the selective medium supplemented with various combinations of uridine, hypoxanthine, cytidine and thymidine. The cell clones that required both cytidine and thymidine in addition to uridine and hypoxanthine required by UA10 cells were regarded as having mutated Ctps1, Ctps2 and Tyms. The reserved portion of such cell clones were extracted for genomic DNA and then sent to Genewiz for Sanger sequencing or NGS-based amplicon sequencing. One of the clones with confirmed frame-shift mutations in Dhodh, two Ctps isozymes (Ctps1 and Ctps2), Tyms as well as the previously knocked out Umps and Atic genes were named CHO-5A and used as parental cells for knocking out Paics and Gmps. Vector (2) at 1 μg plus 3μl of the transfection reagent for each well were used for transfection of CHO-5A cells. The cell clones were challenged in a guanine-selective medium (with uridine, cytidine, thymidine, hypoxanthine and without guanine); those enable to grow without guanine were regarded as having mutated Gmps. The genomic mutations in Paics (which was not subject to selection here) and Gmps were detected by Sanger sequencing (Genewiz). We named one such cell clone with both Paics and Gmps mutated as CHO-7A. Lastly, vector (3) and vector (4) were co-transfected into CHO-7A cells to knock out the isozymes Impdh1 and Impdh2. No selective medium was used for these two genes. The genomic DNA was extracted from isolated cell clones and subjected to sequencing (Sanger or NGS-based amplicon sequencing) to detect the mutations. We named the final cell clone as CHO-8A; it carries mutations in the 8 enzymes knocked out here along with the mutated genes for Umps and Atic previously knocked out in UA10 cells. Based on their documented mutational changes and their predicted nutritional responses the CHO-8A cell line is considered to be an octa-auxotroph deficient in 8 steps of pyrimidine and purine biosynthesis.

### Determination of the Growth Rate of CHO-8A

The cell growth rate of CHO-8A was measured in complete medium supplemented with uridine, cytidine, thymidine, adenine and guanine. The cells were seeded in 6 well dishes at 5,000 cells/well. The cell number was counted daily by hemocytometer from day 2 to day 13 of culture. The number of viable (trypan blue excluding) cells per well included both adhered cells and viable cells shed into the medium at the higher densities. The doubling time in the exponential growth phase (days 2-8) was calculated based on the equation: doubling time=ln(2)/k where k is the slope of the best fit line to a semi-ln plot for points from day 2 to day 8 inclusive.

### Rescuing Expression of the Enzymes Knocked Out in CHO-8A Cells

Rescued vectors were constructed as described previously for UA10 (Zhang et al. 2020). The open reading frames (ORFs) of Dhodh, Ctps1, Tyms, Paics, Impdh2 or Gmps were cloned into pIRESneo3 (Clontech), replacing the neomycin phosphotransferase (Neo) sequence to yield 6 new vectors designated pRD, pRC, pRT, pRP, pRI, and pRG, respectively. These 6 plus the 2 rescue vectors already on hand for Umps (pRU) and Atic (pRA), comprised total of the 8 rescue vectors used here.

Various combinations of vectors were transfected into CHO-8A cells that were then challenged in selective medium supplemented with various combinations of the nutrients uridine (U), hypoxanthine (H), cytidine (C), thymidine (T), adenine (A) and guanine (G). Based on the supplemented nutrients, the CHO-8A cells were divided into 9 groups: (1) without the supplemented nutrients; (2) with U, C, A and G; (3) with U, T, A and G; (4) with U, A and G; (5) with T, A and G; (6) with U, T, C and H; (7) with U, T, and C; (8) with A and G; (9) with U, T and C. The CHO-8A cells in each group were transfected with respective rescue vector(s): all 8 vectors for group (1); pRT for group (2); pRC for group (3); pRT and pRC for group (4); pRC, pRU and pRD for group (5); pRI and pRG for group (6); pRA and pRP for group (7); pRT, pRC, pRU and pRD for group (8); pRA, pRP, pRI and pRG for group (9). Before transfection, 3×10⁵ CHO-8A cells in 2.5 ml of complete medium per well were seeded in 6-well plates and incubated overnight. The vectors of 1 μg, 2 μg or 4 μg with 3X μl of X-tremeGENE™ 9 DNA Transfection Reagent were incubated at room temperature in 200μl of OPTI-MEM (Gibco) for 25 min and then added to each well. Two days later, the cells were then trypsinized and transferred into 100 mm dishes in the selective medium with the indicated supplements for each group. Fourteen days later, the cells in the dishes were stained with crystal violet.

Cell Staining with Crystal Violet

Cells were stained in fixing/staining solution (0.05% w/v crystal violet in PBS buffer with 1% formaldehyde and 1% methanol) for 20 min and washed gently by dipping the dishes into a bucket of water. The cells in air-dried dishes were imaged using a Chemidoc™ MP imaging system (Bio-Rad Laboratories or an IX83 inverted microscope ((Olympus)

### Production of Trastuzumab in CHO-8A Cells

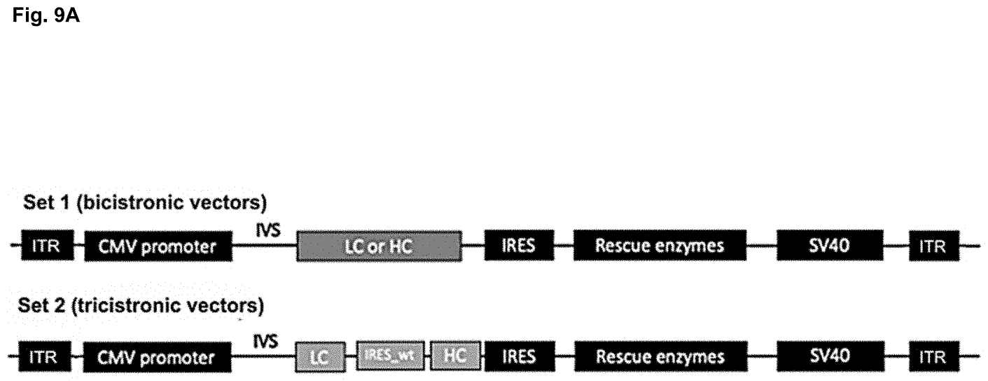

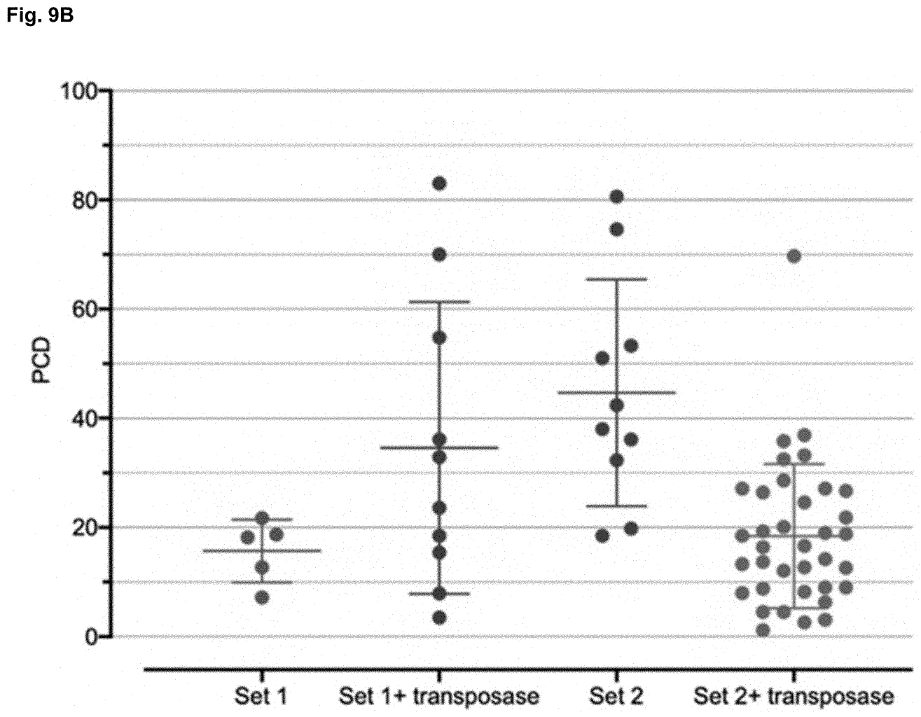

We constructed two sets of vectors expressing the mAb trastuzumab: a bicistronic set (Set 1) expressing the ORFs of a rescue enzyme and either the heavy or light chain of the mAb and a tricistronic set (Set 2) expressing the ORFs of a rescue enzyme and both the heavy or light chain of the mAb (FIG. 9). To construct the bicistronic vectors we first cloned the ORF of the light chain of trastuzumab into the NsiI site of the rescue vectors described in section 2.4 bearing either Dhodh, Umps, Paics or Atic to create vectors pRDL, pRUL, pRPL, pRAL and cloned the ORF of the heavy chain into the NsiI site of the recue vectors bearing Ctps1, Tyms, Impdh2 or Gmps to create pRCH, pRTH, pRIH, and pRGH, referred as pre-Set 1 vectors. The allocation of the L and H chain genes was arbitrary. NsiI-HF® (New England Biolabs) was used for the NsiI digestion. The primers used to amplify the ORFs of light chain and heavy chain from the vectors used previously (see Example 1, also Zhang et al. 2020) are provided in Table 5. The amplified ORFs had tails overlapping the two ends of the Nsi-digested rescue vectors. The ligation was performed using NEBuilder® HiFi DNA Assembly Master Mix to create the 8 pre-Set 1 vectors. The pre-Set 1 vectors were used as precursors to construct the Set 1 vectors to be used for transposase-aided transfection. The fragment including the CMV promoter, the ORF of the light chain or heavy chain, the internal ribosome entry site (IRES), the ORF of the rescue enzyme and the SV40 signal from each Pre-set vector was amplified by PCR (primer sequences are provided in Table 5). The amplified products were then cloned into the PflmI and SphI sites of the vector pSBbi-Bla (Addgene), replacing the longer fragment (to reserve its ITR part, so the promoter, sv40 all used same as in pre-set1). The resulting plasmid contains two tandem inverted terminal repeat (ITR) sequences recognized by the transposase Sleeping Beauty 100X (SB100X) for subsequent insertion into a transfectant genome.

TABLE 5
Primers for construction of A
and B set of vectors.

| | Modules | |
|---|---|---|
| Vector | amplified | Primers |
| Pre- | ORF of | Forward: |
| set | light | TCCCAGGTCCAACTGCAGG |
| | chain | TCGAGCGCCGCCACCATGT |
| | | CCGTG |
| | | (SEQ ID NO: 23) |
| | | Reverse: |
| | | AGAGGGGCGGAATTGGCCGC |
| | | CCTAGATGCATCAGCACTC |
| | | GCCCCGGTTG |
| | | (SEQ ID NO: 24) |
| | ORF of | Forward: |
| | heavy | TCCCAGGTCCAACTGCAGGT |
| | chain | CGAGCGCCGCCACCATGGA |
| | | ATGGT |
| | | (SEQ ID NO: 25) |
| | | Reverse: |
| | | AGAGGGGCGGAATTGGCCGC |
| | | CCTAGATGCATCACTTGCC |
| | | GGGGCTCAGA |
| | | (SEQ ID NO: 26) |
| A set | CMV, | Forward: |
| | ORF | AGCCATAGAGCCCACCGCAT |
| | of | CCCCAGCATGCGATGTACG |
| | light or | GGCCAGATATA |
| | heavy | (SEQ ID NO: 27) |
| | chain, | Reverse: |
| | IRES, | CCCGAGTAGCTAGTTCATGG |
| | ORF of | CAGCCAGCATGTCGACGGTA |
| | rescued | TACAGACATG |
| | enzyme | (SEQ ID NO: 28) |
| | and SV40 | |
| B set | ORF of | Forward: |
| | light | GCGGCCTAGCTAGCGCTTAA |
| | chain | GGCCTGTTAACCGGTGCCG |
| | | CCACCATGTCCGTG |
| | | (SEQ ID NO: 29) |
| | | Reverse: |
| | | GGAGAGGGGCGGAATTGGCC |
| | | GCCCTAGATGTCAGCACTC |
| | | GCCCCGGTTG |
| | | (SEQ ID NO: 30) |
| | IRES_wt | Forward: |
| | | CATCTAGGGCGGCCAATT |
| | | (SEQ ID NO: 31) |
| | | Reverse: |
| | | AGGACCATTCCATGGTTGT |
| | | GGCCATATTATCATCGTGT |
| | | TTTTCAAAGGAAAAC |
| | | (SEQ ID NO: 32) |
| | ORF of | Forward: |
| | heavy | GAAAAACACGATGATAATA |
| | chain | TGGCCACAACCATGGAATG |
| | | GTCCTGGGTGTTCCT |
| | | (SEQ ID NO: 33) |
| | | Reverse: |
| | | AGAGGGGCGGAATTGGCCG |
| | | CCCTAGATGCATTCACTTG |
| | | CCGGGGCTCAGA |
| | | (SEQ ID NO: 34) |

The newly created vectors had tandem transposon ITRs, a trastuzumab light chain or heavy chain and one of the rescue enzymes placed after a weak IRES, and are referred to as Set 1 vectors.

To create the Set 2 vectors that were tricistronic, we first ligated the ORF of the light chain, a fragment of IRES wt (sequence provided in FIG. 10) and the ORF of the heavy chain using the HiFi reaction in which the overlapping sequences were provided by primers (see Table 5) in the PCR reactions. The product was then ligated via the overlapped sequences to the Atic vector of Set 1 digested by AgeI and NsiI to remove the IVS and ORF of the light chain. The obtained tricistronic vector of Atic was cut by AgeI and NsiI to supply the ORF of light chain, the IRES_wt and the ORF of heavy chain for cloning into the same sites in other 7 vectors in Set 1, creating a total of 8 vectors in Set 2.

We transfected the Set 1 or Set 2 vectors (0.5 μg for each one and total of 4 μg) with or without the SB100X transposase-coding Addgene vector pCMV(CAT)T7-SB100 (0.2 μg) into CHO-8A cells seeded at 5×10⁶ per well in 6-well plates the day before transfection. The transfection reagent was 10 μl of X-tremeGENE™ 9-DNA. Two days later, the cells were transferred to 100 mm dishes and incubated in selection medium for 12 days. Cell clones were then isolated and expanded for determination of trastuzumab secretion.

### Determination of Trastuzumab Secreted in the Medium by Enzyme-Linked Immunosorbent Assay (ELISA)

Isolated cell clones described in section 2.6 were seeded in 6-well plates at a density of 1×10⁶ cells/well in selective medium. After a 24 h incubation, the medium was collected for determination of trastuzumab concentration by ELISA. To perform the ELISA assay, 96-well plates were coated with 100 μl of diluted capture antibody (AffiniPure Goat Anti-Human IgG (H+L), Jackson Labs; 1:500 dilution in carbonate buffer) and incubated at 4° C. overnight. The plate was washed three times with TBST (50 mM Tris buffered saline with 0.05% TWEEN® 20) followed by the addition of 100 μl of medium from trastuzumab expressing CHO-8A cells or standards and incubation for 2 h at room temperature. After three washes with TBST buffer, secondary antibody (100 μl of goat anti-Human IgG Fc cross adsorbed, ThermoFisher Scientific; 1:2000 dilution in TBS with 1% BSA) was added and the plate was incubated for 1 h at room temperature before being washed with TBST three times. ABTS substrate solution (100 μl, ThermoFisher Scientific) was added to each well and developed at room temperature for 12 min. The reaction was stopped by adding 100 μl of 20% SDS. The absorbance was recorded on a plate reader at a wavelength of 415 nm.

### Results and Discussion

### Knockout by Crispr-Cas9 of 10 Enzymes Catalyzing 8 Steps in Pyrimidine and Purine Biosynthesis Pathways of CHO Cells

As described in Example 1, we have reported that a doubly auxotrophic cell line (UA10, derived from CHO-K1) with Umps and Atic knocked out facilitated the cotransfection of genes for 2 different recombinant proteins. UA10 cells require the presence of uridine and hypoxanthine in the medium to compensate for these deficiencies. To extend the auxotrophies in UA10 cells and expand subsequent applications, we have now knocked out 8 more enzymes catalyzing 6 other steps in the pyrimidine and purine biosynthesis, 3 steps for each pathway (FIG. 6). We chose Dhodh, Tyms, and the two isozymes Ctps1 and Ctps2 in the pyrimidine pathway and Paics, Gmps and two isozymes in the purine pathway, Impdh1 and Impdh2 as targets. The enzymes and gRNA sequences used in the knockouts are listed in Table 6. Among these enzymes, Tyms and Ctps1/2 act downstream of Umps in pyrimidine synthesis and are involved in conversion of UMP to TTP and CTP, respectively. Dhodh acting immediately upstream of Umps in the pyrimidine pathway is an enzyme that converts DHOA to OA, the substrate for UMPS. Impdh1, Impdh2 and Gmps are the enzymes accounting for the production of GMP from IMP in the purine pathway. Paics is a bifunctional enzyme with both 5-aminoimidazole ribonucleotide carboxylase and 4-(N-succinylcarboxamide)-5-aminoimidazole ribonucleotide synthetase activities, and acts upstream of Atic, as shown in FIG. 6. In a serial knockout manner, we obtained 4 CHO-K1 derived cell lines, each of which require various nutrients in the selective medium for survival (summarized in Table 6).

TABLE 6
The 10 enzymes knocked out by Crispr-Cas9
in the pyrimidine and purine biosynthetic
pathways and the 4 cell lines established.

| | | | | | Cell lines and required exogenous | | | |
|---|---|---|---|---|---|---|---|---|
| | | Enzyme | gRNA | Genotype | nutrients^{a} | | | |
| 1 | | Umps | NA^{b} | 8 bp | UA10b | CHO-5A | CHO-7A | CHO-8A |
| | | | | deletion in | U+H | U+H+C+ | U+H+C+ | U+C+T+ |
| | | | | exon 3 | | T | T | (A or |
| | | | | | | | | H)+G |
| 2 | | Atic | NA^{b} | 185 bp | | | | |
| | | | | deletion | | | | |
| | | | | starting in | | | | |
| | | | | exon 15 | | | | |
| 3 | | Dhodh | ATAAATT | CG | | | | |
| | | | CCGAAAT | insertion | | | | |
| | | | CCGGTA | in exon 3 | | | | |
| | | | (SEQ ID | | | | | |
| | | | NO: 35) | | | | | |
| 4 | | Ctps1 | CAACAAG | A insertion | | | | |
| | | | GGTACCC | in exon 8 | | | | |
| | | | GGTAGA | | | | | |
| | | | (SEQ ID | | | | | |
| | | | NO: 36) | | | | | |
| | | Ctps2 | AAGACAC | T insertion | | | | |
| | | | AGACATC | in exon 4 | | | | |
| | | | GGTATG | | | | | |
| | | | (SEQ ID | | | | | |
| | | | NO: 37) | | | | | |
| 5 | | Tyms | CTGCATG | 2 and 4 | | | | |
| | | | CCGAACA | bp | | | | |
| | | | CCGACA | deletion in | | | | |
| | | | (SEQ ID | exon1 | | | | |
| | | | NO: 38) | | | | | |
| 6 | | Paics | TCATCCC | 5 bp | | | | |
| | | | TTCGACT | deletion in | | | | |
| | | | GCCCAG | exon 6 | | | | |
| | | | (SEQ ID | | | | | |
| | | | NO: 39) | | | | | |
| 7 | Gmps | | TCAAGGT | T and A | | | | |
| | | | TGTAGCG | insertion | | | | |
| | | | CGCTCT | in exon 5 | | | | |
| | | | (SEQ ID | | | | | |
| | | | NO: 40) | | | | | |
| 8 | Impdh1 | | GCCACCA | 5 bp | | | | |
| | | | CCAGCTC | deletion in | | | | |
| | | | GATCCG | exon 7 | | | | |
| | | | (SEQ ID | and 93 bp | | | | |
| | | | NO: 41) | deletions | | | | |
| | | | | in intron 6 | | | | |
| | | | | and exon | | | | |
| | | | | 7 | | | | |
| | Impdh2 | | GCCAAGA | 4 bp | | | | |
| | | | ACCTCAT | deletion | | | | |
| | | | AGACGC | and A | | | | |
| | | | (SEQ ID | insertion | | | | |
| | | | NO: 42) | in exon 9 | | | | |

^{a}U: uridine; H: hypoxanthine; C: cytidine; T: thymidine; A: adenine; G: guanine
^{b}see Example 1, or Zhang et al. 2020.

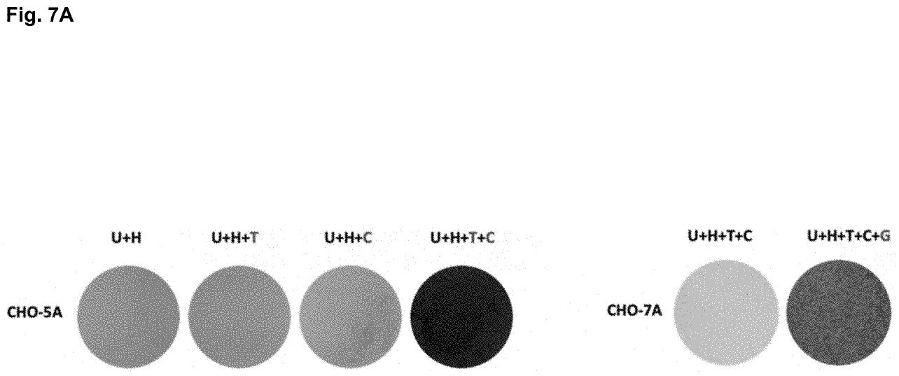

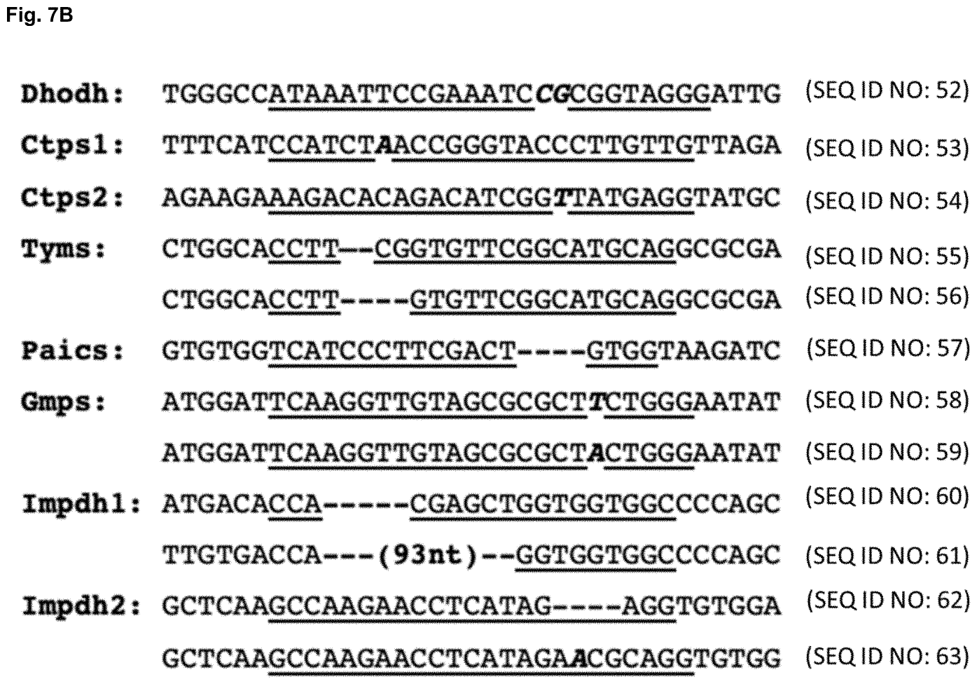

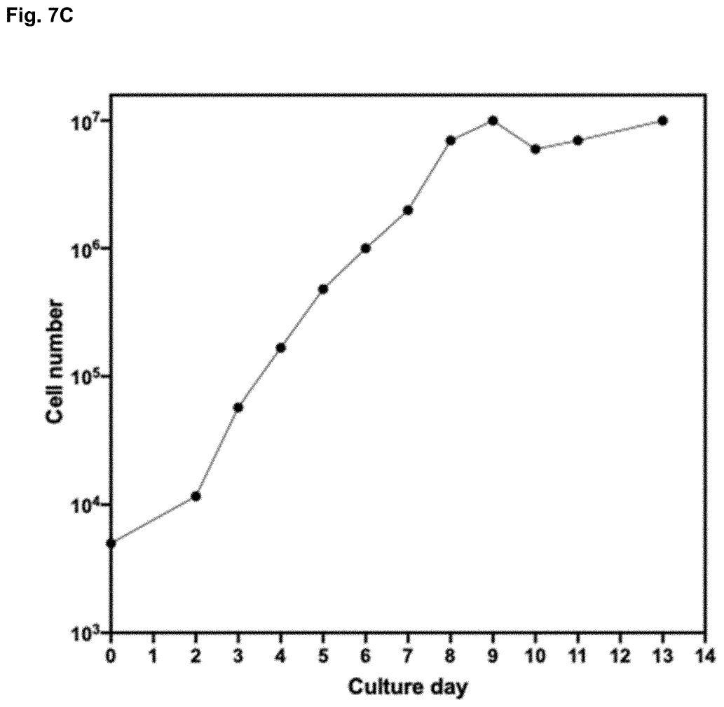

Starting with UA10 cells, we first simultaneously knocked out Dhodh, Tyms, Ctps1 and Ctps2 by multiplex expression of the 4 respective gRNAs in one vector. Colonies that survived in complete medium were screened for their inability to grow in unsupplemented medium. Such clones were then further screened by DNA sequencing for mutations in the targeted genes. The resulted cell clones had the growth phenotypes expected for the disruption of Tyms, Ctpst1 and Ctps2, as they required both thymidine and cytidine in addition to uridine and hypoxanthine for growth, as shown in FIG. 7A. The nutrient-requiring phenotype could not be applied for Dhodh mutants because the UA10 parental cells already require uridine, and so rested on DNA sequencing results. However, its physiological character was confirmed by its rescued expression (see below). From the sequencing results one cell clone was chosen that demonstrated heterozygous mutations in exon 1 of Tyms (2 and 4 bases deletions), homozygous mutations in exon 8 of Ctps1 (A insertion), an exon 4 mutation of Ctps2 (T insertion) and exon 3 of Dhodh (CG insertion), as listed in Table 6 and shown in FIG. 7B. We named this cell clone CHO-5A, having 5 steps disrupted (Dhodh, Umps, Tyms, Ctps1/2, Atic) in both the pyrimidine and purine pathways.

We next knocked out Gmps and Paics in CHO-5A cells, testing for the phenotype of inability to grow in the selective medium containing uridine, hypoxanthine, thymidine and cytidine that compensated all the deficiencies in CHO-5A cells. Cell clones that required the presence of guanine in the selective medium for survival (FIG. 7B) were regarded as those with disrupted GMPS, which is responsible for the conversion of XMP to GMP. Exogenous guanine is converted to GMP in the cell by the salvage enzyme Hgprt. Since Paics is located upstream of Atic whose deficiency requires exogenous hypoxanthine, hypoxanthine in the medium also compensates the Paics deficiency. Hence, no nutrient-requiring test was available for Paics, as was the case for Dhodh. Here again the genotype and phenotype were confirmed by sequencing and rescue experiments. One of the isolated cell clones was subjected to sequencing and had heterozygous T and an A insertions in GMPS exon 5 and a 5 base deletion in PAICS exon 6, as shown in FIG. 7B. We named this cell clone with 7 auxotrophies as CHO-7A.

Continuing with CHO-7A cells, we finally knocked out the genes for Impdh1 and Impdh2. The step catalyzed by these isozymes are located just upstream of Gmps, already knocked out in CHO-7A. Hence, no phenotype verification was performed for these two genes. Cell clones isolated after Crispr-Cas9 treatment revealed heterozygous mutations in both the IMPDH1 and IMPDH2 genes. Impdh1 alleles had a 5 base deletion in exon 7 and a 93 base deletion that extended from intron 6 to base 88 of exon 7. Impdh2 had alleles with a 4 base deletion or an A insertion in exon 9, as shown in FIG. 7B. We named this cell clone as CHO-8A, having 8 auxotrophies.

In summary, we have obtained a new CHO-K1 derived cell line, CHO-8A, with 8 steps disrupted in the pyrimidine (4 steps) and purine (4 steps) biosynthetic pathways. All 10 genes involved in the 8 steps in the pathways had frame shifts as result of insertions or deletions. The deficiencies of Umps, Tyms, Ctps1 and 2, Atic and Gmps have been verified by phenotype testing in the selective media. The deficiencies in the remaining enzymes Dhodh, Paics, and Impdh1 and 2 have been confirmed in rescue experiments described below. CHO-8A cells grew well in medium supplemented with uridine, cytidine, thymidine, adenine and guanine with a doubling time of 16.6 hours, which is comparable to 16.2 hours of parental CHO-K1 cells, as shown in FIG. 7C.

### Rescued Expression of the Enzymes Knocked Out in CHO-8A Cells

We transfected the CHO-8A cells with individual vectors harboring rescue enzymes up to a total of 8. The aims of performing rescue expression experiments were two. Firstly, we wanted to verify the correctness and effectiveness of the rescue enzymes, especially for the isozymes CTPS1, 2 and IMPDH1, 2, as for these isozymes, we provided only one of two isozymes for rescue, i.e., Ctps1 and Impdh2, respectively. Secondly, we wanted to confirm the deficiencies of Dhodh, Paics, Impdh1 and Impdh2 that had not been verifiable in growth phenotype testing.

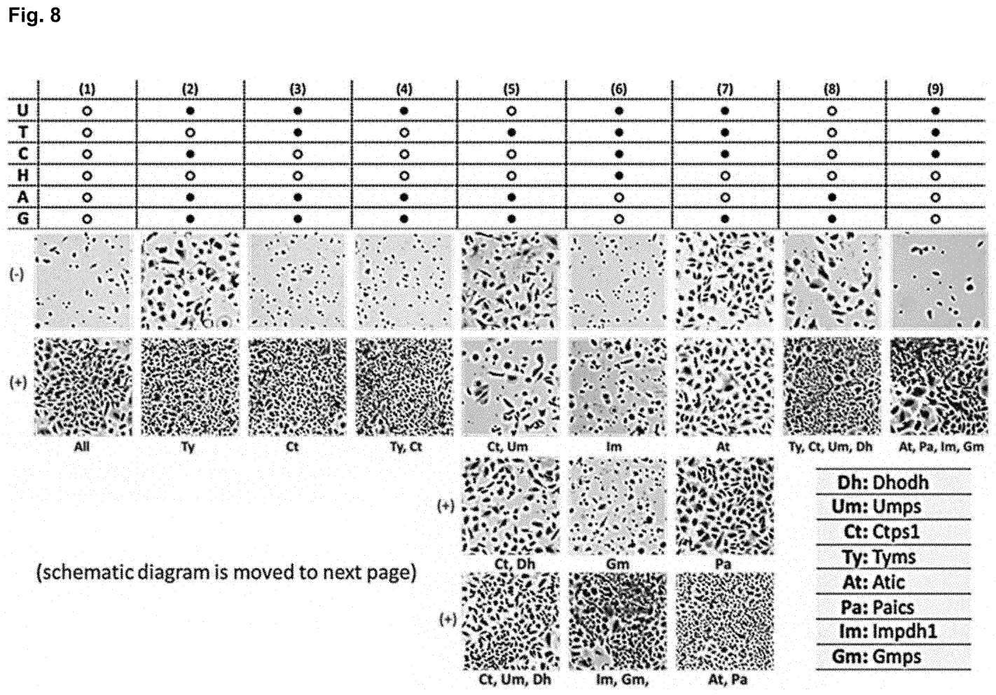

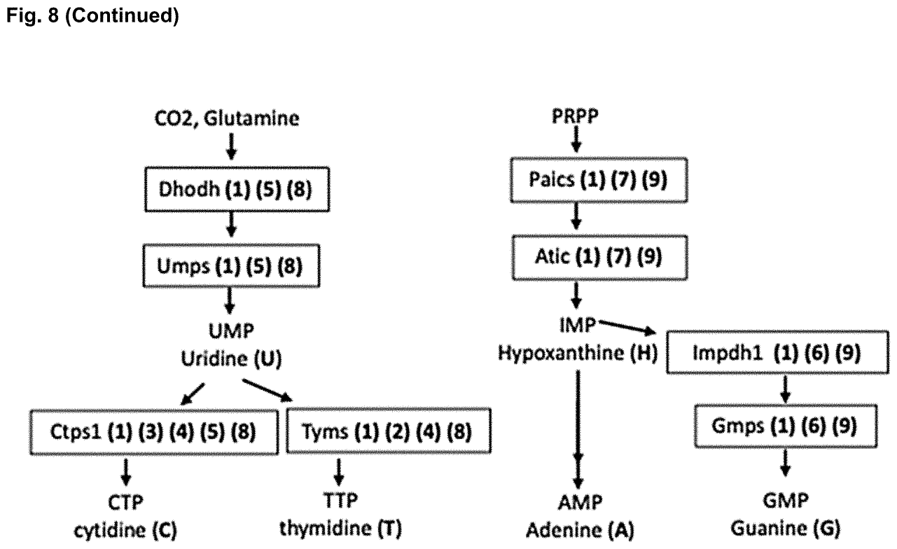

Based on the nutrients provided (U, T, C, H, A and G) in the selective media, we divided the CHO-8A transfections into 9 groups: (1) without any supplemented nutrients; (2) with U, C, A, and G; (3) with U, T, A and G; (4) with U, A and G; (5) with T, A and G; (6) with U, T, C and H; (7) with U, T, C; (8) with A and G; (9) with U, T and C, as shown in FIG. 8. As expected, CHO-8A cells in group (1) all died in the selective medium without the supplemented nutrients while transfection with a mixture of all 8 rescue vectors conferred cell survival. However, this experiment alone does not confirm the presence of all 8 deficiencies or the efficacy of all 8 rescue vectors. In the following steps, we tested the effectiveness of individual vectors as well as the existence of each of the 8 deficiencies. The schematic diagram in the left lower corner of FIG. 8 shows the rescue enzymes predicted to be required for each group.

In group (2), no T (or H) in the selective medium left only TYMS needed for rescue. Without transfection, cells in group (2) did not survive in the selective medium while expression of TYMS rescued the CHO-8A cells. In group (3) CHO-8A cells could not grow in a medium lacking C (and H) but transfection with a CTPS1 vector rescued the cells. In group (4) the medium lacked both C and T (and H) and CHO-8A could not grow, but co-transfection with TYMS and CTPS vectors successfully rescued them. In group 5 we tested for the deficiency and rescue of Dhodh, whose physiological deficiency was not testable by a growth experiment. CHO-8A cells could not grow in a medium with no pyrimidines (first panel in the group (5) column) and could not be rescued by the provision of CTPS plus UMPS alone (second panel) or by CTPS plus DHODH alone (third panel) but were rescued by the mixture of CTPS1 plus UMPS and DHODH vectors. Therefore, all 5 enzymes catalyzing 4 steps in the pyrimidine pathway were disrupted and transfection of the rescue vectors could compensate for these deficiencies.

In group (6) disruption of the activity of IMPDH1/2 and GMPS in the conversion of IMP to GMP were tested. CHO-8A cells could not grow in a medium with H but without A or G (FIG. 8, column 6, first panel). Transfection of either IMPDH2 or GMPS alone could not rescue the CHO-8A cells in this selective medium (column 6, second and third panels), demonstrating that IMPDH2, whose deficiency could not be tested by growth phenotype experiments, had been successfully disrupted. Co-transfection of the 2 genes for these two enzymes did confer the ability of CHO-8A cells to grow.

Similarly, both PAICS and ATIC in group (7) were required for the dense growth of CHO-8A cells in the selective medium without hypoxanthine and adenine which challenged the cells to produce AMP de novo. There were visible tiny colonies (background) in group (7) without transfection or with only one of the enzymes transfected. UA10 cells which has disrupted ATIC were the parental cells for CHO-8A and the disrupted ATIC could completely obstruct the synthesis of AMP with no background growth. The origin of this background might be attributable to the guanine present in this medium; Guanine can be salvaged to GMP by HGPRT and thence to IMP by guanine reductase (Deng et al. 2002). The IMP can then be converted to AMP, as these 2 steps have not been knocked in CHO-8A (FIG. 6). Existence of the background did not jeopardize the conclusion that PAICS activity (not testable by growth phenotype) has been disrupted. We avoided using the combination of group (7) as supplements in application of CHO-8A cells producing recombinant protein (see below). All 5 enzymes in 4 steps of the purine pathway have thus been successfully disrupted and transfection of the rescued vectors can compensate the deficiencies.

We also co-transfected of 4 rescued vectors harboring enzymes in pyrimidine pathway (group (8)) and 4 rescued vectors harboring enzymes in purine pathway (9) to CHO-8A cells challenged in their respective selective media. As expected, CHO-8A cells could survive in the selective media due to rescued expression of the enzymes (FIG. 8).

Collectively, we have successfully knocked out 10 enzymes catalyzing 8 steps in the pyrimidine and purine biosynthesis pathway in CHO-8A cells. Transfection of all 8 rescued enzymes could compensate all the deficiencies (group 1) and no compensatory activities other than those expected were found among the 8 enzymes transfected.

### Flexibility of CHO-8A Cells for the Production of Recombinant Protein

Despite the numerous requirements for the growth of CHO-8A cells, it is not at all necessary to utilize all 8 requirements in a given transfection. By the judicious choice of nutrients one need only to construct any number of vectors from 1 to 8. For example, for the guaranteed permanent integration of 4 genes for different proteins, only 4 vectors need be prepared and 2 nutrients added, as can be seen in Table 7.

TABLE 7
Manipulation of nutrients in the medium to
allow the use of 1 to 8 rescue vectors.

| No. of | | |
|---|---|---|
| rescue | | |
| vectors | | |
| to be used | Rescue gene(s) to be simultaneously used | Supplements |
| 1 | TYMS | ACGU |
| 2 | TYMS, CTPS1 | AGU |
| 3 | TYMS, IMPDH2, GMPS | CHU |
| 4 | TYMS, CTPS1, IMPDH2, GMPS | HU |
| 5 | TYMS, IMPDH2, GMPS, ATIC, PAICS | CU |
| 6 | TYMS, CTPS1, IMPDH2, GMPS, ATIC, | U |
| | PAICS | |
| 7 | CTPS1, IMPDH2, GMPS, ATIC, PAICS, | T |
| | DHODH, UMPS | |
| 8 | TYMS, CTPS1, IMPDH2, GMPS, ATIC, | none |
| | PAICS, DHODH, UMPS | |

Similar manipulations would allow serial rather than simultaneous transfections. Up to 8 serial transfections could be carried out according to the scheme shown in Table 8.

TABLE 8
Scheme for 8 potential serial transfections of CHO-8A cells.

| Transfection No. | Transgene | Supplements |
|---|---|---|
| 1 | TYMS | CGHU |
| 2 | CTPS1 | GHU |
| 3 | UMPS | GHO^{a} |
| 4 | DHODH | GH |
| 5 | GMPS + GPT^{b} | HX^{c} |
| 6 | IMPDH | H |
| 7 | ATIC | AICAR^{d} |
| 8 | PAICS | none |

^{a}O, orotic acid
^{b}GPT, E. coli guanine gpt gene, codes for xanthine phosphoribosyltransferase
^{c}X, xanthine
^{d}AICAR, 5-Aminoimidazole-4-carboxamide-1-β-D-ribofuranoside

The ability to readily introduce up to 8 different genes coding for 8 recombinant proteins represents an efficient way to engineer CHO cells to synthesize a variety of polypeptides after a single transfection, since the integration of all 8 rescue vectors is assured due to selection. Cargoes include multiple antibodies, bispecific antibodies, proteins made up of multiple subunits or multiple enzymes in a pathway. A simpler use would be to use all 8 rescue vectors expressing the same polypeptide(s).

High Level Production of a mAb after a Single Simultaneous Transfection with 8 Vectors

We have used the newly established cell line CHO-8A to produce trastuzumab and compared its productivity to that of the di-auxotrophic host UA10. CHO-8A cells have 6 additional auxotrophies compared to UA10 cells, and thus provide the opportunity to select for transfectants that are guaranteed to have integrated at least one copy of each of 8 rescue vectors. While 8 would be the minimum number of integrations, clones that reflect a distribution of copy numbers would be expected. Thus if the average number of integrations is 10, we might expect transfectants with 80 integrations to be common. We tested this idea using the mAb trastuzumab, which we had previously used to show the utility of di-auxotrophic UA10 cells (Zhang et al. 2020). Almost all of the 24 randomly chosen transfectants of UA10 secreted trastuzumab in a window of 2 to 6 pcd and were able to be created and characterized within 2 months.

To examine the ability of CHO-8A cells to produce trastuzumab we designed two sets of vectors. Set 1 was comprised of bicistronic vectors where the ORF of one trastuzumab light chain or one heavy chain, driven by a CMV promoter, was placed upstream of an IRES driving the ORF of one of the rescue enzymes, as shown in FIG. 9A. In Set 2 the vectors were tricistronic, with an arrangement analogous to Set 1 except that ORFs of light chain and heavy chain were both included in each vector with the light chain driven by the CMV promoter and the heavy chain by a strong IRES (Ho et al. 2012). In this way, the ratio of light chain to heavy chain peptide expression should be approximately 4 to 1, which was shown to be favorable for expression and quality of the antibody (Ho et al. 2012). All of the vectors had one ITR sequence placed before the CMV promoter and one after the SV40 pA sequence, as these are the sites used by Sleeping Beauty transposase for integration. Set 1 or Set 2 of vectors together with or without the transposase vector were transfected into CHO-8A cells; two days later selection in nutrient-free medium was carried out for 10 days.

Cell clones were isolated and incubated for measurement of trastuzumab secreted into the medium by ELISA. While cotransfection with transpose increased the yield of transfectants (data not shown) the distribution of productivities among clones was not noticeably different. As can be seen in FIG. 9B, the majority of clones fell into the range of 10 to 40 pcd, substantially higher than the 2 to 6 pcd range that was previously attained using the di-auxotrophic UA10 cells (see Example 1, also Zhang et al. 2020). More importantly, in 3 of the 4 experimental combinations tested very high producing clones were isolated, the highest being 70, 81 and 83 pcd in 3 experiments. These values are more than 10 times those seen in the highest producing clones using the di-auxotrophic host, more than what might be expected from the 4:1 ratio of selection vectors used. We speculate that the demand for integration of 8 different vectors here operates on a subset of the population that has incorporated large amounts of DNA with a corresponding high copy number of mAb genes. The much lower frequency of transfectant colonies seen when all 8 rescue genes are being selected is consistent with idea of a sub-population of cells enriched for the incorporation of large packages of DNA. Alternative methods of transfection, such as calcium phosphate co-precipitation may produce higher frequencies of such cells. Thus we expect that the methodologies associated with CHO-8A cells are yet to be optimized.

We isolated 5 cell clones from survived cells transfected with the bicistronic vectors without SB100X (Bi-V) and 10 cell clones from those with the transposase (Bi-V-SB100X). As shown in FIG. 9B, all of the cell clones (100%) expressed trastuzumab with an average of productivity for Bi-V clones being 15.7 pcd and for Bi-V-SB100X clones being 34.6 pcd. We previously reported that the productivity of trastuzumab in pooled UA10 cells was 5.4 pcd. Bi-V cell clones which theoretically had at least 4 times copy numbers of the light chain and the heavy chain than UA10 cells in deed produced more trastuzumab, i.e. 2.9-fold increase (15.7 vs. 5.4). SB100X transposase had a potential to increase integration of transgenes into genome (Izsvak et al. 2009), further enhancing the chances and the copy numbers of light and heavy chains integrated in the genome of CHO-8A cells. That was true that more survived cells were observed from transfection of Bi-SB100X group compared those from transfection of Bi-V (data not shown). There were 5 cell clones (50%) with the value of productivity above 30 pcd and 3 cell clones (30%) with the value of productivity above 40 pcd that were high enough to be empirically seen as an indicator for high producer of cell clones.

The isolated 10 cell clones transfected with tricistronic vectors without SB100X had even higher average productivity (44.7 pcd). Among them, 8 cell clones (80%) had the productivity above 30 pcd and 5 cell clones (50%) had the productivity above 40 pcd, suggesting that manipulating the optimal ratio of light chain to heavy chain favored the production of antibody. Unexpectedly, the tricistronic vectors together with SB100X transfected into the CHO-8A cells resulted the isolated cell clones in a lower expression of trastuzumab with an average productivity of 18.4 pcd, compared to 44.7 of cell clones transfected without SB100X. We isolated in total of 36 cell clones in this group, among which 5 cell clones (14%) had the productivity above 30 pcd and only one cell clone (3%) had the productivity above 40 pcd, as shown in FIG. 9B. The possible explanations for this phenomenon include the increased size of tricistronic vector and sub-optimal ratio of SB100X to the transposon for which we did not optimized affecting the efficacy of SB100X.

In summary, we established a new CHO-K1 derived cell line (CHO-8A) deficient in DOHDH, UMPS, CTPS1, CTPS2, TYMS, PAICS, ATIC, IMPDH1, IMPDH2 and GMPS in the pyrimidine and purine de novo synthesis pathways, in which the deficiencies in genotypes and phenotypes were both corroborated. Stepwise expression of the 8 rescued enzymes in various combinations (DOHDH, UMPS, CTPS1, TYMS, PAICS, ATIC, IMPDH2 and GMPS) demonstrated no compensatory activities among them. Application of the CHO-8A cells to produce a model antibody, trastuzumab, manifested favorable properties of CHO-8A cells in production of recombinant proteins: 1) rapid attainment of cell clones permanently expressing 8 or more (using multiplexed vectors) recombinant proteins or subunits within 2 months; 2) ability to achieve high productivity of a single protein; 3) no antibiotics or drugs are needed for selection; 4) flexibility in allocation of transgenes, i.e., a single vector can be used rather 8. In conclusion, CHO-8A cells provide a promising platform for flexible and rapid production of recombinant proteins in highly expressing permanent CHO cell clones.

### Example 3

### A Multi-Auxotrophic CHO Cell Line for the Rapid Isolation of Producers of Diverse or High Levels of Recombinant Proteins

CHO cells are widely used for the high-level production of recombinant proteins. This Example described a multi-auxotrophic mutant of CHO-K1 cells, CHO8A, that is deficient in 8 enzymatic steps in the purine/pyrimidine biosynthetic pathways. Prototrophy was restored by transfections with cDNA-based genes for the 8 missing activities. CHO8A cells permit: 1) selection of transfectant clones that have incorporated genes for 8 or more different polypeptides, suitable for engineering complex proteins or pathways; and 2) the single-step selection of high producers of a particular protein. The latter is achieved by simultaneous use of 8 vectors, each bearing one of the 8 rescue genes and a cargo protein gene. Screening as few as 10 surviving colonies yielded high producers secreting mAbs at 84 pcd or more. CHO8A was isolated by CRISPR-Cas9 knockout of 10 genes in the pathways to pyrimidines (Dhodh, Umps, Ctps1, Ctps2, Tyms) and purines (Paics, Atic, Impdh1, Impdh2 and Gmps).

The manufacture of recombinant protein biologics is most commonly carried out using CHO cells, due to their robustness, human-like glycosylation and ready genetic manipulation. A pervasive challenge in CHO cell line development is to maximize the productivity of a protein of interest, predominantly monoclonal antibodies (mAbs). Approaches to this problem include the use of high expression vectors (Ho et al. 2012), targeted integration of vectors into high expression genomic sites (Sergeeva et al. 2020) and isolation of transfectants carrying high gene copy numbers due to gene amplification (Kingston et al. 2002; Noh et al. 2018) or multiple integrations (Balasubramanian et al. 2016). The interplay of these factors can give rise to great heterogeneity in transfected populations: permanent transfection of CHO cells using a transposon vector has yielded transgene expression levels that span 3 orders of magnitude (Querques et al. 2019). A requirement for the simultaneous integration of numerous independent selective markers, each carried by a different vector, should favor the isolation of transfectants that had integrated a high number of such vectors in order to include at least one of each kind. This strategy has recently been implemented using resistance to multiple antibiotics as selective markers (Balasubramanian et al. 2016). However, antibiotics are limited in number and unwieldy for the maintenance of selective pressure. As an alternative, we created a multi-auxotrophic mutant of CHO-K1 cells that requires the incorporation of 8 “rescue genes” coding for 8 enzymes of nucleotide biosynthesis to permit growth in standard purine/pyrimidine-free medium. A single-step transfection with these 8 transposon-based vectors carrying the heavy or light chain genes of monoclonal antibodies (mAbs) indeed resulted in a high frequency (>10%) of transfectant clones bearing high copy numbers of incorporated vectors and that are capable of pronounced productivity (e.g., 84 picograms per cell per day (pcd)).

### Methods and Materials

### Cell Culture

For routine culturing CHO-K1 cells were grown in HyClone MEM Alpha Modification with L-glutamine and, ribo-/deoxyribonucleosides (GE Healthcare Life Sciences) and supplemented with 10% fetal bovine serum (FBS, Atlanta Biologicals, Atlanta, Ga.), 100 U/ml penicillin and 100 μg/ml streptomycin and incubated in a humidified 5% CO₂ atmosphere at 37° C. CHO8A cells did not grow well in this medium despite its nucleoside content but did grow well if supplemented as well with 100 μM each of uridine, cytidine, thymidine, adenine and guanine. This doubly supplemented medium was used for routine culturing of CHO8A and is designated complete medium. The inability of nucleosides to afford good growth may be due to low or absent levels of guanosine kinase in CHO cells (Hunting et al. 1981). Selective medium was HyClone MEM Alpha modification without L-Glutamine and without ribo/deoxyribonucleosides (GE Healthcare Life Sciences) supplemented with 10% dialyzed FBS (Atlanta Biologicals, Atlanta, Ga.) and 4 mM L-glutamine (Sigma-Aldrich). Various combinations of 100 μM uridine, cytidine, thymidine, hypoxanthine, adenine and guanine (all from Sigma-Aldrich) were used to supplement selective medium as indicated in the text. Hypoxanthine, adenine and guanine were dissolved in 0.1M NaOH, 0.5M HCl and 1M NaOH, respectively, to prepare 100 mM stock solutions.

### CRISPR-Cas9 Knockout of Enzyme Genes in UA10 Cells

Guide RNAs (gRNAs) for Dhodh, Ctps1, Ctps2, Tyms, Paics, Impdh1, Impdh2 and Gmps were designed by the online tool CRISPRdirect (Naito et al. 2015); gRNA sequences are listed in Table 11. The gRNAs were cloned into the pSpCas9 BB-2A-Puro (PX459) v2.0 vector (Addgene) in single or multiplex forms. The PX459 v2.0 vector was digested with BbsI-HF® (New England Biolabs) and the highest molecular weight product was extracted from the electrophoresis gel and purified using a Gel DNA recovery kit (Zymo Research). A fragment of double stranded DNA (synthesized by IDT) was used as a ligation block in the amplification of fragments incorporating gRNA sequences by PCR. The block sequence included the gRNA scaffold, terminal signal and U6 promoter. The sequences and the primers used to amplify the fragments are detailed in FIG. 10 and Table 4. We first constructed a vector with multiplex gRNAs for Dhodh, Ctps1, Ctps2 and Tyms. The fragments for these four genes and the longer fragment of digested PX459 v2.0 were ligated using NEBuilder® HiFi DNA Assembly Master Mix (New England Biolabs). The ligation reaction was then used to transform competent E. coli cells followed by isolation of colonies for plasmid extraction (NucleoSpin Plasmid Mini kit, Macherey-Nagel) and Sanger sequencing (GENEWIZ) to determine correctness of the sequences. The final correct plasmid, referred to as vector (1) was a multiplex gRNAs vector possessing 4 gRNAs plus respective sequences of a U6 promoter, gRNA scaffold and termination signal along with all necessary sequences for expression of Cas9. In a similar manner, the gRNA sequences for Gmps and Paics were cloned into the PX459 v2.0 vector, referred to as vector (2). The gRNAs for Impdh1 and Impdh2 were each cloned separately into the PX459 v2.0 vector, creating vector (3) and vector (4).

TABLE 11
Sequences of gRNA used in
CRISPR-Cas9 knock-out.

| | Gene | gRNA |
|---|---|---|
| 1 | Umps | GAAAAAAATTGATGCCGAGA |
| | (SEQ ID NO: 64) | |
| 2 | Atic | GAAGTGGAAGGCACTGTTCG |
| | (SEQ ID NO: 65) | |
| 3 | Dhodh | ATAAATTCCGAAATCCGGTA |
| | (SEQ ID NO: 66) | |
| | Ctps1 | CAACAAGGGTACCCGGTAGA |
| | (SEQ ID NO: 67) | |
| 4 | Ctps2 | AAGACACAGACATCGGTATG |
| | (SEQ ID NO: 68) | |
| 5 | Tyms | CTGCATGCCGAACACCGACA |
| | (SEQ ID NO: 69) | |
| 6 | Paics | TCATCCCTTCGACTGCCCAG |
| | (SEQ ID NO: 70) | |
| 7 | Gmps | TCAAGGTTGTAGCGCGCTCT |
| | (SEQ ID NO: 71) | |
| 8 | Impdh1 | GCCACCACCAGCTCGATCCG |
| | (SEQ ID NO: 72) | |
| | Impdh2 | GCCAAGAACCTCATAGACGC |
| | (SEQ ID NO: 73) | |

For transfection, 3×10⁵ UA10 cells in 2.5 ml of complete medium per well were seeded in 6-well plates and incubated overnight. For each well, 1 μg of vector (1) with 3 μl of X-tremeGENE™ 9 DNA Transfection Reagent (Roche) were incubated at room temperature in 200 μl of OPTI-MEM (Gibco) for 25 min and then added to the well. Two days later, the cells were trypsinized and seeded into 96-well dishes at an average of 1 cell per well in complete medium. Portions of the isolated cell clones were challenged in the selective medium supplemented with various combinations of uridine, hypoxanthine, cytidine and thymidine. Cell clones that required both cytidine and thymidine, in addition to the uridine and hypoxanthine required by UA10 cells, were regarded as having mutated Ctps1, Ctps2 and Tyms. Reserved portions of such cell clones were extracted for genomic DNA. The targeted genomic sequences were amplified by PCR and then sent to GENEWIZ for Sanger sequencing or NGS-based amplicon sequencing. One clone with confirmed frame-shift mutations in Dhodh, the two CTPS isozymes Ctps1 and Ctps2, and Tyms as well as the previously knocked out Umps and Atic genes was named CHO5A and used as parental cells for knocking out Paics and Gmps. Vector (2) at 1 μg plus 3μl of the transfection reagent for each well were used for transfection of CHO5A cells. Portions of the cell clones were challenged in a guanine-selective medium (with uridine, cytidine, thymidine, hypoxanthine and without guanine); those unable to grow without guanine were regarded as having mutated Gmps. Sanger sequencing (GENEWIZ) was used to detect mutations in Paics (which was not subject to a screening selection here) and Gmps. One such cell clone with both Paics and Gmps mutated (CHO7A) was chosen to carry forward. Lastly, vector (3) and vector (4) were co-transfected into CHO7A cells to knock out Impdh1 and Impdh2. No selective medium was used for these two genes. The genomic DNA was extracted from portions of isolated cell clones and subjected to sequencing (Sanger or NGS-based amplicon sequencing) to detect the mutations. We named the final cell clone CHO8A; it carries mutations in the 8 enzymes knocked out here along with the mutated genes for Umps and Atic previously knocked out in UA10 cells. Based on their documented mutational changes and their predicted nutritional responses the CHO8A cell line is considered to be a multi-auxotroph deficient in 8 steps in pyrimidine and purine biosynthesis.

### Determination of the Growth Rate of CHO8A in Complete Medium

The cell growth rate of CHO8A was measured in the complete medium. Cells were seeded in 6 well dishes at 5,000 cells/well. Viable cell numbers (trypan blue excluding) per well were counted daily by hemocytometer from day 2 to day 13 of culture. The number of viable cells per well included both adherent cells and viable cells shed into the medium at the higher densities. The doubling time in the exponential growth phase (days 2-8) was calculated based on the equation: doubling time=ln(2)/k where k is the slope of the best fit line to a semi-ln plot for points from day 2 to day 8 inclusive.

### Rescuing Expression of the Knocked Out Enzymes in CHO8A Cells

Rescue vectors were constructed as described previously for rescuing the Atic and Umps genes in UA10 (Naito et al. 2015). Briefly, the open reading frames (ORFs) of Dhodh, Ctps1, Tyms, Paics, Impdh2 or Gmps were cloned into pIRESneo3 (Clontech), replacing the neomycin phosphotransferase (Neo) sequences to yield 6 new vectors designated pRD, pRC, pRT, pRP, pRI, and pRG, respectively. These 6 plus the 2 rescue vectors already on hand for Umps (pRU) and Atic (pRA), comprised the total of the 8 rescue vectors used here.

To survey responses to nutrients and rescue vectors, combinations of vectors were transfected into CHO8A cells that were then challenged in selective medium supplemented with various combinations of the nutrients uridine (U), hypoxanthine (H), cytidine (C), thymidine (T), adenine (A) and guanine (G). Based on the supplemented nutrients, the rescue experiments were divided into 8 groups: (a) without any supplemental nutrients; (b) with A and G; (c) with U, T and C; (d) with U, C, A and G; (e) with U, T, A and G; (f) with T, A and G; (g) with U, T, C and H; (h) with U, T, C and G. The CHO8A cells in each group were transfected with corresponding rescue vector(s): all 8 vectors for group (a); pRT for group (2); pRT, pRC, pRU and pRD for group (b); pRA, pRP, pRI and pRG for group (c); pRC for group (d); pRT for group (e); pRC, pRU and pRD for group (f); pRI and pRG for group (g); pRA and pRP for group (h). Before transfection, 3×10⁵ CHO8A cells in 2.5 ml of complete medium per well were seeded into 6-well plates and incubated overnight. Vectors at 1 μg, 2 μg or 4 μg with 3X μl of X-tremeGENE™ 9 DNA Transfection Reagent were incubated at room temperature in 200 μl of OPTI-MEM (Gibco) for 25 min and then added to each well. Two days later, cells from each group were trypsinized and transferred to 100 mm dishes containing selective medium with the indicated supplements (FIG. 2). Fourteen days later, the cells in the dishes were stained with crystal violet.

Cell Staining with Crystal Violet

Cells were stained in fixing/staining solution (0.05% w/v crystal violet in PBS buffer with 1% formaldehyde and 1% methanol) for 20 min and washed gently by dipping the dishes into a bucket of water. The cells in air-dried dishes were imaged using an IX83 inverted microscope (Olympus).

### Production of Trastuzumab and an Anti-SARS-CoV-2 Antibody (9-24)

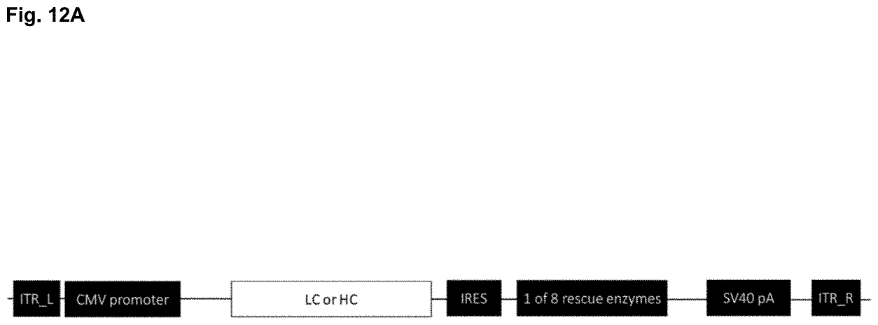

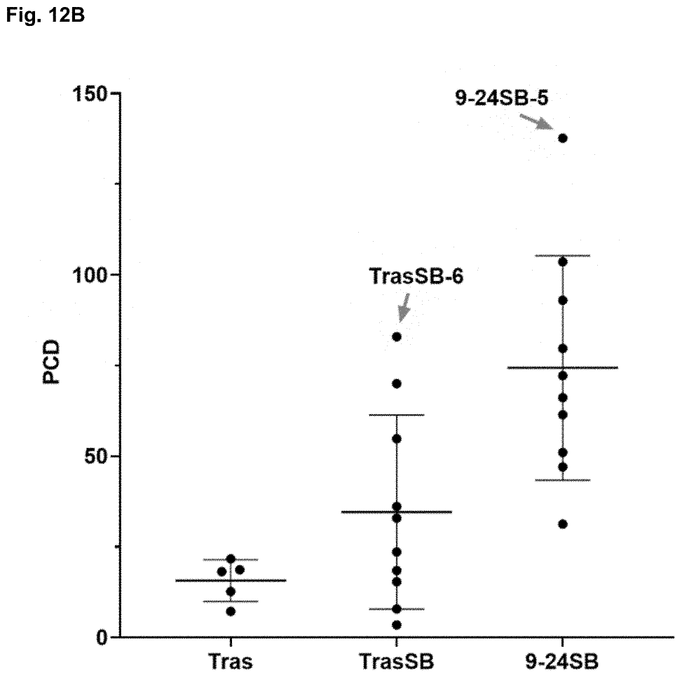

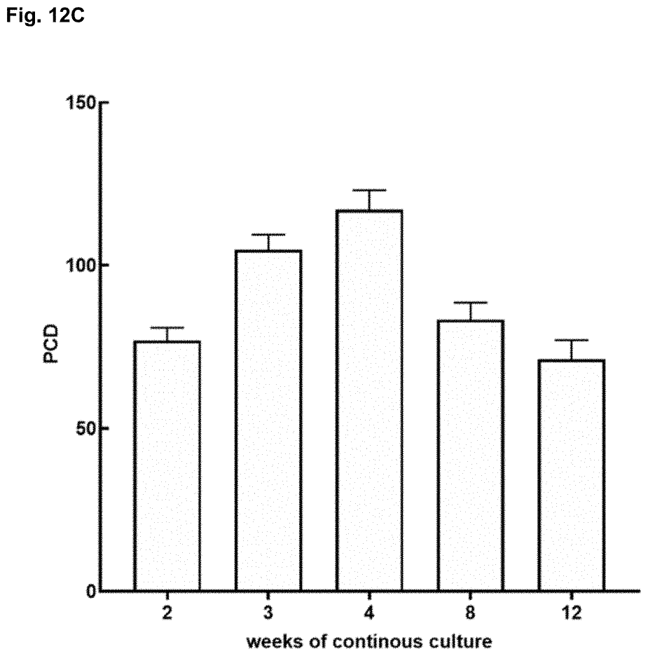

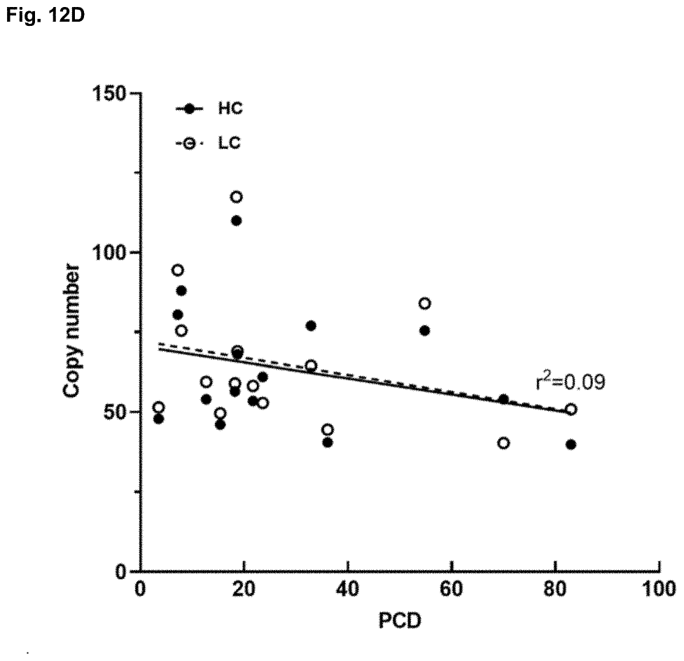

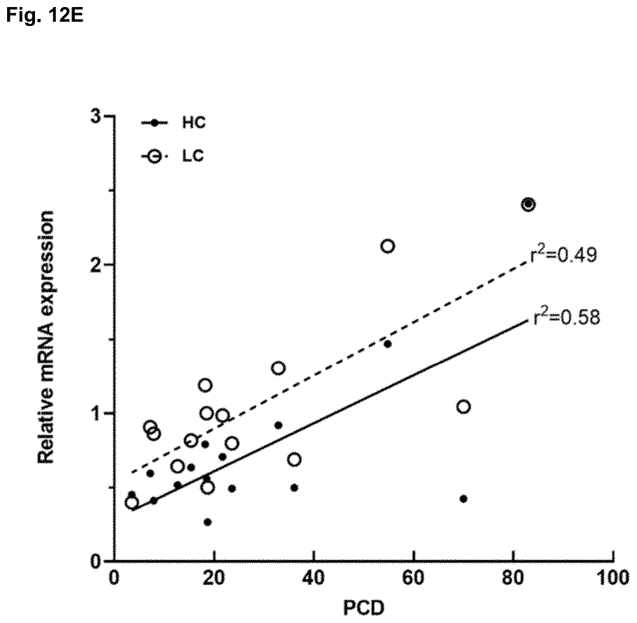

Bicistronic vectors expressing the mAbs transtuzumab and 9-24 had the ORF of either one LC or one HC driven by a CMV promote placed upstream of an IRES driving the ORF of one of the 8 rescue enzymes (FIG. 12A). For 9-24, we constructed an extra set of tricistronic vectors for comparison. The tricistronic vectors had an arrangement analogous to the bicistronic counterparts except the ORFs of LC and HC were both included in each vector with the LC driven by the CMV promoter and the HC driven by an additional IRES. This arrangement has been designed to yield a ratio of LC to HC peptide expression of approximately 4 to 1 (Ho et al. 2012). All of the vectors had one ITR sequence placed upstream of the CMV promoter and one downstream of the SV40 pA sequence; these are the sites used by Sleeping Beauty transposase (SB100X) for integration. To construct the bicistronic vectors we first cloned the ORF of the light chain of trastuzumab into the NsiI site of the rescue vectors described above bearing either Dhodh, Umps, Paics or Atic and cloned the ORF of the heavy chain into the NsiI site of the recue vectors bearing Ctps1, Tyms, Impdh2 or Gmps to create vectors referred to as pre-expression vectors. The primers used to amplify the ORFs of light chain and heavy chain from the vectors used in our previous report (Zhang et al. 2020) are provided in Table 12. The amplified ORFs had tails overlapping the two ends of the Nsi-digested rescue vectors. Ligation was performed using NEBuilder® HiFi DNA Assembly Master Mix to create the 8 pre-expression vectors. The pre-expression vectors were used as precursors to construct the expression vectors used for transposase-aided transfection. The cargo fragment added to each pre-expression vector included the CMV promoter, the ORF of the light chain or heavy chain, the internal ribosome entry site (IRES), the ORF of the rescue enzyme and the SV40 signal. These arrays of fragments were amplified by PCR (primer sequences are provided in Table 12). The amplified products were then cloned into the PflmI and SphI sites of the vector pSBbi-Bla (Addgene), replacing its longer fragment. The resulting plasmid contains the array of expression sequences flanked by two inverted terminal repeat (ITR) sequences recognized by the transposase Sleeping Beauty 100X (SB100X). Each newly created expression vector had a trastuzumab light chain or heavy chain and one of the rescue enzymes placed after a weak IRES, and the entire array was flanked by ITRs.

TABLE 12
Primers for the construction of expression
vectors of trastuzumab.

| | Modules | |
|---|---|---|
| Vector | amplified | Primers |
| Pre- | ORF of | Forward: |
| expression | light | TCCCAGGTCCAACTGCA |
| vectors | chain | GGTCGAGCGCCGCCACC |
| | | ATGTCCGTG |
| | | (SEQ ID NO: 74) |
| | | Reverse: |
| | | GAGGGGCGGAATTGGCC |
| | | GCCCTAGATGCATCAGC |
| | | ACTCGCCCCGGTTG |
| | | (SEQ ID NO: 75) |
| | ORF of | Forward: |
| | heavy | TCCCAGGTCCAACTGCA |
| | chain | GGTCGAGCGCCGCCACC |
| | | ATGGAATGGT |
| | | (SEQ ID NO: 76) |
| | | Reverse: |
| | | AGAGGGGCGGAATTGGC |
| | | CGCCCTAGATGCATCAC |
| | | TTGCCGGGGCTCAGA |
| | | (SEQ ID NO: 77) |
| Expression | CMV, | Forward: |
| vectors | ORF | AGCCATAGAGCCCACC |
| | of light | GCATCCCCAGCATGCG |
| | or | ATGTACGGGCCAGATA |
| | heavy | TA |
| | chain, | (SEQ ID NO: 78) |
| | IRES, | Reverse: |
| | ORF | CCCGAGTAGCTAGTTCA |
| | of | TGGCAGCCAGCATGTCG |
| | rescue | ACGGTATACAGACATG |
| | enzyme | (SEQ ID NO: 79) |
| | and SV40 | |

To construct the biscistronic expression vectors of 9-24, each trastuzumab expression vector was cut by AgeI and NsiI and the large fragment was purified for ligation with the ORF of the light chain or heavy chain of 9-24 (the ORFs with overlapping flanking sequences synthesized by IDT) using NEBuilder® HiFi DNA Assembly Master Mix.

To create tricistronic vectors of 9-24, we first ligated the ORF of the 9-24 light chain, a fragment of IRESwt (Ho et al. 2012) and the ORF of the 9-24 heavy chain using NEBuilder® HiFi DNA Assembly Master Mix. The product was then cloned between the AgeI and NsiI sites in one of the expression vectors (ATIC) of trastuzumab. The resulting tricistronic vector was cut by AgeI and NsiI to supply the ORF of the light chain, the IRESwt and the ORF of heavy chain for cloning into the same sites in the other 7 expression vectors of trastuzumab, creating a total of 8 tricistronic vectors of 9-24.

We transfected the expression vectors (0.5 μg of each for a total of 4 μg) with 0.2 μg of the SB100X transposase-coding vector (pCMV(CAT)T7-SB100, Addgene) into CHO8A or CHO8A-S (see below) cells seeded at 5×10⁵ per well in 6-well plates the day before transfection. The expression vectors for trastuzumab were also transfected into CHO8A cells without SB100X. Two days later, the cells were transferred to 100 mm dishes and incubated in selective medium for 10-12 days. Solitary colonies were then isolated and expanded for determination of trastuzumab or 9-24 secreted in the medium. For measurement of specific productivity (pg/cell/day, pcd), pooled colonies or single clones were seeded at 1×10⁶ in 6-well plates and the medium was collected after incubation for 24 h. The concentration of trastuzumab or 9-24 was determined by enzyme-linked immunosorbent assay (ELISA). The highest producer of trastuzumab was passaged in selective medium for 3 months. After each indicated number of weeks a portion of the cells was collected and used for determination of productivity.

Determination by ELISA of Trastuzumab and 9-24 Secreted into the Medium

To perform the ELISA assay, 96-well plates were coated with capture antibody (AffiniPure Goat Anti-Human IgG (H+L), from Jackson Labs, using 100 μl of a 1:500 dilution in 0.05 M carbonate buffer (pH 9.6). After overnight incubation at 4° C., the plate was washed three times with TBST (50 mM Tris buffered saline with 0.05% TWEEN® 20) followed by the addition of 100 μl of diluted medium samples from trastuzumab or 9-24 expressing CHO8A cells or standards and incubation for 2 h at room temperature. After three washes with TBST buffer, 100 μl of secondary antibody, goat anti-Human IgG Fc cross adsorbed, HRP (ThermoFisher Scientific; cat. no. PI31413) at a 1:2000 dilution in TBS with 1% BSA was added and the plate was incubated for 1 h at room temperature and then washed three times with TBST. ABTS substrate solution (100 μl, ThermoFisher Scientific; cat. no. PI37615) was added to each well and developed at room temperature for 10 min. The reaction was stopped by adding 100 μl of 1% SDS to each well. The absorbance was recorded on a plate reader at a wavelength of 405 nm.

### Determination of Copy Numbers of Transgenes

The QX200 Droplet Digital PCR System (Bio-Rad, Hercules, Calif.) was used to determine the copy number of LC, HC and all of the rescue enzymes in cell clones of trastuzumab. Manufacturer recommended protocols were followed for the extraction of genomic DNA using the DNeasy Blood and Tissue Kit (QIAGEN, Germany). Approximately 25 ng of DNA was used for each ddPCR reaction, using FAM-labeled fluorescent probes (sequences provided in Table 13) designed by the Primer Express Software (Applied Biosystems, Thermo Fisher Scientific, MA), and synthesized by Invitrogen (Thermo Fisher Scientific, MA).

TABLE 13
Primers, probes for copy
number measurement by ddPCR.

| Gene | Name | sequence |
|---|---|---|
| Atic | Atic | CCTTGCAGGTGATAAG |
| | fwd | GCAAA |
| | | (SEQ ID NO: 80) |
| | Atic | GGAAAGCACTCGTGGA |
| | rev | TGGT |
| | | (SEQ ID NO: 81) |
| | Atic | TCGTGGTGGCTGCG |
| | probe_FAM | (SEQ ID NO: 82) |
| Ctps1 | Ctps1 | CCATGTGGAACCTGAAC |
| | fwd | AAGTG |
| | | (SEQ ID NO: 83) |
| | Ctps1 | CAAGGGTACCCGGTAGA |
| | rev | TGGA |
| | | (SEQ ID NO: 84) |
| | Ctps1 | TCTGTGTTCATGATGTT |
| | probe_FAM | TC |
| | | (SEQ ID NO: 85) |
| Dhodh | Dhodh | ACGGCCCAGGACAAGGA |
| | fwd | (SEQ ID NO: 86) |
| | Dhodh | CCATCAATGCCCAGCTC |
| | rev | TCT |
| | | (SEQ ID NO: 87) |
| | Dhodh | ACATTGCCAGTGTGGC |
| | probe_FAM | (SEQ ID NO: 88) |
| Gmps | Gmps | GCAAAGTCATCGACCGA |
| | fwd | AGAG |
| | | (SEQ ID NO: 89) |
| | Gmps | TATGGCGAAGGCTGGTG |
| | rev | TTT |
| | | (SEQ ID NO: 90) |
| | Gmps | CGGGAGCTCTTTGTGCA |
| | probe_FAM | (SEQ ID NO: 91) |
| Impdh2 | Impdh2 | GGCTTCATCCACCACAA |
| | fwd | CTGT |
| | | (SEQ ID NO: 92) |
| | Impdh2 | TGAATCCCTGTTCATAT |
| | rev | TTCTTTACTTTC |
| | | (SEQ ID NO: 93) |
| | Impdh2 | CCAGGCCAATGAAGT |
| | probe_FAM | (SEQ ID NO: 94) |
| Paics | Paics | AAGGCGATGGCATACCT |
| | fwd | ACTGT |
| | | (SEQ ID NO: 95) |
| | Paics | GGCCCAAACCATTGCTT |
| | rev | CT |
| | | (SEQ ID NO: 96) |
| | Paics | TTGTCGCAGTGGCTGG |
| | probe_FAM | (SEQ ID NO: 97) |
| Tyms | Tyms | CGGTGTTCGGCATGCA |
| | fwd | (SEQ ID NO: 98) |
| | Tyms | GGTTGTGAGCAGAGGAA |
| | rev | ATTCAT |
| | | (SEQ ID NO: 99) |
| | Tyms | CGCGATACAGCCTGAGA |
| | probe_FAM | (SEQ ID NO: 100) |
| Umps | Umps | CTGGTCACCGAGCTGTA |
| | fwd | CGA |
| | | (SEQ ID NO: 101) |
| | Umps | GGTGAGCCCGCTCTTCA |
| | rev | G |
| | | (SEQ ID NO: 102) |
| | Umps | CAGGCTTTCAAGTTC |
| | probe_FAM | (SEQ ID NO: 103) |

The total transgene copy numbers of the pooled CHO8A transfected with 1, 2 or 8 tricistronic vectors of 9-24 were determined by Real Time PCR (qPCR). The genomic DNA from the pool was prepared using Quick-DNA™ Miniprep Kit (Zymo Research) according to the manufacturer's instructions. DNA (12.5 ng) was subjected to Real time PCR containing 250 nM primers and Luna® Universal qPCR Master Mix (NEB) by the StepOnePlus™ Real-Time PCR System (Applied Biosystems). The PCR conditions were as follows: 95° C. for 1 min; 95° C. for 15 s, 60° C. for 30 s for 40 cycles in a total volume of 10 μL. Two pairs of primers (Kolacsek et al. 2011) were designed to target the left ITR (ITR_L) and right ITR (ITR_R) of the vectors, respectively. The gene Tpgs2 with a reported copy number of one in CHO-K1 cells (the ancestor of CHO8A) (Kaas et al. 2015) served as a reference gene. The sequences of primers are provided in Table 14. The PCR efficiency for the primers of ITR_L, ITR_R and Tpgs2 were determined as 105%, 98% and 104%, respectively. The total copy number of transgenes was calculated based on 2^{-delta CT} where delta C_{T}=(C_{T} ITR_L or ITR_R−C_{T} Tpgs2).

TABLE 14
The primers used in the Real-Time PCR.

| | | sequence |
|---|---|---|
| ITR_L | Forward | CTCGTTTTTCAACTAC |
| | | TCCACAAATTTCT |
| | | (SEQ ID NO: 110) |
| | Reverse | GTGTCATGCACAAAGT |
| | | AGATGTCCTA |
| | | (SEQ ID NO: 111) |
| ITR_R | Forward | GCTGAAATGAATCATT |
| | | CTCTCTACTATTATTC |
| | | TGA |
| | | (SEQ ID NO: 112) |
| | Reverse | AATTCCCTGTCTTAGG |
| | | TCAGTTAGGA |
| | | (SEQ ID NO: 113) |
| Tpgs2 | Forward | TGGATAAAATTGCTTC |
| | | ATTCCCTGA |
| | | (SEQ ID NO: 114) |
| | Reverse | AAGATATTGACTCTAA |
| | | CGGAGGAAC |
| | | (SEQ ID NO: 115) |

Determination of mRNA Expression by NanoString

Pellets of cloned cells or a CHO K1-based laboratory cell line, GSKO-aHer2, were lysed in Buffer RLT (Qiagen, diluted 1:3 in distilled water) by vortexing. Lysates were then cleared by spinning at 2,000 g for 2 minutes, and the supernatant was used directly in the assay. In brief, the supernatants were mixed with nCounter PlexSet™ reagents (NanoString Technologies, Seattle, Wash.) and a custom probe library (Table 15) in a 96-well plate and hybridized for 16 hours at 67° C., then cooled to room temperature. Hybridized samples were then processed and transferred to an nCounter cartridge on the nCounter Prep Station according to manufacturer's instructions. The loaded cartridge was then analyzed on the nCounter Digital Analyzer using maximum resolution settings. The resulting raw data was processed and normalized to internal housekeeping genes (Table 15) using nSolver 4 software.

TABLE 15
The probes for individual rescue enzymes,
LC, HC and housekeeping genes
used in NanoString.

| | Gene | Target Sequence |
|---|---|---|
| | Umps | GATCATTGAG |
| | | GATGTCGTCA |
| | | CCAGTGGGGC |
| | | CAGTGTTTTG |
| | | GAAACTGTTG |
| | | AAGTTCTTCA |
| | | GAAGGAGGGC |
| | | CTGAAGGTGA |
| | | CTGATGCCAT |
| | | AGTGCTGTTA |
| | | (SEQ ID |
| | | NO: 116) |
| | Atic | GGCGCCACTT |
| | | GGCTTTGAAG |
| | | GCATTTACTC |
| | | ATACTGCACA |
| | | ATATGATGAA |
| | | GCGATTTCAG |
| | | ATTACTTCAG |
| | | AAAGCAGTAC |
| | | AGTAAGGGGA |
| | | TTTCCCAGAT |
| | | (SEQ ID |
| | | NO: 117) |
| | Dhodh | CATCCTTGGG |
| | | GGAGGAGGAC |
| | | TTCTCTTCAC |
| | | CTCTTACCTG |
| | | ACAGCCACGG |
| | | GCGATGACCA |
| | | TTTCTATGCT |
| | | GAATATCTGA |
| | | TGCCGGCCCT |
| | | GCAGAGGCTG |
| | | (SEQ ID |
| | | NO: 118) |
| | Ctps1 | AGTGGTAGAA |
| | | TTCTCAAGAA |
| | | ATGTGCTGGG |
| | | ATGGCAAGAT |
| | | GCCAATTCTA |
| | | CAGAATTTGA |
| | | CCCTAAGACT |
| | | AGTCATCCTG |
| | | TGGTCATAGA |
| | | CATGCCAGAA |
| | | (SEQ ID |
| | | NO: 119) |
| | Tyms | TGTCGGTGTT |
| | | CGGCATGCAG |
| | | GCGCGATACA |
| | | GCCTGAGAGA |
| | | TGAATTTCCT |
| | | CTGCTCACAA |
| | | CCAAAAGAGT |
| | | GTTCTGGAAG |
| | | GGAGTTTTGG |
| | | AGGAGTTGCT |
| | | (SEQ ID |
| | | NO: 120) |
| | Paics | TAAAGCAGAG |
| | | TATGAAGGCG |
| | | ATGGCATACC |
| | | TACTGTATTT |
| | | GTCGCAGTGG |
| | | CTGGCAGAAG |
| | | CAATGGTTTG |
| | | GGC |
| | | CCAGTGATGT |
| | | CTGGTAATAC |
| | | TGCATAT |
| | | (SEQ ID |
| | | NO: 121) |
| | Gmps | CGAACCTTCA |
| | | TTACTAGTGA |
| | | CTTCATGACT |
| | | GGTATAGCTG |
| | | CAACACCTGG |
| | | CAATGAGATA |
| | | CCTGTAGAGG |
| | | TGGTACTAAA |
| | | GATGGTCACT |
| | | GAGATAAAGA |
| | | (SEQ ID |
| | | NO: 122) |
| | Impdh2 | CCCAAGCAAC |
| | | AGCAGTGTAC |
| | | AAGGTTTCTG |
| | | AGTATGCTCG |
| | | GCGCTTTGGT |
| | | GTTCCTGTTA |
| | | TTGCTGATGG |
| | | AGGAATCCAA |
| | | AATGTGGGTC |
| | | ATATTGCCAA |
| | | (SEQ ID |
| | | NO: 123) |
| | HC | CAGCTGAAGT |
| | (trastuzumab) | CCGGCACAGC |
| | | TTCTGTCGTG |
| | | TGCCTGCTGA |
| | | ACAACTTCTA |
| | | CCCTCGGGAA |
| | | GCCAAGGTGC |
| | | AGTGGAAGGT |
| | | GGACAATGCC |
| | | CTGCAGTCCG |
| | | (SEQ ID |
| | | NO: 124) |
| | LC | AAGACCAAGC |
| | (trastuzumab) | CTAGAGAGGA |
| | | ACAGTACAAC |
| | | TCCACCTACA |
| | | GAGTGGTGTC |
| | | CGTGCTGACC |
| | | GTGCTGCACC |
| | | AGGATTGGCT |
| | | GAACGGCAAA |
| | | GAGTACAAGT |
| | | (SEQ ID |
| | | NO: 125) |
| House | Yaf2 | AGGAAGCAAC |
| keeping | | TAGCAAAAAG |
| genes | | AACTGTCATA |
| | | AAAAAACCAG |
| | | ACCAAGATTA |
| | | AAAAATGTGG |
| | | ATCGGAGTAG |
| | | CGCTCAGCAT |
| | | TTGGAAGTCA |
| | | CTGTTGGAGA |
| | | (SEQ ID |
| | | NO: 126) |
| | Ap3d1 | AGTTGAGGAT |
| | | ATTGATAGAA |
| | | GACTCAGACC |
| | | AGAACTTAAA |
| | | GTACCTGGGG |
| | | CTTCTGGCCA |
| | | TGTCTAAAAT |
| | | CCTGAAGACA |
| | | CACCCCAAGT |
| | | CCGTGCAGTC |
| | | (SEQ ID |
| | | NO: 127) |
| | Copa | GCTGCATTAT |
| | | GTAAAGGACC |
| | | GATTCCTCCG |
| | | TCAGCTGGAT |
| | | TTCAACAGCT |
| | | CCAAAGATGT |
| | | AGCTGTGATG |
| | | CAGTTGAGGA |
| | | GTGGTTCCAA |
| | | GTTTCCAGTG |
| | | (SEQ ID |
| | | NO: 128) |
| | Mmadhc | AACAGATGAA |
| | | CGCTACCGGC |
| | | ATTTAGGATT |
| | | CTCTGTTGAT |
| | | GACCTTGGCT |
| | | GCTGTAAAGT |
| | | GATCCGTCAT |
| | | AGTCTCTGGG |
| | | GTACTCATGT |
| | | GTTTGTAGGA |
| | | (SEQ ID |
| | | NO: 129) |
| | Tmed2 | TGTCCGTTGT |
| | | TGGCACTTAG |
| | | GAAGAGCGAA |
| | | ATTGCTCAAG |
| | | TGGAAGCCTG |
| | | CATTTCTCAT |
| | | GCCAAAGGGT |
| | | TAAGCCTCTT |
| | | GGATGGCTTG |
| | | CAATGATAGG |
| | | (SEQ ID |
| | | NO: 130) |
| | Pabpn1 | ATGCCCGTTC |
| | | TATCTACGTT |
| | | GGCAATGTGG |
| | | ACTATGGTGC |
| | | AACAGCAGAA |
| | | GAGCTGGAAG |
| | | CTCACTTTCA |
| | | TGGTTGTGGT |
| | | TCAGTCAACC |
| | | GTGTCACTAT |
| | | (SEQ ID |
| | | NO: 131) |
| | Gnb1 | GTGGACACCC |
| | | ACACCTCCCC |
| | | TCAGAACTTC |
| | | AAAAGGGCAA |
| | | CATCTTTTTT |
| | | TCCTTCACTT |
| | | ATTGCTGAAA |
| | | CCAAGAGCAC |
| | | AACTCCCATT |
| | | CAGAGAAGGA |
| | | (SEQ ID |
| | | NO: 132) |
| | Actr5 | GGTTGCCTAT |
| | | GGGATAGATA |
| | | GTCTCTTCAG |
| | | CTTCTATCAC |
| | | AACATGCCCA |
| | | AGAATGCCCT |
| | | TTCCAGCGGC |
| | | CTCATCATTT |
| | | CATCCGGCTA |
| | | CCAGTGTACA |
| | | (SEQ ID |
| | | NO: 133) |

### Adaptation of CHO8A and Two High Producers to Protein-Free CD CHO Medium

The CHO8A cells and two isolated cell clones producing high levels of trastuzumab (TraSB-6) and 9-24 (9-24SB-5) that had been cultured as adherent cells in serum-supplemented medium were inoculated at 1×10⁶ cells/ml into 125 ml flasks (Optimum Growth™ Flasks, Thomson Instrument Company) with 30 ml of protein-free CD CHO medium. In the case of CHO8A, the medium was supplemented with uridine, thymidine, cytidine, adenine and guanine, each at 100 μM. L-glutamine at 4 mM, Anti-clumping agent A (Lonza) at 1:100 v/v, 100 U/ml penicillin and 100 μg/ml streptomycin were also added to the CD CHO medium. The flasks were placed on an orbital shaker (orbital radius: ¾″) at 125 rpm in a 5% CO₂ and 95% humidified incubator at 37° C. During 20-24 days of adaptation, CHO8A and TraSB-6 were collected every 3-5 days by centrifugation and re-suspended in fresh medium. For transfection, adapted CHO8A-S cells were returned to serum-containing medium and cultured as adherent cells for the transfection of the 8 expression vectors for 9-24. Following cloning, 9-24SB-5 were capable of re-adapation to the CD CHO medium within 1 week of culture. At the end of adaptation, the adapted cells were expanded under the same conditions as those for the adaptation. The growth curves for each cell lines and doubling time were then determined for 11 days of culture. At the same time, the medium supernatant was collected for determination of the concentrations of trastuzumab and 9-24 by ELISA. The specific productivity (Qp) was calculated according to the equation: Qp=Titer/IVCC (Integral Viable Cell Concentration).

### Results and Discussion

### Knockout of 8 Steps in Pyrimidine and Purine Biosynthesis Pathways in CHO Cells

We have previously reported the CRISPR-Cas9-induced knockout of the pyrimidine pathway enzyme gene Umps and the purine pathway enzyme gene Atic in CHO-K1 cells giving rise to the double auxotroph cell line UA10. UA10 cells can grow if provided with a source of pyrimidines and purines or if transfected with cDNA-based versions of these 2 genes (Zhang et al. 2020). We reasoned that a cell line with additional enzyme deficiencies would demand higher order co-transfections and hence enhance productivity of a protein of interest by guaranteeing an increased copy number of integrated cargo genes. Toward this end we used CRISPR-Cas9 mutagenesis to similarly knock out 6 additional enzymatic steps in the pyrimidine/purine pathways, creating the cell line CHO8A. To grow in a medium devoid of purines and pyrimidines CHO8A requires the incorporation of 8 rescue genes coding for the 8 missing enzymatic activities. The genes and steps knocked out in CHO8A are shown in FIG. 6. The targeted steps for CTPS and IMPDH can be catalyzed by two pairs of isozymes, CTPS1/CTPS2 and IMPDH1/IMPDH2, respectively, requiring that a total of 10 genes had to be knocked out to create the 8 deficiencies: Dhodh, Umps, Ctps1, Ctps2, and Tyms in the pyrimidine pathway and Paics, Atic, Impdh1, Impdh2 and Gmps in the purine pathway. To minimize the frequency of reversion and to assure enzyme non-expression, all of the mutants chosen involved insertions or deletions that caused a frameshift. A description of the mutations carried by CHO8A and the stepwise isolation of intermediate cell lines are shown in Table 6.

CHO8A cells grew well, with a 16.6 hour doubling time compared to 16.2 h for CHO-K1, if provided with a combination of 5 nutrients (uridine (U), cytidine (C), thymidine (T), guanine (G) and either hypoxanthine (H) or adenine (A) that are assimilated via salvage pathways to circumvent the deficiencies (FIG. 6). Specifically, U, C, and T circumvent the deficiencies in DHODH plus UMPS, CTPS1/2 and TYMS, respectively. A or H circumvents the deficiencies in PAICS and ATIC; and G circumvents the deficiencies in IMPDH1/2 and GMPS.

### Rescued Expression of the Enzymes Deficient in CHO8A Cells

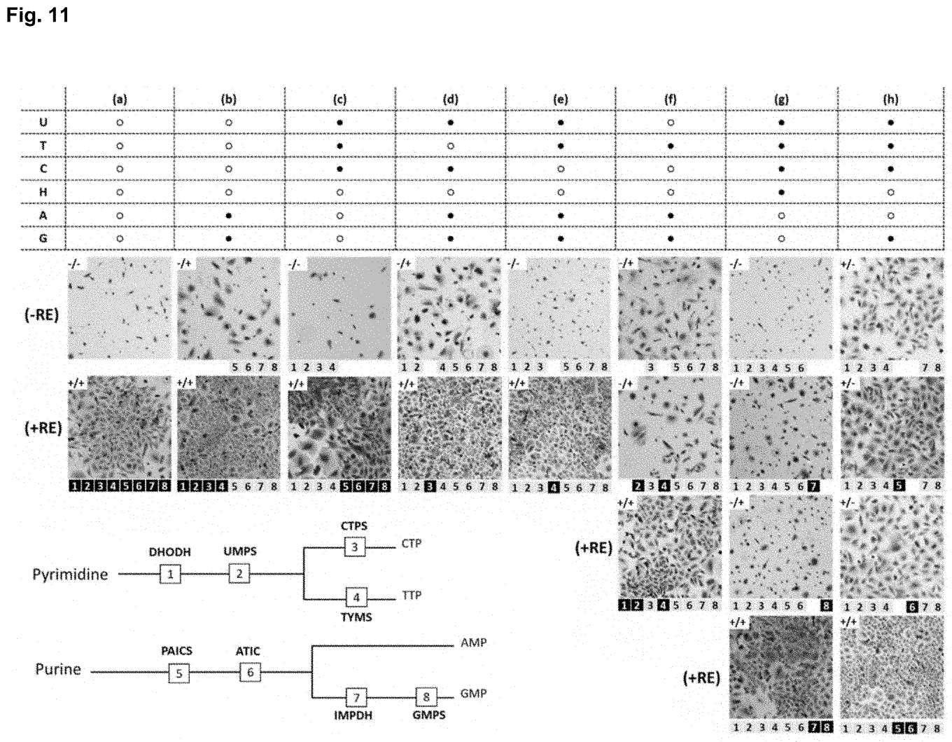

Growth of CHO8A cells in the absence of purine/pyrimidine nucleotide precursors can be achieved by simultaneous permanent transfection with 8 plasmid rescue vectors that together provide genes coding for the 8 missing enzyme activities (FIG. 11, first column, +RE). The rescue genes here feature an enzyme ORF driven by a CMV promoter, an intron in the 5′ UTR and are terminated with an SV40 polyA site. The expression cassettes are flanked by Sleeping Beauty inverted terminal repeat sequences (ITRs). Although there are 2 endogenous genes each for CTPS and IMPD in the CHO genome each of which codes for a distinct isoform, we found that provision of a single isoform (CTPS1 and IMPDH2, respectively) was sufficient for growth. Growth of CHO8A was also assessed by combining particular supplementations with corresponding transfections using specific rescue vectors. Such combinations were used to verify specific deficiencies in CHO8A as well as the capability of rescue vectors to compensate for the deficiencies. Eight groups (a-h) of transfections were categorized based on the enzyme gene(s) used for rescue and by manipulating the nutrients in the medium. In a medium devoid of pyrimidines and purines, transfection with all 8 rescue vectors (UMPS, ATIC, DHODH, CTPS1, TYMS, PAICS, IMPDH2 and GMPS) enabled growth (FIG. 11, group a). In a medium devoid of pyrimidines (U, T, C) but supplemented with purines (A, G), exogenous DHODH, UMPS, TYMS and CTPS1 genes were necessary and sufficient for growth (FIG. 11, group b), while in a medium supplemented only with pyrimidines (U, T, C) CHO8A required exogenous PAICS, ATIC, IMPDH2 and GMPS genes (group c). We previously showed that the UA10 cell line, the precursor of CHO8A that is deficient only in UMPS and ATIC, could be rescued by the expression of corresponding exogenous rescue genes (Zhang et al. 2020). Here we demonstrated that the other 6 enzymatic steps knocked out in CHO8A cells: TYMS (group d), CTPS (group e), DHODH (group f), IMPDH plus GMPS (group g) and PAICS (group h) could also be rescued by exogenous expression of the corresponding rescue enzymes. For combinations a through g in FIG. 11 some cell attachment but no growth or sparse growth was seen unless the cells were transfected with the appropriate rescue vector(s). For combination (h), designed to require ATIC and PAICS genes, a background of cell growth with loosely packed cells was evident when either the ATIC or PAICS gene alone was provided. Colonies of healthy cells could be readily differentiated amidst this background by rescued expression of both enzymes, as shown in the bottom photo of the last column of FIG. 11. This background might be attributable to the guanine present in this medium; guanine can be salvaged to GMP by hypoxanthine-guanine phosphoribosyltransferase and thence to IMP by GMP reductase (Deng et al. 2002) albeit apparently to a marginal extent.

### Flexibility of CHO8A Cells for the Production of Recombinant Proteins

The multiple deficiencies and combinatorial rescues of CHO8A cells provide a flexible platform for the expression of recombinant protein(s). The requirement of 8 rescue enzymes can be translated into 8 selective markers for permanent incorporation of multiple transgenes. Eight transgenes each bearing one of 8 rescue enzymes and a particular cargo gene could be simultaneously transfected into CHO8A cells followed by selection for all in one fell swoop, and without the need of any antibiotics or toxic chemicals. Alternatively, by the judicious choice of the nutrients in the medium, different numbers (1, 2, 3, 4, 5, 6, 7 or 8, shown in Table 7) of transgenes can be transfected into CHO8A cells and readily selected, examples of which are shown in FIG. 2. For example, if hypoxanthine and uridine are supplemented it would leave 4 downstream enzymatic steps (TYMS, CTPS, IMPDH and GMPS) for rescue, allowing simultaneous selection for the guaranteed permanent integration of 4 transgenes. It should be noted that in its simplest mode, only a single rescue vector need be constructed and used (e.g., columns d and e in FIG. 11).

Another way to exploit the properties of CHO8A cells would be to use serial rather than simultaneous transfections. As many as 7 serial transfections could be designed if two additional nutrients, orotic acid (O) and 5-aminoimidazole-4-carboxamide-1-β-D-ribofuranoside (AICAR) shown in FIG. 1, were to be employed to circumvent the deficiencies in DHODH and PAICS, respectively (Table 9). The serial introduction of same transgene of interest in all 8 vectors would be expected to yield a multi-fold amplification in transgene copy number compared to a single transfection. A much faster alterative for such multi-fold amplification would be the simultaneous transfection of all 8 rescue vector carrying the same cargo, which we carried out as described below.

TABLE 9
Scheme for 7 serial transfections in CHO8A cells.

| Transfection No. | Rescue transgene | Supplements |
|---|---|---|
| 1 | TYMS | U, C, A, G |
| 2 | CTPS1 | U, A, G |
| 3 | UMPS | O^{a}, A, G |
| 4 | DHODH | A, G |
| 5 | GMPS + IMPDH2 | H |
| 6 | ATIC | AICAR^{b} |
| 7 | PAICS | none |

^{a}orotic acid;
^{b}5-aminoimidazole-4-carboxamide ribotide

### Production of Trastuzumab and Anti-SARS-Cov-2 Antibody 9-24 in CHO8A Cells

One useful application of CHO8A cells would be the simultaneous or serial incorporation of 8 plasmids carrying different cargo genes to create a cell line expressing 8 different polypeptides, a feature not addressed here. What is addressed here is a second use: increasing the production of single proteins by increasing the gene copy numbers and the genomic distribution of their constituent polypeptides. Namely, we tested the ability of CHO8A cells to produce 2 mAbs: trastuzumab and an anti-SARS-Cov-2 antibody, 9-24, kindly provided by David Ho's laboratory. We chose to use transgenes delivered as Sleeping Beauty (SB100X) transposons since they generate a wide range of both expression levels (Querques et al., 2019) and transgene copy numbers (Grabundzija et al, 2010). This wide distribution and near random integrations of transposon transgenes fits well with the selective pressure applied to incorporate all 8 of distinct vectors in the applied mixture. That is, the selected survivors should be sampling the high end of these distributions. We first tested this idea by comparing the average copy number of transgenes in selected pools of CHO8A cells transfected with 1, 2 or 8 rescue vectors. In this experiment we used tricistronic transposon vectors, each carrying both the LC and HC genes of mAb 9-24 in addition to the rescue gene (see Methods). Transfection with 1 or 2 rescue vectors yielded 28 and 35 vector copies per cell, respectively, while the simultaneous use of all 8 rescue vectors indeed raised this number to 90.

We next tested the proficiency of CHO8A cells to express and secrete the mAbs trastuzumab or 9-24. For these experiments we opted to use bicistronic vectors (FIG. 3a), each carrying either a LC or a HC gene along with one of the selective markers. Uncoupling these 2 genes allows the generation by chance of transfectants carrying varying ratios of each. In this way we would have the opportunity to simultaneously screen for an optimum LC:HC expression ratio as well as a high individual expression level of each gene. Transfection of 3 million cells for production of trastuzumab without the use of transposase yielded only 5 colonies in the selective medium compared to a typical result of many hundreds when selecting for only a single marker. Using the transgenes as transposons, i.e., by co-transfection with the SB100X transposase gene, was much more efficient, yielding thousands of colonies. The latter were collected as a pool for measurement of the secretion of trastuzumab; a specific productivity (Qp) of 20 pcd (picograms per cell per day) was obtained. We previously reported that the use of 2 of these markers (UMPS and ATIC) with the UA10 cell line, the progenitor of CHO8A, yielded a trastuzumab Qp of 5.4 pcd in pooled permanent transfectants. Thus the use of transposons and increasing the number of selective markers from 2 to 8 produced a near 4-fold increase in productivity. We also measured the secretion of trastuzumab in 10 clones isolated from the pool produced with transposase (TrasSB) and in all 5 of the clones generated without the enzyme (Tras). The average Qp was 35 pcd with transposase and 16 pcd without it (FIG. 12B). Aside from this 2-fold enhancement of average Qps, the use of transposons here showed a second important difference: the distribution of Qps among transfectant clones was much wider when transposase was used, exhibiting a 20-fold range among the 10 clones examined compared to a 3-fold range without transposase (FIG. 12B). The wide range with transposase yielded a top clone with a Qp of 83.6 pcd, 4 times greater than the highest clone without transposase. Thus the use of transposition and CHO8A multiple selections provides an effective means for the isolation of high producing clones using minimal screening. This highest producer, TrasSB-6, was expanded for further use. TrasSB-6 sustained its productivity during 3 months of continuous culture in standard purine/pyrimidine-free selective medium (FIG. 12C). To check whether these results were peculiar to trastuzumab, we repeated the SB100X transfection of mAb 9-24 genes with 8 rescue vectors carrying the LC or the HC. The average Qp of pooled survivors for 9-24 was 41 pcd. Ten randomly chosen transfectant cell clones once again displayed a wide range of Qp values with the highest (9-24SB-5) reaching 137 pcd, as shown in FIG. 12B.

### Gene Copy Numbers

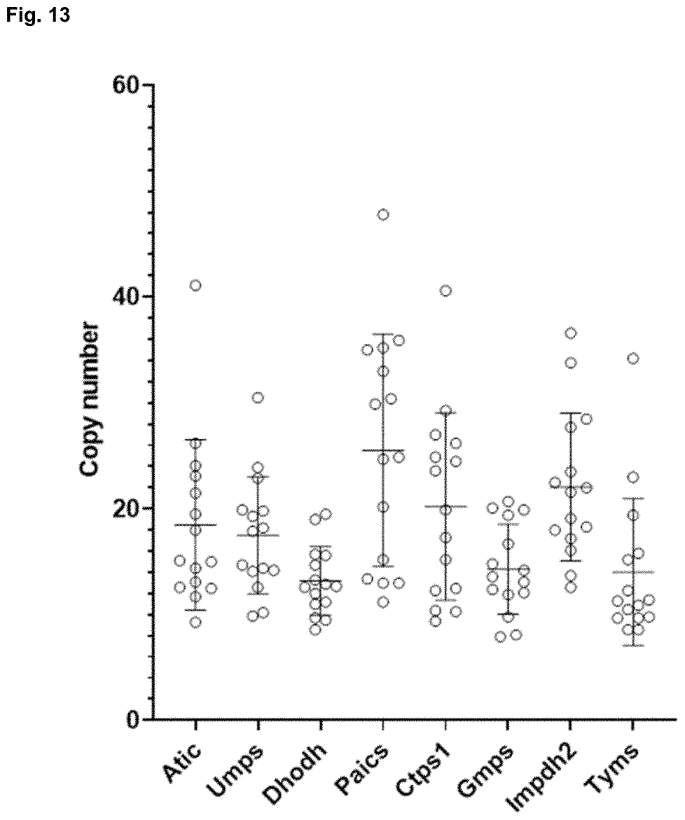

A principal motivation for the isolation of CHO8A cells was to test the idea that selection for the integration of multiple distinct rescue genes would favor the isolation of transfectants carrying higher copy numbers of cargo transgenes. Measurements of LC and HC gene copy numbers by droplet digital PCR (ddPCR) showed that this was indeed the case: LC and HC copy numbers among the 15 Tras plus TrasSB clones ranged from 40 to 117 per cell (average of 64), as can be seen in FIG. 12D, which also shows that the LC and HC gene copy numbers were closely matched in individual clones. The high end of this range is substantially greater than the 28 copies typically found in single marker selection experiments and the average of 18 copies seen for individual rescue genes (FIG. 13). However, a quantitative relationship between copy number and productivity was not seen (FIG. 12D), suggesting that beyond about 40 copies per cell, other factors are limiting gene expression. This non-concordance is exemplified by clone TrasSB-6, which is the highest trastuzumab producer yet has a somewhat below average copy number of 51 LC and 40 HC genes. Interestingly, copy numbers were not different with or without the use of transposase (an average of 68 vs. 63 copies per cell for LC and of 62 vs. 64 for HC, respectively), but was as expected if this bias is mainly driven by the requirement for incorporation of all 8 rescue enzymes.

Transgene mRNA Expression

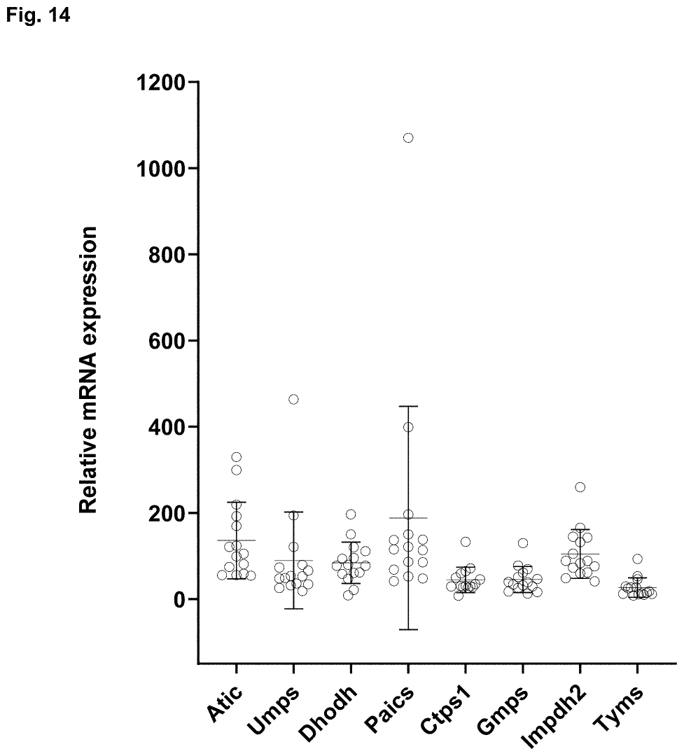

We used NanoString to measured mRNA levels of the LC and HC genes in the isolated cell clones and those of the individual rescue enzyme genes as well. For a comparison, the mRNA levels (molecules per cell) were normalized to those of a CHO K1 laboratory cell line, GSKO-aHer2, which expresses trastuzumab at a Qp of 35 pcd in a 14-day fed batch culture. TrasSB-6 had the highest trastuzumab mRNA expression (2.4 fold relative to GSKO-aHer2 for both LC and HC, as can be seen in FIG. 12E. TrasSB-1 also had high mRNA expression, LC at 1.5 fold and HC at 2.1 fold with a Qp of 54.8 pcd. In the clones of TrasSB, the mRNA expression of LC and HC correlated positively with Qp (r²=0.48 and 0.57, respectively). The mRNA expression corresponding to the 8 rescue enzymes ranged widely in individual clones, from 8 to 1070 times that of GSKO-aHer2 cells (FIG. 14). The latter should represent wild type CHO K1 expression levels for these endogenous enzyme genes.

### Production of mAb Proteins in Protein-Free Suspension Culture

CHO8A, TrasSB-6 and 9-24SB-5 were adapted to protein-free CD CHO medium and designated as CHO8A-S, TrasSB-6-S and 9-24SB-5-S populations. The Qp value in shake flasks of TrasSB-6-S was 57 pcd in CD CHO, a number usually regarded as a high producer, albeit somewhat lower than the 84 pcd exhibited in serum-containing medium. In the case of mAb 9-24, the adapted CHO8A-S cells were returned to serum-containing medium and cultured as adherent cells for the simultaneous transfection of the 8 transposon vectors carrying the 9-24 LC or HC genes. The highest producer, 9-24SB-5, was then re-adapted to the CD CHO protein-free suspension medium, to which it did quickly, indicating that the adapted cells retained this ability despite their short sojourn and transfection as adherent cells in the presence of serum. 9-24SB-5 exhibited a Qp of 96 pcd in CD CHO medium. CHO8A-S, TrasSB-6-S and 9-24SB-5-S all grew relatively slowly in CD CHO in shake flasks with doubling times of 46 h, 55 h and 54 h, respectively. The undifferentiated slow growth in CD CHO among these 3 suspension cell clines indicates that the relatively slow growth of the two high producers was not due to the burdens of exogenous expression of abundant proteins. Thus for high scale use it remains to either select for CHO8A cells with higher growth rates or to find or develop an optimal protein-free medium for these cells.

In summary, we have applied CHO8A cells to the production of two antibodies: trastuzumab and 9-24. In both cases, highly producing clones were rapidly obtained in single transfections by screening only 10 selected transfectant clones.

### Discussion

We knocked out 10 enzymes catalyzing 8 steps of the pyrimidine/purine biosynthetic pathways in CHO-K1 cells and established a multiply auxotrophic cell line: CHO8A. The growth of CHO8A depends on the presence in the medium of nutrients: uridine, cytidine, thymidine, adenine (or hypoxanthine) and guanine. Alternatively, growth of CHO8A in an ordinary purine/pyrimidine free medium can be realized by exogenous expression of the 8 knocked out enzymes. Such multiple auxotrophs provide an excellent platform for flexible and rapid production of recombinant proteins in CHO cells.

The ability to readily introduce up to 8 different transgenes encoding 8 recombinant polypeptides represents an efficient way to engineer CHO cells to synthesize a variety of polypeptides after a single transfection, since the integration of all 8 transgenes is assured due to selection. Potential cargoes include multiple antibodies, bispecific antibodies, and proteins made up of multiple subunits. The number of 8 need not be a limitation, since 2 or more cargo genes can be easily added to these rescue vectors en masse, as they have identical architectures. Thus multiplex ligations for the expression of more than 16 ORFs in a single experiment can be envisioned. Another ambitious use would be the establishment of CHO cell lines that express tissue-specific mammalian biosynthetic pathways. e.g., neurotransmitters, or multiple nutrient biosynthetic pathways from prokaryotes (Rees & Hay, 1995; Trolle et al. 2021)

We have shown here that these multiple selective markers can also be used for the rapid isolation of transfectant clones that are high producers of single proteins, namely, mAbs. Engineering a requirement for multiple rescue vectors encoding multiple selective markers is a generally applicable strategy to enrich for cells with high copy numbers of integrated transgenes. Although high transgene copy numbers do not guarantee correspondingly high productivities, they are nevertheless a likely prerequisite for such, as can be seen in the data summary presented in Table 10. The expansion of the number of selective markers from 2 in our previous work to 8 in the present work resulted in a several-fold increase in gene copy numbers per cell and an order of magnitude increase in productivity of a winning clone (Table 10). The high copy numbers enabled by the use of transposons coupled with the selection for 8 markers at once has enabled the isolation of high producer clones by screening a mere 10 colonies. One can imagine that the knockout of genes for additional multistep biosynthetic pathways in CHO8A cells, such as that of cholesterol, could allow the isolation of CHO cell lines with even more selective markers. Moreover, this strategy should be portable to cell lines other than CHO.

TABLE 10
Summary of gene copies and expression levels
as a function of selective marker number.

| | No. of | | Productivity (pcd) | | |
|---|---|---|---|---|---|
| | selective | No. of LC + HC | | Range | The |
| | markers | genes in pooled | | among | highest |
| Antibody | used | transfectants | Pool | 10 clones | clone |
| trastuzumab | 2 | ND^{b} | 5.4 | 2.2-6.6 | 6.6 |
| | 8 | 127 | 20 | 3.5-83.0 | 83 |
| 9-24 | 1 | 28^{a} | 0.5 | ND | ND |
| | 2 | 35^{a} | 1.3 | ND | ND |
| | 8 | 90^{a} | 16 | ND | ND |
| | 8 | ND | 41 | 31-138 | 138 |

All genes were delivered as transposons. Except when noted LC and HC genes were provided on separate vectors.
^{a}LC and HC genes were coupled in the vectors.
^{b}ND, not done

The use of CHO8A cells to quickly generate selected pools of permanent transfectants expressing mAbs, as opposed to clones, is another important application. The pools produced here exhibited the substantial Qps of 20 pcd (trastuzumab) and 41 pcd (9-24). The rapid generation of CHO cell pools permanently expressing a recombinant protein has drawn more attention in recent years, in that such pools could be effectively used in preclinical development (Rajendra et al. 2017). A short time frame is especially important in the case of an emergent requirement, like the production of antibodies or vaccines targeting SARS-CoV-2. In the case of 9-24, pooled clones obtained 12 days after transfection had a Qp high enough to produce antibody for preclinical study and at the same time to go on to isolate high producers of single clones from the pool. Hence, CHO8A cells may represent a valuable alternative to the HEK293 cells commonly used for this purpose.

Two selection systems extensively used in industry to produce high amounts of therapeutic antibodies are DHFR- and GS-based. Gene amplified cells can be selected by resistance to their enzyme inhibitors methotrexate (MTX) and methionine sulfoximine (MSX) respectively and are then usually screened for high producing clones. This process can often be laborious, needing multiple rounds of incremental increases in resistance (Cacciatore et al. 2010). In addition, withdrawal of the inhibitors sometimes results in a reduction in transgene copy number and a diminishment in productivity (Wurm & Wurm, 2017). Furthermore, during downstream purification, removal of the toxic inhibitors requires resources and time, which is also the case when antibiotic selections are used. CHO8A cells offer an alternative selection system that involves neither toxic drugs nor antibiotics. The selective pressure deriving from an absence of required nutrients enables the transfectants to stably produce a recombinant protein for at least 3 months of continuous culture in standard culture media.

In the course of these experiments we found that the delivery of LC and HC genes on separate vectors yielded higher productivity than their coupled presence in a single vector. This despite the 2-fold higher delivered molarity of these chains when coupled. We speculate that this effect may be related to the more efficient assembly of the LC and HC in high producing transfectants. It is known that the ratio of LC to HC is an important variable in the assembly of functional antibodies and that this ratio is particular for different mAbs (Wijesuriya et al. 2018). The use of transposons to deliver these vectors will result in manifold independent integration sites, each with its own expression level. LC and HC genes on separate vectors may result in a wide clonal distribution of LC/HC copy number and/or expression ratios. This variability is then exploited by screening for the highest productivity clone(s). By comparison, the use of vectors with coupled LC and HC genes generates a fixed ratio, precluding this advantageous variability.

In summary, this CHO8A system provides 8 selective markers (rescue enzymes) for manipulation of transgenes. Application of CHO8A cells to produce a model antibody trastuzumab and an anti-SARS-CoV-2 antibody 9-24 demonstrated several beneficial properties of CHO8A in production of recombinant proteins: 1) rapid attainment of pooled cells or clones permanently expressing mAbs in sizable amounts; 2) no antibiotics or drugs are needed for selection and maintenance of selective pressure for long periods of culture; 3) flexibility in the allocation of transgenes, i.e., introduction of 1-8 transgenes at once or via multiple rounds of serial transfections. Thus CHO8A cells represent a powerful platform for flexible and rapid production of CHO clones expressing high levels of recombinant proteins.

All patents, patent applications, and publications cited herein are incorporated herein by reference in their entirety as if recited in full herein.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

### REFERENCES

1. Balasubramanian, S., Rajendra, Y., Baldi, L., Hacker, D. L., & Wurm, F. M. (2016). Comparison of three transposons for the generation of highly productive recombinant CHO cell pools and cell lines. Biotechnol Bioeng, 113(6), 1234-1243. doi: 10.1002/bit.25888
2. Balasubramanian, S., Wurm, F. M. & Hacker, D. L. Multigene expression in stable CHO cell pools generated with the piggyBac transposon system. Biotechnol Prog 32, 1308-1317 (2016).
3. Baresova, V., Krijt, M., Skopova, V., Souckova, O., Kmoch, S., & Zikanova, M. (2016). CRISPR-Cas9 induced mutations along de novo purine synthesis in HeLa cells result in accumulation of individual enzyme substrates and affect purinosome formation. Mol Genet Metab, 119(3), 270-277. doi: 10.1016/j.ymgme.2016.08.004
4. Cacciatore, J. J., Chasin, L. A. & Leonard, E. F. Gene amplification and vector engineering to achieve rapid and high-level therapeutic protein production using the Dhfr-based CHO cell selection system. Biotechnol Adv 28, 673-681 (2010).
5. Chusainow, J., Yang, Y. S., Yeo, J. H., Toh, P. C., Asvadi, P., Wong, N. S., & Yap, M. G. (2009). A study of monoclonal antibody-producing CHO cell lines: what makes a stable high producer? Biotechnol Bioeng, 102(4), 1182-1196. doi: 10.1002/bit.22158
6. Costa, A. R., Rodrigues, M. E., Henriques, M., Melton, D., Cunnah, P., Oliveira, R., & Azeredo, J. (2012). Evaluation of the OSCAR system for the production of monoclonal antibodies by CHO-K1 cells. Int J Pharm, 430(1-2), 42-46. doi: 10.1016/j.ijpharm.2012.03.028
7. Deng, Y. et al. NADPH-dependent GMP reductase isoenzyme of human (GMPR2). Expression, purification, and kinetic properties. Int J Biochem Cell Biol 34, 1035-1050 (2002).
8. Fischer, S., Handrick, R., & Otte, K. (2015). The art of CHO cell engineering: A comprehensive retrospect and future perspectives. Biotechnol Adv, 33(8), 1878-1896. doi: 10.1016/j.biotechadv.2015.10.015
9. Ho, S. C. et al. IRES-mediated Tricistronic vectors for enhancing generation of high monoclonal antibody expressing CHO cell lines. J Biotechnol 157, 130-139 (2012).
10. Hunting, D., Hordern, J. & Henderson, J. F. Quantitative analysis of purine and pyrimidine metabolism in Chinese hamster ovary cells. Can J Biochem 59, 838-847 (1981).
11. Izsvak Z, Chuah M K, Vandendriessche T, Ivics Z. Efficient stable gene transfer into human cells by the Sleeping Beauty transposon vectors. Methods. 2009; 49(3):287-97. doi:10.1016/j.ymeth.2009.07.001.
12. Kaas, C. S., Kristensen, C., Betenbaugh, M. J. & Andersen, M. R. Sequencing the CHO DXB11 genome reveals regional variations in genomic stability and haploidy. BMC Genomics 16, 160 (2015).
13. Kao, F. T., & Puck, T. T. (1968). Genetics of somatic mammalian cells, VII. Induction and isolation of nutritional mutants in Chinese hamster cells. Proc Natl Acad Sci USA, 60(4), 1275-1281.
14. Kingston, R. E., Kaufman, R. J., Bebbington, C. R. & Rolfe, M. R. Amplification using CHO cell expression vectors. Curr Protoc Mol Biol Chapter 16, Unit 16 23 (2002).
15. Ko, N., Nishihama, R., & Pringle, J. R. (2008). Control of 5-FOA and 5-FU resistance by Saccharomyces cerevisiae YJL055W. Yeast, 25(2), 155-160. doi: 10.1002/yea.1554
16. Kolacsek, O. et al. Reliable transgene-independent method for determining Sleeping Beauty transposon copy numbers. Mob DNA 2, 5 (2011).
17. Krooth, R. S., Hsiao, W. L., & Potvin, B. W. (1979). Resistance to 5-fluoroorotic acid and pyrimidine auxotrophy: a new bidirectional selective system for mammalian cells. Somatic Cell Genet, 5(5), 551-569.
18. Kuo, C. C., Chiang, A. W., Shamie, I., Samoudi, M., Gutierrez, J. M., & Lewis, N. E. (2018). The emerging role of systems biology for engineering protein production in CHO cells. Curr Opin Biotechnol, 51, 64-69. doi: 10.1016/j.copbio.2017.11.015
19. Lalonde, M. E., & Durocher, Y. (2017). Therapeutic glycoprotein production in mammalian cells. J Biotechnol, 251, 128-140. doi: 10.1016/j.jbiotec.2017.04.028
20. Le Fourn, V., Girod, P. A., Buceta, M., Regamey, A., & Mermod, N. (2014). CHO cell engineering to prevent polypeptide aggregation and improve therapeutic protein secretion. Metab Eng. 2014 January; 21:91-102. doi: 10.1016/j.ymben.2012.12.003.
21. Moremen. K. W., Ramiah, A., Stuart, M., Steel, J., Meng, L., Forouhar, F., Moniz, H. A., Gahlay, G., Gao, Z., Chapla, D., Wang, S., Yang, J. Y., Prabhakar, P. K., Johnson, R., Rosa, M. D., Geisler, C., Nairn, A. V., Seetharaman, J., Wu, S. C., Tong, L., Gilbert, H. J., LaBaer, J., & Jarvis, D. L. (2018). Expression system for structural and functional studies of human glycosylation enzymes. Nat Chem Biol. 2018 February; 14(2):156-162. doi: 10.1038/nchembio.2539.
22. Moritz, B., Woltering, L., Becker, P. B., & Gopfert, U. (2016). High levels of histone H3 acetylation at the CMV promoter are predictive of stable expression in Chinese hamster ovary cells. Biotechnol Prog, 32(3), 776-786. doi: 10.1002/btpr.2271
23. Naito, Y., Hino, K., Bono, H. & Ui-Tei, K. CRISPRdirect: software for designing CRISPR/Cas guide RNA with reduced off-target sites. Bioinformatics 31, 1120-1123 (2015).
24. Noh, S. M., Shin, S. & Lee, G. M. Comprehensive characterization of glutamine synthetase-mediated selection for the establishment of recombinant CHO cells producing monoclonal antibodies. Sci Rep 8, 5361 (2018).
25. Puck, T. T., & Kao, F. T. (1982). Somatic cell genetics and its application to medicine. Annu Rev Genet, 16, 225-271. doi: 10.1146/annurev.ge.16.120182.001301
26. Querques, I. et al. A highly soluble Sleeping Beauty transposase improves control of gene insertion. Nat Biotechnol 37, 1502-1512 (2019).
27. Rajendra, Y. et al. Bioreactor scale up and protein product quality characterization of piggyBac transposon derived CHO pools. Biotechnol Prog 33, 534-540 (2017).
28. Rebora, K., Laloo, B., & Daignan-Fornier, B. (2005). Revisiting purine-histidine cross-pathway regulation in Saccharomyces cerevisiae: a central role for a small molecule. Genetics, 170(1), 61-70. doi: 10.1534/genetics.104.039396
29. Rees, W. D. & Hay, S. M. The biosynthesis of threonine by mammalian cells: expression of a complete bacterial biosynthetic pathway in an animal cell. Biochem J 309 (Pt 3), 999-1007 (1995).
30. Rita Costa, A., Elisa Rodrigues, M., Henriques, M., Azeredo, J., & Oliveira, R. (2010). Guidelines to cell engineering for monoclonal antibody production. Eur J Pharm Biopharm, 74(2), 127-138. doi: 10.1016/j.ejpb.2009.10.002
31. Runcie, K., Budman, D. R., John, V., & Seetharamu, N. (2018). Bi-specific and tri-specific antibodies—the next big thing in solid tumor therapeutics. Mol Med, 24(1), 50. doi: 10.1186/s10020-018-0051-4
32. Sergeeva, D., Lee, G. M., Nielsen, L. K. & Gray, L. M. Multicopy Targeted Integration for Accelerated Development of High-Producing Chinese Hamster Ovary Cells. ACS Synth Biol 9, 2546-2561 (2020).
33. Shatz, W., Ng, D., Dutina, G., Wong, A. W., Dunshee, D. R., Sonoda, J., Shen, A., & Scheer, J. M. (2016). An efficient route to bispecific antibody production using single-reactor mammalian co-culture. MAbs. 2016 November/December; 8(8):1487-1497.
34. Trolle, J. et al. Resurrecting essential amino acid biosynthesis in a mammalian cell. bioRxiv, 2021.2008.2003.454854 (2021).
35. Wijesuriya, S. D. et al. Antibody engineering to improve manufacturability. Protein Expr Purif 149, 75-83 (2018).
36. Wurm, F. M. & Wurm, M. J. Cloning of CHO Cells, Productivity and Genetic Stability—A Discussion. Processes 5, 20 (2017).
37. Zhang, Q., Jiang, B., Du, Z. & Chasin, L. A. A doubly auxotrophic CHO-K1 cell line for the production of recombinant monoclonal antibodies. Biotechnol Bioeng 117, 2401-2409 (2020).

What is claimed is:

1. A multiply auxotrophic cell line that is deficient in (i) at least one gene encoding an enzyme in the de novo pathway for pyrimidine nucleotide synthesis and (ii) at least one gene encoding an enzyme in the de novo pathway for purine nucleotide synthesis.

2. The multiply auxotrophic cell line according to claim 1, wherein the cell line is deficient in at least two genes in the de novo pathway for pyrimidine nucleotide synthesis and/or in the de novo pathway for purine nucleotide synthesis.

3. The multiply auxotrophic cell line according to claim 1, wherein the cell line is deficient in two to thirteen genes in the de novo pathway for pyrimidine nucleotide synthesis and/or in the de novo pathway for purine nucleotide synthesis.

4. The multiply auxotrophic cell line according to claim 1, wherein the cell line is deficient in 2, 3, 4, 5, 6, 7, 8, 9, 10, 11, 12 or 13 genes in the de novo pathway for pyrimidine nucleotide synthesis and/or in the de novo pathway for purine nucleotide synthesis.

5. The multiply auxotrophic cell line according to claim 1, wherein the enzyme in the de novo pathway for pyrimidine nucleotide synthesis is uridine monophosphate synthetase (UMPS) and the enzyme in the de novo pathway for purine nucleotide synthesis is 5-aminoimidazole-4-carboxamide ribonucleotide formyltransferase/IMP cyclohydrolase (ATIC).

6. The multiply auxotrophic cell line according to claim 1, wherein the enzymes in the de novo pathway for pyrimidine nucleotide synthesis are selected from uridine monophosphate synthetase (UMPS), dihydroorotate dehydrogenase (DHODH), CTP synthase 1 and 2 (CTPS1/2) and thymidylate synthetase (TYMS), and the enzyme in the de novo pathway for purine nucleotide synthesis is 5-aminoimidazole-4-carboxamide ribonucleotide formyltransferase/IMP cyclohydrolase (ATIC).

7. The multiply auxotrophic cell line according to claim 1, wherein the enzymes in the de novo pathway for pyrimidine nucleotide synthesis are selected from uridine monophosphate synthetase (UMPS), dihydroorotate dehydrogenase (DHODH), CTP synthase 1 and 2 (CTPS1/2) and thymidylate synthetase (TYMS), and the enzymes in the de novo pathway for purine nucleotide synthesis are selected from 5-aminoimidazole-4-carboxamide ribonucleotide formyltransferase/IMP cyclohydrolase (ATIC), phosphoribosylaminoimidazole carboxylase (PAICS) and guanosine monophosphate synthetase (GMPS).

8. The multiply auxotrophic cell line according to claim 1, wherein the enzymes in the de novo pathway for pyrimidine nucleotide synthesis are selected from uridine monophosphate synthetase (UMPS), dihydroorotate dehydrogenase (DHODH), CTP synthase 1 and 2 (CTPS1/2) and thymidylate synthetase (TYMS), and the enzymes in the de novo pathway for purine nucleotide synthesis are selected from 5-aminoimidazole-4-carboxamide ribonucleotide formyltransferase/IMP cyclohydrolase (ATIC), phosphoribosylaminoimidazole carboxylase (PAICS), inosine-5′-monophosphate dehydrogenase 1 and 2 (IMPDH1/2), and guanosine monophosphate synthetase (GMPS).

9. The multiply auxotrophic cell line according to claim 1, wherein the enzymes in the de novo pathway for pyrimidine nucleotide synthesis are selected from uridine monophosphate synthetase (UMPS), dihydroorotate dehydrogenase (DHODH), CTP synthase 1 and 2 (CTPS1/2) and thymidylate synthetase (TYMS), and the enzymes in the de novo pathway for purine nucleotide synthesis are selected from 5-aminoimidazole-4-carboxamide ribonucleotide formyltransferase/IMP cyclohydrolase (ATIC), phosphoribosylaminoimidazole carboxylase (PAICS), adenylosuccinate lyase (ADSL), inosine-5′-monophosphate dehydrogenase 1 and (IMPDH1/2), guanosine monophosphate synthetase (GMPS) and adenylosuccinate synthase and adenylosuccinate synthase like 1 (ADSS/ADSSL1).

10. The multiply auxotrophic cell line according to claim 1, wherein the enzymes in the de novo pathway for pyrimidine nucleotide synthesis are selected from uridine monophosphate synthetase (UMPS), dihydroorotate dehydrogenase (DHODH), CTP synthase 1 and 2 (CTPS1/2) and thymidylate synthetase (TYMS), and the enzymes in the de novo pathway for purine nucleotide synthesis are selected from 5-aminoimidazole-4-carboxamide ribonucleotide formyltransferase/IMP cyclohydrolase (ATIC), phosphoribosylaminoimidazole carboxylase (PAICS), adenylosuccinate lyase (ADSL), inosine-5′-monophosphate dehydrogenase 1 and 2 (IMPDH1/2), guanosine monophosphate synthetase (GMPS), adenylosuccinate synthase and adenylosuccinate synthase like 1 (ADSS/ADSSL1), phosphoribosyl pyrophosphate am idotransferase (PPAT), phosphoribosylglycinam ide formyltransferase (GART) and phosphoribosylformylglycinamidine synthase (PFAS).

11. The multiply auxotrophic cell line of claim 1, wherein the cell line is selected from the group consisting of HEK293, HEK293T, BHK21, CHO, CHO/dhfr−, CHO-K1, NS0, Sp2/0-Ag14, and Sp2/0-Ag14-TurboDoma.

12. The multiply auxotrophic cell line of claim 1, wherein the cell line is a CHO cell line.

13. The multiply auxotrophic cell line of claim 1, wherein the cell line is a CHO-K1 cell line.

14. A doubly auxotrophic cell line that is deficient in the gene encoding uridine monophosphate synthetase (UMPS) and the gene encoding 5-aminoimidazole-4-carboxamide ribonucleotide formyltransferase/IMP cyclohydrolase (ATIC).

15. The doubly auxotrophic cell line of claim 14, wherein the cell line is selected from the group consisting of HEK293, HEK293T, BHK21, CHO, CHO/dhfr−, CHO-K1, NS0, Sp2/0-Ag14, and Sp2/0-Ag14-TurboDoma.

16. The doubly auxotrophic cell line of claim 14, wherein the cell line is a CHO cell line.

17. The doubly auxotrophic cell line of claim 14, wherein the cell line is a CHO-K1 cell line.

18. A method for preparing a doubly auxotrophic cell line according to claim 14, comprising the steps of:
(a) knocking out a UMPS gene from the genome of the cell line;
(b) growing cells from step (a) in a medium containing 5-fluoroorotic acid (5-FOA) and uridine;
(c) selecting cells that survive in step (b) and further knocking out an ATIC gene from the genome of the surviving cells;
(d) growing clones of cells from step (c) in duplicate in both (i) a medium containing uridine but no hypoxanthine and (ii) a complete medium; and
(e) if the cells do not survive in (d-i), collecting their counterparts in (d-ii) as the doubly auxotrophic cells.

19. The method of claim 18, wherein the ATIC and UMPS genes are knocked out by CRISPR-Cas9 vectors.

20. A method for selecting a cell expressing a protein of interest, comprising the steps of:
(a) obtaining a doubly auxotrophic cell line according to claim 14;
(b) constructing a first vector carrying a UMPS open reading frame (ORF) and a first coding sequence of the protein of interest;
(c) constructing a second vector carrying an ATIC open reading frame (ORF) and a second coding sequence of the protein of interest;
(d) transfecting the doubly auxotrophic cells with the first and second vectors;
(e) incubating the transfected cells from step (d) in a medium that lacks sources of purines and pyrimidines; and
(f) selecting a cell that survives in step (e) as the cell expressing the protein of interest.

21. The method of claim 20, wherein the cell line is selected from the group consisting of HEK293, HEK293T, BHK21, CHO, CHO/dhfr−, CHO-K1, NS0, Sp2/0-Ag14, and Sp2/0-Ag14-TurboDoma.

22. The method of claim 20, wherein the cell line is a CHO cell line.

23. The method of claim 20, wherein the cell line is a CHO-K1 cell line.

24. The method of claim 20, wherein the first coding sequence is the same as the second coding sequence.

25. The method of claim 20, wherein the first coding sequence is different from the second coding sequence.

26. The method of claim 20, wherein the protein of interest is a recombinant protein selected from the group consisting of a decoy receptor, an enzyme used in an enzyme replacement therapy (ERT), a metabolic modulator, a trifunctional bispecific antibody, and a monoclonal antibody (mAb).

27. The method of claim 20, wherein the protein of interest is a monoclonal antibody (mAb).

28. A method for producing a protein of interest, comprising:
(a) obtaining a doubly auxotrophic cell line according to claim 14;
(b) constructing a first vector carrying a UMPS open reading frame (ORF) and a first coding sequence of the protein of interest;
(c) constructing a second vector carrying an ATIC open reading frame (ORF) and a second coding sequence of the protein of interest;
(d) transfecting the doubly auxotrophic cells with the first and second vectors;
(e) incubating the transfected cells from step (d) in a medium that lacks sources of purines and pyrimidines;
(f) selecting a cell that survives in step (e) as the cell expressing the protein of interest; and
(g) producing the protein of interest by culturing the cell selected in step (f).

29. The method of claim 28, wherein the cell line is selected from the group consisting of HEK293, HEK293T, BHK21, CHO, CHO/dhfr−, CHO-K1, NS0, Sp2/0-Ag14, and Sp2/0-Ag14-TurboDoma.

30. The method of claim 28, wherein the cell line is a CHO cell line.

31. The method of claim 28, wherein the cell line is a CHO-K1 cell line.

32. The system of claim 28, wherein the first coding sequence is the same as the second coding sequence.

33. The system of claim 28, wherein the first coding sequence is different from the second coding sequence.

34. The method of claim 28, wherein the protein of interest is a recombinant protein selected from the group consisting of a decoy receptor, an enzyme used in an enzyme replacement therapy (ERT), a metabolic modulator, a trifunctional bispecific antibody, and a monoclonal antibody (mAb).

35. The method of claim 28, wherein the protein of interest is a monoclonal antibody (mAb).

36. The method of claim 35, wherein the first coding sequence encodes the light chain of the monoclonal antibody and the second coding sequence encodes the heavy chain of the monoclonal antibody.

37. The method of claim 35, wherein the first coding sequence encodes the heavy chain of the monoclonal antibody and the second coding sequence encodes the light chain of the monoclonal antibody.

38. The method of claim 28, wherein the doubly auxotrophic cells in step (d) are transfected with equal ratio of the first and second vectors.

39. The method of claim 28, wherein the doubly auxotrophic cells in step (d) are transfected with unequal ratio of the first and second vectors.

40. The method of claim 28, wherein the UMPS ORF and/or the ATIC ORF are mutated.

41. The method of claim 28, wherein the first and/or second vectors further contain an epigenetic regulatory element.

42. The method of claim 41, wherein the epigenetic regulatory element is selected from the group consisting of MARs, UCOE, STARs, and combinations thereof.

43. The method of claim 41, wherein the epigenetic regulatory element is selected from the group consisting of Human MAR 1-68, Human MAR X-29, Murine MAR S4, Chicken Lysozyme MAR, Human MAR 1-68 Core+flanking region, 4X Core MAR X29, Chicken beta-globin HS4 Insulator, UCOE from the HNRPA2B1-CBX3 locus, STAR Element 7, STAR Element 40, and combinations thereof.

44. The method of claim 28, wherein the protein of interest is a bispecific monoclonal antibody (BsMAb).

45. The method of 44, wherein:
i) the first vector is a tricistronic vector and the first coding sequence encodes a heavy chain and a light chain from a first monoclonal antibody;
ii) the second vector is a tricistronic vector and the second coding sequence encodes a heavy chain and a light chain from a second monoclonal antibody; and
iii) the first monoclonal antibody is different from the second monoclonal antibody.

46. A kit for selecting a cell expressing a protein of interest, comprising:
i) a doubly auxotrophic cell line according to claim 14;
ii) a first vector carrying a UMPS open reading frame (ORF) and a first coding sequence of the protein of interest;
iii) a second vector carrying an ATIC open reading frame (ORF) and a second coding sequence of the protein of interest;
iv) a medium that lacks sources of purines and pyrimidines; and
v) instructions of use.

47. The kit of of claim 46, wherein the cell line is selected from the group consisting of HEK293, HEK293T, BHK21, CHO, CHO/dhfr−, CHO-K1, NS0, Sp2/0-Ag14, and Sp2/0-Ag14-TurboDoma.

48. The kit of claim 46, wherein the cell line is a CHO cell line.

49. The kit of claim 46, wherein the cell line is a CHO-K1 cell line.

50. The kit of claim 46, wherein the first coding sequence is the same as the second coding sequence.

51. The kit of claim 46, wherein the first coding sequence is different from the second coding sequence.

52. The kit of claim 46, wherein the protein of interest is a monoclonal antibody (mAb).

53. A recombinant protein made by the process of claim 28.

54. A monoclonal antibody made by the process of claim 28.

55. A bispecific antibody made by the process of claim 45.

56. A multiply auxotrophic cell line that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), and the gene encoding 5-aminoimidazole-4-carboxamide ribonucleotide formyltransferase/IMP cyclohydrolase (ATIC).

57. A multiply auxotrophic cell line that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), the gene encoding 5-aminoimidazole-4-carboxamide ribonucleotide formyltransferase/IMP cyclohydrolase (ATIC), the gene encoding phosphoribosylaminoimidazole carboxylase (PAICS), and the gene encoding guanosine monophosphate synthetase (GMPS).

58. A multiply auxotrophic cell line that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), the gene encoding 5-aminoimidazole-4-carboxamide ribonucleotide formyltransferase/IMP cyclohydrolase (ATIC), the gene encoding phosphoribosylaminoimidazole carboxylase (PAICS), the genes encoding inosine-5′-monophosphate dehydrogenase 1 and 2 (IMPDH1/2), and the gene encoding guanosine monophosphate synthetase (GMPS).

59. A multiply auxotrophic cell line that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), the gene encoding 5-aminoimidazole-4-carboxamide ribonucleotide formyltransferase/IMP cyclohydrolase (ATIC), the gene encoding phosphoribosylaminoimidazole carboxylase (PAICS), the gene encoding adenylosuccinate lyase (ADSL), the genes encoding inosine-5′-monophosphate dehydrogenase 1 and 2 (IMPDH1/2), the gene encoding guanosine monophosphate synthetase (GMPS), and the genes encoding adenylosuccinate synthase and adenylosuccinate synthase like 1 (ADSS/ADSSL1).

60. A method for modulating recombinant monoclonal antibody production, comprising:
(a) obtaining a multiply auxotrophic cell line according to claim 56;
(b) constructing a vector carrying
i) the open reading frame (ORF) of a required enzyme; and
ii) a coding sequence of the heavy chain of the recombinant monoclonal antibody; or
a coding sequence of the light chain of the recombinant monoclonal antibody;
(c) constructing another vector according to step (b) with a different required enzyme;
(d) repeating step (c) until each of the required enzymes is carried by one vector, and at least one of the vectors carries the coding sequence of the heavy chain or light chain of the recombinant monoclonal antibody or each of the vectors carries the coding sequence of the heavy chain or light chain of the recombinant monoclonal antibody;
(e) transfecting the multiply auxotrophic cells with all the vectors;
(f) incubating the transfected cells from step (e) in a medium that lacks sources of purines and pyrimidines;
(g) selecting a cell that survives in step (f) as the cell expressing the recombinant monoclonal antibody; and
(h) producing the recombinant monoclonal antibody by culturing the cell selected in step (g).

61. The method of claim 60, wherein the ratio of vectors carrying the coding sequence of the heavy chain of the recombinant monoclonal antibody and vectors carrying the coding sequence of the light chain of the recombinant monoclonal antibody is designed to optimize the recombinant monoclonal antibody production.

62. A method for producing a multi-subunit protein of interest, comprising:
(a) obtaining a multiply auxotrophic cell line according to claim 56;
(b) constructing a vector carrying
i) the open reading frame (ORF) of a required enzyme; and
ii) the coding sequence of a subunit of the protein of interest;
(c) constructing another vector according to step (b) with a different required enzyme and the coding sequence of a different subunit of the protein of interest;
(d) repeating step (c) until each subunit of the protein of interest is carried by at least one vector carrying a different required enzyme;
(e) transfecting the multiply auxotrophic cells with all the vectors;
(f) incubating the transfected cells from step (e) in a medium that lacks sources of purines and pyrimidines;
(g) selecting a cell that survives in step (f) as the cell expressing the multi-subunit protein of interest; and
(h) producing the multi-subunit protein of interest by culturing the cell selected in step (g).

63. The method of claim 62, wherein the multi-subunit protein of interest is a recombinant protein selected from the group consisting of a decoy receptor, an enzyme used in an enzyme replacement therapy (ERT), a metabolic modulator, a trifunctional bispecific antibody, and a monoclonal antibody (mAb).

64. The method of claim 62, wherein the multi-subunit protein of interest can be a combination of polypeptides of the signal recognition particle (SRP) subunits, ATP synthase, cleavage and polyadenylation specificity factor (CPSF), a monoclonal antibody, a trifunctional bispecific antibody, and combinations thereof.

65. The method of claim 62, wherein the multi-subunit protein of interest is a trifunctional bispecific antibody.

66. A method for optimizing the activity of a protein of interest, comprising:
(a) obtaining a multiply auxotrophic cell line that expresses the protein of interest according to claim 56;
(b) constructing a vector carrying
i) the open reading frame (ORF) of a required enzyme; and
ii) the coding sequence of an enzyme that can modulate the activity of the protein of interest;
(c) constructing another vector according to step (b) with a different required enzyme and the coding sequence of a different enzyme that can modulate the activity of the protein of interest;
(d) repeating step (c) as necessary until each enzyme that can modulate the activity of the protein of interest is carried by at least one vector carrying a different required enzyme;
(e) transfecting the multiply auxotrophic cells with all the vectors;
(f) incubating the transfected cells from step (e) in a medium that lacks sources of purines and pyrimidines;
(g) selecting a cell that survives in step (f) as the cell expressing the protein of interest having desired activity; and
(h) producing the protein of interest having desired activity by culturing the cell selected in step (g).

67. The method of claim 66, wherein the enzyme that can modulate the activity of the protein of interest is involved in a post-translational modification (PTM) of the protein of interest.

68. The method of claim 67, wherein the post-translational modification (PTM) is selected from the group consisting of myristoylation, palmitoylation, isoprenylation, prenylation, glypiatyon, lipoylation, phophopantetheinylation, acylation, acetylation, formylation, alkylation, methylation, amidation, arginylation, polyglutamylation, polyglycylation, butyrylation, gamma-carboxylation, glycosylation, N-linked glycosylation, O-linked glycosylation, polysialylation, malonylation, hydroxylation, iodination, ADP-ribosylation, phosphorylation, adenylylation, uridylylation, propionylation, pyroglutamate formation, S-glutathionylation, S-nitrosylation, S-sulfenylation, S-sulfinylation, S-sulfonylation, succinylation, sulfation, glycation, carbamylation, carbonylation, biotinylation, carbamylation, oxidation, pegylation, spontaneous isopeptide bond formation, ISGylation, SUMOylation, ubiquitination, Neddylation, Pupylation, citrullination, deamidation, eliminylation, disulfide bridge formation, proteolytic cleavage, isoaspartate formation, racemization, protein splicing, and combinations thereof.

69. The method of claim 66, the enzyme that can modulate the activity of the protein of interest is a glycosyltransferase or a hydrolase.

70. The method of claim 66, wherein the protein of interest is a recombinant protein selected from the group consisting of a decoy receptor, an enzyme used in an enzyme replacement therapy (ERT), a metabolic modulator, a trifunctional bispecific antibody, and a monoclonal antibody (mAb).

71. An octa-auxotrophic cell line that is deficient in the gene encoding uridine monophosphate synthetase (UMPS), the gene encoding dihydroorotate dehydrogenase (DHODH), the genes encoding CTP synthase 1 and 2 (CTPS1/2), the gene encoding thymidylate synthetase (TYMS), the gene encoding 5-aminoimidazole-4-carboxamide ribonucleotide formyltransferase/IMP cyclohydrolase (ATIC), the gene encoding phosphoribosylaminoimidazole carboxylase (PAICS), the genes encoding inosine-5′-monophosphate dehydrogenase 1 and 2 (IMPDH1/2), and the gene encoding guanosine monophosphate synthetase (GMPS).

72. The octa-auxotrophic cell line of claim 71, wherein the cell line is selected from the group consisting of HEK293, HEK293T, BHK21, CHO, CHO/dhfr−, CHO-K1, NS0, Sp2/0-Ag14, and Sp2/0-Ag14-TurboDoma.

73. The octa-auxotrophic cell line of claim 71, wherein the cell line is a CHO cell line.

74. The octa-auxotrophic cell line of claim 71, wherein the cell line is a CHO-K1 cell line.

75. A method for preparing an octa-auxotrophic cell line according to claim 71, comprising the steps of:
(a) knocking out the gene encoding UMPS and the gene encoding ATIC from the genome of a cell line to produce a doubly auxotrophic cell line;
(b) knocking out genes DHODH, TYMS, CTPS1 and CTPS2 from the genome of the doubly auxotrophic cell line obtained in step (a);
(c) growing colonies of cells from step (b) in duplicate in both (i) a selective medium containing none of thymidine, cytidine, uridine and hypoxanthine and (ii) a complete medium;
(d) if the cells do not survive in (c-i), collecting their counterparts in (c-ii) and further confirming the knock-out of DHODH, TYMS, CTPS1 and CTPS2 by DNA-sequencing;
(e) selecting cells with confirmed knock-out of DHODH, TYMS, CTPS1 and CTPS2 in step (d) and further knocking out genes GMPS and PAICS from the genome of the selected cells;
(f) growing clones of cells from step (e) in duplicate in both (i) a selective medium containing none of thymidine, cytidine, guanine, uridine and hypoxanthine and (ii) a complete medium;
(g) if the cells do not survive in (f-i), collecting their counterparts in (f-ii) and further confirming the knock-out of GMPS and PAICS by DNA-sequencing;
(h) selecting cells with confirmed knock-out of GMPS and PAICS in step (g) and further knocking out genes IMPDH1 and IMPDH2 from the genome of the selected cells;
(i) growing clones of cells from step (h) in a complete medium and confirming the knock-out of IMPDH1 and IMPDH2 by DNA-sequencing; and
(j) selecting the cells confirmed in step (i) as the octa-auxotrophic cells.

76. The method of claim 75, wherein the DHODH, TYMS, CTPS1, CTPS2, GMPS, PAICS, IMPDH1 and IMPDH2 genes are knocked out by CRISPR-Cas9 vectors.

77. A method for modulating recombinant monoclonal antibody production, comprising:
(a) constructing a vector carrying
i) the open reading frame (ORF) of a required enzyme for an octa-auxotrophic cell line; and
ii) a coding sequence of the heavy chain of the recombinant monoclonal antibody; or
a coding sequence of the light chain of the recombinant monoclonal antibody;
(b) constructing another vector according to step (a) with a different required enzyme;
(c) repeating step (b) until each of the required enzymes is carried by one vector, and at least one of the vectors carries the coding sequence of the heavy chain or light chain of the recombinant monoclonal antibody or each of the vectors carries the coding sequence of the heavy chain or light chain of the recombinant monoclonal antibody;
(d) transfecting the octa-auxotrophic cell line with all the vectors;
(e) incubating the transfected cells from step (d) in a medium that lacks sources of purines and pyrimidines;
(f) selecting a cell that survives in step (e) as the cell expressing the recombinant monoclonal antibody; and
(g) producing the recombinant monoclonal antibody by culturing the cell selected in step (f).

78. A method for modulating recombinant monoclonal antibody production, comprising:
(a) constructing a vector carrying
i) the open reading frame (ORF) of a required enzyme for an octa-auxotrophic cell line; and
ii) a coding sequence of the heavy chain of the recombinant monoclonal antibody; and
a coding sequence of the light chain of the recombinant monoclonal antibody;
(b) constructing another vector according to step (a) with a different required enzyme;
(c) repeating step (b) until each of the required enzymes is carried by one vector;
(d) transfecting the octa-auxotrophic cell line with all the vectors;
(e) incubating the transfected cells from step (e) in a medium that lacks sources of purines and pyrimidines;
(f) selecting a cell that survives in step (e) as the cell expressing the recombinant monoclonal antibody; and
(g) producing the recombinant monoclonal antibody by culturing the cell selected in step (f).

79. The method of claim 78, wherein the vector constructed in step (b) carries more copies of the coding sequence of the light chain of the recombinant monoclonal antibody than the coding sequence of the heavy chain of the recombinant monoclonal antibody.

80. The method of claim 79, wherein the ratio between the copies of the coding sequence of the light chain and the heavy chain is 4 to 1.

81. A method for protein production, comprising:
(a) constructing a vector carrying
i) the open reading frame (ORF) of a required enzyme for an octa-auxotrophic cell line; and
ii) a coding sequence of one or more proteins or protein subunits of interest;
(b) constructing another vector according to step (a) with a different required enzyme;
(c) repeating step (b) until each of the required enzymes is carried by at least one vector, and at least one of the vectors carries the coding sequence of the one or more proteins or protein subunits of interest or each of the vectors carries the coding sequence of the one or more proteins or protein subunits of interest;
(d) transfecting the octa-auxotrophic cell line with the constructed vectors;
(e) incubating the transfected cells from step (d) in a medium that lacks sources of purines and pyrimidines;
(f) selecting a cell that survives in step (e) as the cell expressing the one or more protein or protein subunits of interest; and
(g) producing the one or more protein or protein subunits by culturing the cell selected in step (f).

82. An octa-auxotrophic cell line made by the process of claim 75.

83. The method of claim 75, wherein the cell line is selected from the group consisting of HEK293, HEK293T, BHK21, CHO, CHO/dhfr−, CHO-K1, NS0, Sp2/0-Ag14, and Sp2/0-Ag14-TurboDoma.

84. The method of claim 75, wherein the cell line is a CHO cell line.

85. The method of claim 75, wherein the cell line is a CHO-K1 cell line.

86. The method of claim 28, wherein the protein of interest is effective as an antigen for vaccine production.

87. The method of claim 28, wherein the protein of interest is selected from the group consisting of the spike protein subunits and the NP protein of SARS Coy 2 virus and the gp120 envelope protein from the HIV virus, and combinations thereof.

88. The method of claim 81, wherein the one or more protein or protein subunits of interest are effective as an antigen for vaccine production.

89. The method of claim 81, wherein the one or more protein or protein subunits of interest are selected from the group consisting of the spike protein subunits and the NP protein of SARS Coy 2 virus and the gp120 envelope protein from the HIV virus, and combinations thereof.


SEQUENCE LISTING (133 sequences)

SEQ ID NO: 1 - DNA, 200 bp, Artificial Sequence
  Oligo
caaattgcaagctcaggggatccgtctccactccgtgtgcacgttgtccaaaatgctggagattctcgagcagcagaaaaaaattgatgccgagatggtggggagagtgaagaggttcattcaggagaatgttttcatagcaactaatcacaatggtgttccccctcctgagaagaaagcatgcaaagaactcagctttg

SEQ ID NO: 2 - DNA, 156 bp, Artificial Sequence
  Oligo
ggagaagacctggtgaagtggaaggcactgttcgaggaagtccctgagttactcactgaggcagagaagaaggaatgggtcgacaaattgagagacgtttctgtcagctccgatgccttctttcctttcagagataacgtggaccgagccaaaaga

SEQ ID NO: 3 - DNA, 20 bp, Artificial Sequence
  Oligo
cctgaaggtgactgatgcca

SEQ ID NO: 4 - DNA, 19 bp, Artificial Sequence
  Oligo
ttttgaggcaagtgggtgg

SEQ ID NO: 5 - DNA, 20 bp, Artificial Sequence
  Oligo
agcccaagtgatttctggca

SEQ ID NO: 6 - DNA, 20 bp, Artificial Sequence
  Oligo
tcagcctcaaaggcagatgg

SEQ ID NO: 7 - DNA, 70 bp, Artificial Sequence
  Oligo
atcttgtggaaaggacgaaacaccgctgcatgccgaacaccgacagttttagagctagaaatagcaagtt

SEQ ID NO: 8 - DNA, 36 bp, Artificial Sequence
  Oligo
tctaccgggtacccttgttgcggtgtttcgtccttt

SEQ ID NO: 9 - DNA, 45 bp, Artificial Sequence
  Oligo
caacaagggtacccggtagagttttagagctagaaatagcaagtt

SEQ ID NO: 10 - DNA, 36 bp, Artificial Sequence
  Oligo
cataccgatgtctgtgtcttcggtgtttcgtccttt

SEQ ID NO: 11 - DNA, 45 bp, Artificial Sequence
  Oligo
aagacacagacatcggtatggttttagagctagaaatagcaagtt

SEQ ID NO: 12 - DNA, 36 bp, Artificial Sequence
  Oligo
taccggatttcggaatttatcggtgtttcgtccttt

SEQ ID NO: 13 - DNA, 45 bp, Artificial Sequence
  Oligo
ataaattccgaaatccggtagttttagagctagaaatagcaagtt

SEQ ID NO: 14 - DNA, 36 bp, Artificial Sequence
  Oligo
acttgctatttctagctctacggtgtttcgtccttt

SEQ ID NO: 15 - DNA, 70 bp, Artificial Sequence
  Oligo
atcttgtggaaaggacgaaacaccgtcaaggttgtagcgcgctctgttttagagctagaaatagcaagtt

SEQ ID NO: 16 - DNA, 36 bp, Artificial Sequence
  Oligo
ctgggcagtcgaagggatgacggtgtttcgtccttt

SEQ ID NO: 17 - DNA, 45 bp, Artificial Sequence
  Oligo
tcatcccttcgactgcccaggttttagagctagaaatagcaagtt

SEQ ID NO: 18 - DNA, 36 bp, Artificial Sequence; identical to SEQ ID NO: 14
  Oligo

SEQ ID NO: 19 - DNA, 70 bp, Artificial Sequence
  Oligo
atcttgtggaaaggacgaaacaccggccaccaccagctcgatccggttttagagctagaaatagcaagtt

SEQ ID NO: 20 - DNA, 36 bp, Artificial Sequence; identical to SEQ ID NO: 14
  Oligo

SEQ ID NO: 21 - DNA, 70 bp, Artificial Sequence
  Oligo
atcttgtggaaaggacgaaacaccggccaagaacctcatagacgcgttttagagctagaaatagcaagtt

SEQ ID NO: 22 - DNA, 36 bp, Artificial Sequence; identical to SEQ ID NO: 14
  Oligo

SEQ ID NO: 23 - DNA, 43 bp, Artificial Sequence
  Oligo
tcccaggtccaactgcaggtcgagcgccgccaccatgtccgtg

SEQ ID NO: 24 - DNA, 49 bp, Artificial Sequence
  Oligo
agaggggcggaattggccgccctagatgcatcagcactcgccccggttg

SEQ ID NO: 25 - DNA, 44 bp, Artificial Sequence
  Oligo
tcccaggtccaactgcaggtcgagcgccgccaccatggaatggt

SEQ ID NO: 26 - DNA, 49 bp, Artificial Sequence
  Oligo
agaggggcggaattggccgccctagatgcatcacttgccggggctcaga

SEQ ID NO: 27 - DNA, 50 bp, Artificial Sequence
  Oligo
agccatagagcccaccgcatccccagcatgcgatgtacgggccagatata

SEQ ID NO: 28 - DNA, 50 bp, Artificial Sequence
  Oligo
cccgagtagctagttcatggcagccagcatgtcgacggtatacagacatg

SEQ ID NO: 29 - DNA, 53 bp, Artificial Sequence
  Oligo
gcggcctagctagcgcttaaggcctgttaaccggtgccgccaccatgtccgtg

SEQ ID NO: 30 - DNA, 49 bp, Artificial Sequence
  Oligo
ggagaggggcggaattggccgccctagatgtcagcactcgccccggttg

SEQ ID NO: 31 - DNA, 18 bp, Artificial Sequence
  Oligo
catctagggcggccaatt

SEQ ID NO: 32 - DNA, 53 bp, Artificial Sequence
  Oligo
aggaccattccatggttgtggccatattatcatcgtgtttttcaaaggaaaac

SEQ ID NO: 33 - DNA, 53 bp, Artificial Sequence
  Oligo
gaaaaacacgatgataatatggccacaaccatggaatggtcctgggtgttcct

SEQ ID NO: 34 - DNA, 50 bp, Artificial Sequence
  Oligo
agaggggcggaattggccgccctagatgcattcacttgccggggctcaga

SEQ ID NO: 35 - DNA, 20 bp, Artificial Sequence
  Oligo
ataaattccgaaatccggta

SEQ ID NO: 36 - DNA, 20 bp, Artificial Sequence
  Oligo
caacaagggtacccggtaga

SEQ ID NO: 37 - DNA, 20 bp, Artificial Sequence
  Oligo
aagacacagacatcggtatg

SEQ ID NO: 38 - DNA, 20 bp, Artificial Sequence
  Oligo
ctgcatgccgaacaccgaca

SEQ ID NO: 39 - DNA, 20 bp, Artificial Sequence
  Oligo
tcatcccttcgactgcccag

SEQ ID NO: 40 - DNA, 20 bp, Artificial Sequence
  Oligo
tcaaggttgtagcgcgctct

SEQ ID NO: 41 - DNA, 20 bp, Artificial Sequence
  Oligo
gccaccaccagctcgatccg

SEQ ID NO: 42 - DNA, 20 bp, Artificial Sequence
  Oligo
gccaagaacctcatagacgc

SEQ ID NO: 43 - DNA, 360 bp, Artificial Sequence
  Oligo
gttttagagctagaaatagcaagttaaaataaggctagtccgttatcaacttgaaaaagtggcaccgagtcggtgctttttttcggtgcttttttaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttagatttcttggctttatatatcttgtggaaaggacgaaacaccg

SEQ ID NO: 44 - DNA, 590 bp, Artificial Sequence
  Oligo
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatg

SEQ ID NO: 45 - DNA, 42 bp, Artificial Sequence
  Oligo
ctcgagcagcagaaaaaaattgatgccgagatggtggggaga

SEQ ID NO: 46 - DNA, 44 bp, Artificial Sequence
  Oligo
ctcgagcagcagaaaaaaattgatgccgaagatggtggggagag

SEQ ID NO: 47 - DNA, 35 bp, Artificial Sequence
  Oligo
ctcgagcagcagaaaaaaattgatggtggggagag

SEQ ID NO: 48 - DNA, 37 bp, Artificial Sequence
  Oligo
ggagaagacctggtgaagtggaaggcactgttcgagg

SEQ ID NO: 49 - DNA, 38 bp, Artificial Sequence
  Oligo
ggagaagacctggtgaagtggaaggcactgtttcgagg

SEQ ID NO: 50 - DNA, 167 bp, Artificial Sequence
  Oligo
  misc_feature (32)..(161): n is a, c, g, or t
ggagaagacctggtgaagtggaaggcactgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcgagg

SEQ ID NO: 51 - DNA, 28 bp, Artificial Sequence
  Oligo
ggagaagacctggtgaagtggaaggcac

SEQ ID NO: 52 - DNA, 35 bp, Artificial Sequence
  Oligo
tgggccataaattccgaaatccgcggtagggattg

SEQ ID NO: 53 - DNA, 35 bp, Artificial Sequence
  Oligo
tttcatccatctaaccgggtacccttgttgttaga

SEQ ID NO: 54 - DNA, 35 bp, Artificial Sequence
  Oligo
agaagaaagacacagacatcggttatgaggtatgc

SEQ ID NO: 55 - DNA, 33 bp, Artificial Sequence
  Oligo
ctggcaccttcggtgttcggcatgcaggcgcga

SEQ ID NO: 56 - DNA, 31 bp, Artificial Sequence
  Oligo
ctggcaccttgtgttcggcatgcaggcgcga

SEQ ID NO: 57 - DNA, 31 bp, Artificial Sequence
  Oligo
gtgtggtcatcccttcgactgtggtaagatc

SEQ ID NO: 58 - DNA, 35 bp, Artificial Sequence
  Oligo
atggattcaaggttgtagcgcgcttctgggaatat

SEQ ID NO: 59 - DNA, 35 bp, Artificial Sequence
  Oligo
atggattcaaggttgtagcgcgctactgggaatat

SEQ ID NO: 60 - DNA, 30 bp, Artificial Sequence
  Oligo
atgacaccacgagctggtggtggccccagc

SEQ ID NO: 61 - DNA, 117 bp, Artificial Sequence
  Oligo
  misc_feature (10)..(102): n is a, c, g, or t
ttgtgaccannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggtggtggccccagc

SEQ ID NO: 62 - DNA, 31 bp, Artificial Sequence
  Oligo
gctcaagccaagaacctcatagaggtgtgga

SEQ ID NO: 63 - DNA, 35 bp, Artificial Sequence
  Oligo
gctcaagccaagaacctcatagaacgcaggtgtgg

SEQ ID NO: 64 - DNA, 20 bp, Artificial Sequence
  Oligo
gaaaaaaattgatgccgaga

SEQ ID NO: 65 - DNA, 20 bp, Artificial Sequence
  Oligo
gaagtggaaggcactgttcg

SEQ ID NO: 66 - DNA, 20 bp, Artificial Sequence
  Oligo
ataaattccgaaatccggta

SEQ ID NO: 67 - DNA, 20 bp, Artificial Sequence
  Oligo
caacaagggtacccggtaga

SEQ ID NO: 68 - DNA, 20 bp, Artificial Sequence
  Oligo
aagacacagacatcggtatg

SEQ ID NO: 69 - DNA, 20 bp, Artificial Sequence
  Oligo
ctgcatgccgaacaccgaca

SEQ ID NO: 70 - DNA, 20 bp, Artificial Sequence
  Oligo
tcatcccttcgactgcccag

SEQ ID NO: 71 - DNA, 20 bp, Artificial Sequence
  Oligo
tcaaggttgtagcgcgctct

SEQ ID NO: 72 - DNA, 20 bp, Artificial Sequence
  Oligo
gccaccaccagctcgatccg

SEQ ID NO: 73 - DNA, 20 bp, Artificial Sequence
  Oligo
gccaagaacctcatagacgc

SEQ ID NO: 74 - DNA, 43 bp, Artificial Sequence; identical to SEQ ID NO: 23
  Oligo

SEQ ID NO: 75 - DNA, 48 bp, Artificial Sequence
  Oligo
gaggggcggaattggccgccctagatgcatcagcactcgccccggttg

SEQ ID NO: 76 - DNA, 44 bp, Artificial Sequence; identical to SEQ ID NO: 25
  Oligo

SEQ ID NO: 77 - DNA, 49 bp, Artificial Sequence; identical to SEQ ID NO: 26
  Oligo

SEQ ID NO: 78 - DNA, 50 bp, Artificial Sequence; identical to SEQ ID NO: 27
  Oligo

SEQ ID NO: 79 - DNA, 50 bp, Artificial Sequence; identical to SEQ ID NO: 28
  Oligo

SEQ ID NO: 80 - DNA, 21 bp, Artificial Sequence
  Oligo
ccttgcaggtgataaggcaaa

SEQ ID NO: 81 - DNA, 20 bp, Artificial Sequence
  Oligo
ggaaagcactcgtggatggt

SEQ ID NO: 82 - DNA, 14 bp, Artificial Sequence
  Oligo
tcgtggtggctgcg

SEQ ID NO: 83 - DNA, 22 bp, Artificial Sequence
  Oligo
ccatgtggaacctgaacaagtg

SEQ ID NO: 84 - DNA, 21 bp, Artificial Sequence
  Oligo
caagggtacccggtagatgga

SEQ ID NO: 85 - DNA, 19 bp, Artificial Sequence
  Oligo
tctgtgttcatgatgtttc

SEQ ID NO: 86 - DNA, 17 bp, Artificial Sequence
  Oligo
acggcccaggacaagga

SEQ ID NO: 87 - DNA, 20 bp, Artificial Sequence
  Oligo
ccatcaatgcccagctctct

SEQ ID NO: 88 - DNA, 16 bp, Artificial Sequence
  Oligo
acattgccagtgtggc

SEQ ID NO: 89 - DNA, 21 bp, Artificial Sequence
  Oligo
gcaaagtcatcgaccgaagag

SEQ ID NO: 90 - DNA, 20 bp, Artificial Sequence
  Oligo
tatggcgaaggctggtgttt

SEQ ID NO: 91 - DNA, 17 bp, Artificial Sequence
  Oligo
cgggagctctttgtgca

SEQ ID NO: 92 - DNA, 21 bp, Artificial Sequence
  Oligo
ggcttcatccaccacaactgt

SEQ ID NO: 93 - DNA, 29 bp, Artificial Sequence
  Oligo
tgaatccctgttcatatttctttactttc

SEQ ID NO: 94 - DNA, 15 bp, Artificial Sequence
  Oligo
ccaggccaatgaagt

SEQ ID NO: 95 - DNA, 22 bp, Artificial Sequence
  Oligo
aaggcgatggcatacctactgt

SEQ ID NO: 96 - DNA, 19 bp, Artificial Sequence
  Oligo
ggcccaaaccattgcttct

SEQ ID NO: 97 - DNA, 16 bp, Artificial Sequence
  Oligo
ttgtcgcagtggctgg

SEQ ID NO: 98 - DNA, 16 bp, Artificial Sequence
  Oligo
cggtgttcggcatgca

SEQ ID NO: 99 - DNA, 23 bp, Artificial Sequence
  Oligo
ggttgtgagcagaggaaattcat

SEQ ID NO: 100 - DNA, 17 bp, Artificial Sequence
  Oligo
cgcgatacagcctgaga

SEQ ID NO: 101 - DNA, 20 bp, Artificial Sequence
  Oligo
ctggtcaccgagctgtacga

SEQ ID NO: 102 - DNA, 18 bp, Artificial Sequence
  Oligo
ggtgagcccgctcttcag

SEQ ID NO: 103 - DNA, 15 bp, Artificial Sequence
  Oligo
caggctttcaagttc

SEQ ID NO: 104 - DNA, 22 bp, Artificial Sequence
  Oligo
caccaacggctacaccagatac

SEQ ID NO: 105 - DNA, 18 bp, Artificial Sequence
  Oligo
ggaggtgtcggcggagat

SEQ ID NO: 106 - DNA, 13 bp, Artificial Sequence
  Oligo
agggcagattcac

SEQ ID NO: 107 - DNA, 17 bp, Artificial Sequence
  Oligo
cctggcaaggcccctaa

SEQ ID NO: 108 - DNA, 21 bp, Artificial Sequence
  Oligo
cacgccagagtacaggaagga

SEQ ID NO: 109 - DNA, 17 bp, Artificial Sequence
  Oligo
ctgctgatctactccgc

SEQ ID NO: 110 - DNA, 29 bp, Artificial Sequence
  Oligo
ctcgtttttcaactactccacaaatttct

SEQ ID NO: 111 - DNA, 26 bp, Artificial Sequence
  Oligo
gtgtcatgcacaaagtagatgtccta

SEQ ID NO: 112 - DNA, 35 bp, Artificial Sequence
  Oligo
gctgaaatgaatcattctctctactattattctga

SEQ ID NO: 113 - DNA, 26 bp, Artificial Sequence
  Oligo
aattccctgtcttaggtcagttagga

SEQ ID NO: 114 - DNA, 25 bp, Artificial Sequence
  Oligo
tggataaaattgcttcattccctga

SEQ ID NO: 115 - DNA, 25 bp, Artificial Sequence
  Oligo
aagatattgactctaacggaggaac

SEQ ID NO: 116 - DNA, 100 bp, Artificial Sequence
  Oligo
gatcattgaggatgtcgtcaccagtggggccagtgttttggaaactgttgaagttcttcagaaggagggcctgaaggtgactgatgccatagtgctgtta

SEQ ID NO: 117 - DNA, 100 bp, Artificial Sequence
  Oligo
ggcgccacttggctttgaaggcatttactcatactgcacaatatgatgaagcgatttcagattacttcagaaagcagtacagtaaggggatttcccagat

SEQ ID NO: 118 - DNA, 100 bp, Artificial Sequence
  Oligo
catccttgggggaggaggacttctcttcacctcttacctgacagccacgggcgatgaccatttctatgctgaatatctgatgccggccctgcagaggctg

SEQ ID NO: 119 - DNA, 100 bp, Artificial Sequence
  Oligo
agtggtagaattctcaagaaatgtgctgggatggcaagatgccaattctacagaatttgaccctaagactagtcatcctgtggtcatagacatgccagaa

SEQ ID NO: 120 - DNA, 100 bp, Artificial Sequence
  Oligo
tgtcggtgttcggcatgcaggcgcgatacagcctgagagatgaatttcctctgctcacaaccaaaagagtgttctggaagggagttttggaggagttgct

SEQ ID NO: 121 - DNA, 100 bp, Artificial Sequence
  Oligo
taaagcagagtatgaaggcgatggcatacctactgtatttgtcgcagtggctggcagaagcaatggtttgggcccagtgatgtctggtaatactgcatat

SEQ ID NO: 122 - DNA, 100 bp, Artificial Sequence
  Oligo
cgaaccttcattactagtgacttcatgactggtatagctgcaacacctggcaatgagatacctgtagaggtggtactaaagatggtcactgagataaaga

SEQ ID NO: 123 - DNA, 100 bp, Artificial Sequence
  Oligo
cccaagcaacagcagtgtacaaggtttctgagtatgctcggcgctttggtgttcctgttattgctgatggaggaatccaaaatgtgggtcatattgccaa

SEQ ID NO: 124 - DNA, 100 bp, Artificial Sequence
  Oligo
cagctgaagtccggcacagcttctgtcgtgtgcctgctgaacaacttctaccctcgggaagccaaggtgcagtggaaggtggacaatgccctgcagtccg

SEQ ID NO: 125 - DNA, 100 bp, Artificial Sequence
  Oligo
aagaccaagcctagagaggaacagtacaactccacctacagagtggtgtccgtgctgaccgtgctgcaccaggattggctgaacggcaaagagtacaagt

SEQ ID NO: 126 - DNA, 100 bp, Artificial Sequence
  Oligo
aggaagcaactagcaaaaagaactgtcataaaaaaaccagaccaagattaaaaaatgtggatcggagtagcgctcagcatttggaagtcactgttggaga

SEQ ID NO: 127 - DNA, 100 bp, Artificial Sequence
  Oligo
agttgaggatattgatagaagactcagaccagaacttaaagtacctggggcttctggccatgtctaaaatcctgaagacacaccccaagtccgtgcagtc

SEQ ID NO: 128 - DNA, 100 bp, Artificial Sequence
  Oligo
gctgcattatgtaaaggaccgattcctccgtcagctggatttcaacagctccaaagatgtagctgtgatgcagttgaggagtggttccaagtttccagtg

SEQ ID NO: 129 - DNA, 100 bp, Artificial Sequence
  Oligo
aacagatgaacgctaccggcatttaggattctctgttgatgaccttggctgctgtaaagtgatccgtcatagtctctggggtactcatgtgtttgtagga

SEQ ID NO: 130 - DNA, 100 bp, Artificial Sequence
  Oligo
tgtccgttgttggcacttaggaagagcgaaattgctcaagtggaagcctgcatttctcatgccaaagggttaagcctcttggatggcttgcaatgatagg

SEQ ID NO: 131 - DNA, 100 bp, Artificial Sequence
  Oligo
atgcccgttctatctacgttggcaatgtggactatggtgcaacagcagaagagctggaagctcactttcatggttgtggttcagtcaaccgtgtcactat

SEQ ID NO: 132 - DNA, 100 bp, Artificial Sequence
  Oligo
gtggacacccacacctcccctcagaacttcaaaagggcaacatctttttttccttcacttattgctgaaaccaagagcacaactcccattcagagaagga

SEQ ID NO: 133 - DNA, 100 bp, Artificial Sequence
  Oligo
ggttgcctatgggatagatagtctcttcagcttctatcacaacatgcccaagaatgccctttccagcggcctcatcatttcatccggctaccagtgtaca